(19) United States
(12) Patent Application Publication
(10) Pub. No.: US 2025/0034503 A1
(43) Pub. Date: Jan. 30, 2025

(54) CELL CULTURE CONSTRUCT

(71) Applicant: CELLULAR AGRICULTURE LTD, Llanelli Carmarthenshire (GB)

(72) Inventors: Moein Mir Fakhar, Llanelli Carmarthenshire (GB); Marianne Jane Ellis, Llanelli Carmarthenshire (GB)

(21) Appl. No.: 18/722,306

(22) PCT Filed: Dec. 21, 2022
(86) PCT No.: PCT/GB2022/053352

§ 371 Date: Jun. 20, 2024

(30) Foreign Application Priority Data
Dec. 22, 2021 (GB) 2118858.6

(51) Int. Cl. C12M 1/00; C12M 1/12

(52) U.S. Cl. CPC C12M 29/10; C12M 23/26; C12M 25/10; C12M 29/04

(57) ABSTRACT

The present invention relates to a construct for perfusion bioreactor cell culture. The construct comprises at least one flexible polymer sheet for rolling or folding in use, and a plurality of channels for transport of cell culture medium to cells and/or hosting cells in use. The present invention further relates to methods of forming a construct for perfusion bioreactor cell culture, a system for perfusion bioreactor cell culture, a method of culturing cells, and a comestible product obtainable by the system or method.

### FIELD OF INVENTION

The invention relates to constructs for cell culture and methods of using such cell constructs to culture cells. In particular, the invention relates to constructs for culturing cells for comestible products such as meat analogues. The invention also relates to a system including the constructs for culturing cells, methods of culturing cells using such systems and products produced by said methods.

### BACKGROUND

There is growing interest in cultured meat as a protein alternative. Lab-scale production of cultured meat in its simplest form of muscle cells or co-cultures of muscle and fat cells has been achieved, however scaling-up the process to make a viable economic product is still challenging.

Challenges to be overcome include ethical sourcing of raw materials, reducing cost of cell culture media, increasing protein yield for the muscle cell culture, and within the bioprocess itself improved energy efficiency and resource utilization and waste valorisation.

For increased process efficiencies and reduced environmental impact, bioreactors with higher cell densities allow a smaller culture volume thus reducing space requirements, labour requirements to set up and harvest the cells, and the amount of raw materials to manufacture them. Operating costs will be also lower as smaller bioreactors requiring less power and utilities. Additionally, such a bioreactor, using constructs that mimic muscle, could produce full-cut cultured meat i.e. replicating the exact structure of skeletal muscle.

Native skeletal muscle anatomy, consists of several arrays of uniaxial, striated myofibres in conjunction with fat cells, fibroblasts, capillaries and veins. A capillary is connected to most of the myofibres in a fascicle to provide blood perfusion, to provide the muscle cells with adequate oxygen and nutrients and also take away cell metabolism waste. This structure is well-replicated in a channelled perfusion bioreactor (PFB) where the inlet media carries oxygen and nutrients, goes through the channels and nourishes the cells and the perfused flow carries out the wastes at an outlet.

Although the concept of a channelled PFB seems to satisfy the targeted milestones to have higher cell density and the structure of whole-cut meat, practical challenges are yet to be overcome; such as: making a large-scale homogenous porous scaffold; seeding the cells uniformly on 3D scaffold; and rapid and easy vascularization.

“Frost, H. K., Andersson, T., Johansson, S. et al “Electrospun nerve guide conduits have the potential to bridge peripheral nerve injuries in vivo”. Sci Rep 8, 16716 (2018). https://doi.org/10.1038/s41598-018-34699-8” discloses an electrospun polycaprolactone (PCL) construct having a below micron sized pores in the bulk of the construct with channels extending through the construct. The construct is rolled and used for culturing nerve guides inside the channels and the bulk of the scaffold to maintain nutrients and oxygen by capillary suction of the media.

“Baranski, J. D.; Chaturvedi, R. R.; Stevens, K. R.; Eyckmans, J.; Carvalho, B.; Solorzano, R. D.; Yang, M. T.; Miller, J. S.; Bhatia, S. N.; Chen, C. S. Geometric control of vascular networks to enhance engineered tissue integration and function. Proc. Natl. Acad. Sci. U.S.A 2013, 110, 7586.” discloses a construct made from PDMS with a pattern of parallel grooves produced by moulding. Cord-like patterns of endothelial cells encapsulated in fibrin were cultured inside the grooves. The construct was used in a conventional 2D culturing process.

As such, there still remains the need for a construct suitable for PFB cell culturing of muscles that is easily and efficiently seeded, cheap to produce and allows for the culture of high density of cells.

There is also a need for a system and methods that utilise such constructs that can produce muscles cell at high densities that can provide a meat analogue at portion size with reduced costs, greater energy efficacy and speed.

### SUMMARY OF INVENTION

The aforementioned bottlenecks of the PFBs are addressed by the current invention which, inter alia, provides a construct (also referred to as Cellular Agriculture Spiral-wound Pseudovascularised (CASP) construct) and a bioreactor that utilises such a construct to culture cells, such as muscle cells. The current invention provides the possibility of increasing the size of 3D cultured tissue construct, thus taking a step towards producing a portioned-sized 3D piece of cultured meat (i.e. meat analogue). The current invention may provide fast, simple, controllable and scalable constructs and methods of using such constructs, to create uniform pseudo-vascularized spiral-wound scaffolds homogenously seeded with muscle cells.

In one aspect of the invention there is provided a construct for perfusion bioreactor cell culture comprising:
at least one flexible polymer sheet for rolling or folding in use; and
a plurality of channels for transport of cell culture medium to cells and/or hosting cells in use.

In some embodiments, the construct is suitable for cell proliferation, cell differentiation and/or harvesting of cells.

In some embodiments, the at least one flexible polymer sheet has a Young's modulus of at most 500 MPa. In some embodiments, the at least one flexible polymer sheet has a Young's modulus from 10 MPa to 500 MPa. In some embodiments, the at least one flexible polymer sheet has a Young's modulus of at most 400 MPa.

In some embodiments, the at least one flexible polymer sheet has an elastic area (or region) of at least 1% of the total length of polymer sheet. In some embodiments, the at least one flexible polymer sheet has an elastic area (or region) from 1% of the total length of polymer sheet to 15% of the total length of polymer sheet. In some embodiments, the at least one flexible polymer sheet has an elastic area (or region) from 1% of the total length of polymer sheet to 10% of the total length of polymer sheet.

In some embodiments, the at least one flexible polymer sheet is non-porous or comprises an average pore diameter from 0 to 49 μm and the channels are open channels. In some embodiments, the at least one flexible polymer sheet is non-porous. In some embodiments, the at least one flexible polymer sheet comprises an average pore diameter from 0 to 49 μm. In some embodiments, the channels are open channels. In some embodiments, the at least one non-porous flexible polymer sheet comprises a Young's modulus of around 280 MPa. In some embodiments, the at least one non-porous flexible polymer sheet comprises a Young's modulus from 100 to 300 MPa. In some embodiments, the at least one non-porous flexible polymer has an elastic area (or region) of around 5% of the total length of polymer sheet. In some embodiments, the at least one non-porous flexible polymer sheet has an elastic area (or region) from 1% of the total length of polymer sheet to 10% of the total length of polymer sheet.

Thus in another aspect there is provided a construct for perfusion bioreactor cell culture comprising:
at least one flexible polymer sheet for rolling or folding in use, wherein the at least one flexible polymer sheet is non-porous or comprises an average pore diameter from 0 to 49 μm; and
a plurality of channels for transport of cell culture medium to cells and/or hosting cells in use, wherein the channels are open channels.

In some embodiments, each of the plurality of open channels comprise an average width from 20 μm to 1000 μm. In some embodiments, each of the plurality of open channels comprise an average width from 50 μm to 700 μm.

In some embodiments, the average pore diameter is less than 20 μm. In some embodiments, the average pore diameter is less than 10 μm. In some embodiments, the average pore diameter is from 0 to 20 μm. In some embodiments, the average pore diameter is from 0 to 10 μm.

In some embodiments, each of the plurality of open channels comprise an average depth from 50 μm to 500 μm. In some embodiments, each of the plurality of open channels comprise an average depth from 100 μm to 500 μm.

In some embodiments, the construct comprises a laminate structure, wherein the at least one flexible polymer sheet comprises a first flexible polymer sheet and a second flexible polymer sheet adhered to each other, and wherein the first or second flexible polymer sheet comprises the plurality of open channels.

In some embodiments, the first flexible polymer sheet and the second flexible polymer sheet can be adhered to each other using a partial acid/base solubilisation of surface moieties. This can be followed by drying (for example freeze drying, oven drying, vacuum drying, or similar) to produce an insoluble protein/polymer porous network. In some embodiments, the first flexible polymer sheet and the second flexible polymer sheet can be separated by a bean curd sheet layer using citric, hydrochloric, phosphoric, sulphuric or other organic or mineral acids. In some embodiments, the first flexible polymer sheet and the second flexible polymer sheet can be adhered to each other using an enzymatic reaction of surface proteins/amino-acids (e.g. transglutaminase) and optionally dried to form insoluble porous network (e.g. aerogel). In some embodiments, the first flexible polymer sheet and the second flexible polymer sheet can be adhered to each other using calcium or other divalent ion bridging to support gel formation and final hydrogel or dried hydrogel/aerogel. In some embodiments, the first flexible polymer sheet and the second flexible polymer sheet can be adhered to each other using a combination of the above.

In some embodiments, the first and second flexible polymer sheets can be co-formed, for example by co-extrusion, high/low moisture extrusion, or by film/layer wet casting/coating. Such processes can result in a controlled porosity and layer thickness.

In some embodiments, the at least one flexible polymer sheet comprises an average thickness from 30 μm to 1000 μm.

In some embodiments, the at least one flexible polymer sheet comprises a porous polymer comprising pores of an average diameter of 100 μm to 600 μm and wherein the channels are closed channels. In some embodiments, the at least one porous flexible polymer sheet comprises a Young's modulus of around 110 MPa. In some embodiments, the at least one porous flexible polymer sheet comprises a Young's modulus from 100 to 120 MPa. In some embodiments, the at least one porous flexible polymer has an elastic area (or region) from about 1.4% of the total length of polymer sheet to about 3% of the total length of polymer sheet.

Thus in another aspect there is provided, a construct for perfusion bioreactor cell culture comprising:
at least one flexible polymer sheet for rolling or folding in use, wherein the at least one flexible polymer sheet comprises a porous polymer comprising pores of an average diameter of 100 μm to 600 μm and wherein the channels are closed channels; and
a plurality of channels for transport of cell culture medium to cells and/or hosting cells in use, wherein the channels are closed channels.

In some embodiments, each of the plurality of closed channels are hollow; or
each of the plurality of closed channels comprise a channel forming member disposed therein.

In some embodiments, each of the plurality of closed channels are hollow.

In some embodiments, each of the plurality of closed channels comprise a channel forming member disposed therein.

In some embodiments, each of the plurality of closed channels have an average width from 50 μm to 1000 μm.

In some embodiments, the porous polymer has an average thickness from 100 μm to 1000 μm.

In some embodiments, each of the plurality channels are spaced apart by an average distance from 100 μm to 1500 μm. In some embodiments, each of the plurality of open channels are spaced apart by an average distance from 100 μm to 1500 μm. In some embodiments, each of the plurality of closed channels are spaced apart by an average distance from 100 μm to 1500 μm.

In some embodiments, the channels extend longwise from one surface of the flexible polymer sheet to an opposing surface of the flexible polymer sheet. In some embodiments, the open channels extend longwise from one surface of the flexible polymer sheet to an opposing surface of the flexible polymer sheet. In some embodiments, the closed channels extend longwise from one surface of the flexible polymer sheet to an opposing surface of the flexible polymer sheet.

In some embodiments, the channels are substantially parallelly aligned. In some embodiments, the open channels are substantially parallelly aligned. In some embodiments, the closed channels are substantially parallelly aligned.

In some embodiments, the each of the channels comprises a uniform cross-section. In some embodiments, the each of the open channels comprises a uniform cross-section. In some embodiments, the each of the closed channels comprises a uniform cross-section.

In some embodiments, the at least one flexible polymer sheet comprises a biodegradable polymer.

In some embodiments, the at least one flexible polymer sheet comprises an edible polymer.

In some embodiments, the edible polymer may be a textured vegetable protein sheet, textured vegetable protein particles, or textured vegetable protein chunks. In some embodiments, the edible polymer may be tofu (soft/firm), for example frozen tofu. The tofu may be moulded. In some examples, the edible polymer may be a solidified rehydrated soy protein isolate.

In some embodiments, the edible polymer may be blended or coated with a cell adherent material, for example collagen, gelatine, or fibrin.

In some embodiments, the at least one flexible polymer sheet comprises a digestible polymer.

In some embodiments, the at least one flexible polymer sheet comprises bean curd sheet (BCS). In some embodiments, the BCS is wet BCS. For example, BCS that has been hydrated with a liquid. This may help improve the mechanical properties of the BCS. In some embodiments, the at least one flexible polymer sheet comprises BCS and comprises a Young's modulus of about 38 MPa. In some embodiments, the at least one flexible polymer sheet comprises BCS and comprises a Young's modulus from about 20 MPa to about 50 MPa. In some embodiments, the at least one flexible polymer sheet comprises BCS and comprises a Young's modulus of at most 100 MPa. In some embodiments, the at least one flexible polymer sheet comprises BCS and comprises an elastic area of region of at least 5% of the total length of the flexible polymer sheet. In some embodiments, the at least one flexible polymer sheet comprises BCS and comprises an elastic area of region of at from 5% of the total length of the flexible polymer sheet to 15% of the total length of the flexible polymer sheet. In some embodiments, the at least one flexible polymer sheet comprises BCS and comprises an elastic area of region of at from 7% of the total length of the flexible polymer sheet to 10% of the total length of the flexible polymer sheet.

In some embodiments the BCS comprises the plurality of channels are formed in a surface of the BCS, for example by etching (e.g., laser etching), or moulding (e.g., wet moulding), or embossing, or freeze casting. In some embodiments, the BCS is coated. In particular, the BCS may be coated with a cell adherent coating or a firming agent. The cell adherent coating may support cell attachment and proliferation. In examples, the coating may comprise one or more of: gelatin, collagen, fibrin, calcium chloride, or protein isolate (e.g., soy protein isolate or pea protein isolate). In some embodiments, the coating comprises a blend of gelatin, soy protein isolate, and calcium chloride. The blend may be a solution that is heated and sprayed or otherwise applied to the BCS to coat the BCS, including the plurality of channels. In some embodiments, the coating comprises a blend of collagen, soy protein isolate, and calcium chloride. In some embodiments, the coating comprises a blend of fibrin, soy protein isolate, and calcium chloride. In some embodiments, the coating comprises a blend of gelatin, pea protein isolate, and calcium chloride. In some embodiments, the coating comprises a blend of collagen, pea protein isolate, and calcium chloride. In some embodiments, the coating comprises a blend of fibrin, pea protein isolate, and calcium chloride.

In some embodiments, a method of forming a construct comprises:
providing a bean curd sheet (BCS) having a plurality of channels formed in a surface of the BCS, and
coating the surface of the BCS that comprises the plurality channels.

In some embodiments, the coating comprises a cell adherent coating.

In some embodiments, the at least one flexible polymer sheet may comprise a BCS backing layer and a plurality of formations attached to the BCS backing layer and defining the plurality of channels between the plurality of formations. In embodiments, the plurality of formations may comprise gelatin, collagen, fibrin, calcium chloride, or protein isolate (e.g., soy protein isolate or pea protein isolate). In some embodiments, the plurality of formations may comprise a blend of gelatin, soy protein isolate, and calcium chloride. In some embodiments, the plurality of formations may comprise a blend of collagen, soy protein isolate, and calcium chloride. In some embodiments, the plurality of formations may comprise a blend of fibrin, soy protein isolate, and calcium chloride. In some embodiments, the plurality of formations may comprise a blend of gelatin, pea protein isolate, and calcium chloride. In some embodiments, the plurality of formations may comprise a blend of collagen, pea protein isolate, and calcium chloride. In some embodiments, the plurality of formations may comprise a blend of fibrin, pea protein isolate, and calcium chloride.

In some embodiments, the BCS backing layer is coated with a hardening coating. The plurality of formations can be attached to the hardening coating. In some embodiments, the hardening coating may comprise a transglutaminase (TGase). In some embodiments, the hardening coating may comprise 1-ethyl-3-(3-dimethyl aminopropyl) carbodiimide (EDC). In some embodiments, the hardening coating may comprise a N-hydroxy succinimide (NHS). In some embodiments, the hardening coating may comprise a combination of EDC and NHS. In particular, the hardening coating may comprise a mix of 25 mM of EDC and 10 mM of NHS in 100 mL of a mixture of ethanol/distilled water (9:1 v/v). The hardening coating may act to crosslink or couple or cure or harden amines to improve surface structure.

In some embodiments, the plurality of formations are formed by moulding. For example, a moulded sponge layer may be used to mould the plurality of formations on the BCS backing layer (and optionally on the transglutaminase coating). In some embodiments, the plurality of formations are formed by pouring a heated material onto the BCS (and optional transglutaminase coating), then pressing the moulded sponge layer onto the heated material to mould the heated material into the plurality of formations. In examples, the assembly of the heated material be gelled with the moulded sponge layer in situ. In examples, the assembly may be maintained at about 4 degrees Celsius for about 1 hour to gel the plurality of formations. After gelling, the moulded sponge layer may be removed.

The resultant construct may then be frozen at −20 degrees Celsius. The construct may alternatively or additionally be freeze-dried. Before or after freezing and/or freeze-drying the construct can be rolled or folded. The construct may then then rinsed using sterile culture media, and/or phosphate-buffered saline (PBS), and/or ethanol. After rinsing a concentrated cell pellet may be applied to the surface, for example spread over the surface. The construct can be immersed in media for proliferation of the cells.

In some embodiments, a method of forming a construct comprises:
providing a bean curd sheet (BCS) as a backing layer, and
moulding a plurality of formations onto the BCS backing layer, the plurality of formations being moulded to define a plurality of channels extending between the plurality of formations.

In some embodiments, the method may further comprise coating the BCS backing layer with a hardening coating before moulding the plurality of formations. The hardening coating may comprise one or more of: TGase, EDC, and NHS as set out above.

In some embodiments, the method of moulding the plurality of formations may comprise applying a heated material to the BCS backing layer and then pressing a moulded sponge layer onto the BCS backing layer to mould the heated material. In examples, the heated material may comprise one or more of: gelatin, collagen, fibrin, calcium chloride, or protein isolate (e.g., soy protein isolate or pea protein isolate).

The method may further comprise gelling and/or freezing the heated material to provide the plurality of formations. The method may further comprise rinsing the construct. The construct may be rinsed with sterile culture media, and/or PBS and/or ethanol.

In some embodiments, the at least one flexible polymer sheet comprises a polymer selected from at least one of polycaprolactone, polypropylene and/or polystyrene.

In another aspect of the present invention there is provided a method of forming a construct for perfusion bioreactor cell culture, the method comprising the steps of:
dissolving a polymer in a solvent to form a mixture;
mixing a porogen having a diameter of at most 125 μm into the mixture;
applying the mixture onto a planar structure;
immersing the planar structure comprising the applied mixture in an anti-solvent;
and drying the planar structure comprising the applied mixture to form a porous flexible polymer sheet for rolling or folding in use;
wherein the planar structure comprises a plurality of channel forming members and wherein the mixture is applied so that the mixture encases the channel forming members; and
removing the construct comprising the channel forming members therein from the planar structure.

In some embodiments, the channel forming members of the planar structure comprise a plurality of wires, optionally wherein the wires comprise an average diameter from 5 μm to 1000 μm, further optionally wherein the wires comprise an average diameter of between 270 μm to 340 μm. In some embodiments, the plurality of wires, comprise an average diameter from 20 μm to 100 μm. In some embodiments, the plurality of wires, comprise an average diameter from 70 μm to 100 μm.

In some embodiments, the method further comprises a step of removing the channel forming members from the construct to provide closed channels.

In some embodiments, each of the plurality of channel forming members are spaced apart by an average distance of between 100 μm and 1500 μm.

In some embodiments, the solvent comprises acetone.

In some embodiments, the porogen comprises salt particles. In some embodiments, the porogen comprise sodium chloride (NaCl). In some embodiments, the porogen comprises polyethylene glycol (PEG.

In some embodiments, the porous flexible polymer sheet comprises pores of an average pore diameter of 100 μm to 600 μm

In some embodiments, the porous flexible polymer sheet has an average thickness of between 300 μm and 1000 μm.

In another aspect of the present invention there is provided, a method of forming a construct for perfusion bioreactor cell culture, the method comprising the steps of:
providing at least one flexible polymer sheet for rolling or folding in use;
etching a plurality of channels onto at least one surface of the at least one flexible polymer sheet, wherein each channel extends from one surface of the at least one flexible polymer sheet to an opposing surface of the flexible polymer sheet and wherein each of the channels are open channels.

In some embodiments, the channels extend through the at least one surface of the at least one flexible polymer sheet and the method further comprises:
adhering the at least one flexible polymer sheet comprising the plurality of channels extending therethrough to a second flexible polymer sheet to form a laminate structure.

In some embodiments, the plurality of channels are etched onto the at least one surface and extend between two opposing surfaces of the at least one flexible polymer sheet and the method further comprises cutting the at least one flexible polymer sheet or the laminate structure so each channel extends from one surface of the at least one flexible polymer sheet to an opposing surface of the flexible polymer sheet.

In some embodiments, the plurality of channels are etched onto the at least one surface and extend between two opposing surfaces of the at least one flexible polymer sheet and the method further comprises cutting the at least one flexible polymer sheet so each channel extends from one surface of the at least one flexible polymer sheet to an opposing surface of the flexible polymer sheet.

In some embodiments, the plurality of channels are etched onto the at least one surface and extend between two opposing surfaces of the at least one flexible polymer sheet and the method further comprises cutting the laminate structure so each channel extends from one surface of the at least one flexible polymer sheet to an opposing surface of the flexible polymer sheet.

In some embodiments, the at least one flexible polymer sheet and second flexible polymer sheet are adhered by a flexible polymer.

In some embodiments, each of the plurality channels are spaced apart by an average distance of between 100 μm and 1500 μm.

In some embodiments, the at least one flexible polymer sheet and/or the second flexible polymer sheet comprises an average thickness from 30 μm to 1000 μm. In some embodiments, the at least one flexible polymer sheet comprises an average thickness from 30 μm to 1000 μm. In some embodiments, the second flexible polymer sheet comprises an average thickness from 30 μm to 1000 μm.

In some embodiments, the at least one flexible polymer sheet comprises an average pore diameter from 0 to 49 μm. In some embodiments, the average pore diameter is less than 20 μm. In some embodiments, the average pore diameter is less than 10 μm. In some embodiments, the average pore diameter is from 0 to 20 μm. In some embodiments, the average pore diameter is from 0 to 10 μm.

In some embodiments, each of the plurality of channels comprise an average width from 20 μm to 1000 μm. In some embodiments, each of the plurality of channels comprise an average width from 20 μm to 700 μm. In some embodiments, each of the plurality of channels comprise an average width from 50 μm to 700 μm.

In some embodiments, each of the plurality channels comprise an average depth from 50 μm to 500 μm. In some embodiments, each of the plurality channels comprise an average depth from 100 μm to 500 μm.

In some embodiments, the channel forming members or channels are substantially parallelly aligned. In some embodiments, the channel forming members are substantially parallelly aligned.

In some embodiments, the porous flexible polymer sheet or the at least one flexible polymer sheet and/or second flexible polymer sheet comprise a biodegradable polymer.

In some embodiments, the porous flexible polymer sheet or the at least one flexible polymer sheet and/or second flexible polymer sheet comprise an edible polymer.

In some embodiments, the porous flexible polymer sheet or the at least one flexible polymer sheet and/or second flexible polymer sheet comprise an digestible polymer.

In some embodiments, the porous flexible polymer sheet or the at least one flexible polymer sheet and/or second flexible polymer sheet comprise a polymer selected from at least one of polycaprolactone, polypropylene and/or polystyrene.

In some embodiments, the at least one flexible polymer sheet comprises bean curd sheet (BCS).

In some embodiments, the method further comprises:
immersing the at least one flexible polymer sheet or the laminate structure in a firming agent.

In some embodiments, the firming agent comprises a food grade firming agent. In some embodiments, the firming agent comprises calcium chloride aqueous solution.

In another aspect of the present invention there is provided, a construct obtainable by a method as described herein.

In another aspect of the present invention there is provided, a system for perfusion bioreactor cell culture comprising:
a chamber having an internal space comprising an internal width;
a construct as described herein, comprising cells seeded thereon,
wherein the construct is disposed within the internal space of the chamber, and wherein the construct is configured as a filled 3-dimensional structure; and
a system for perfusing culture media through the chamber and/or the construct.

In some embodiments, the construct has a width substantially the same as the internal width of the chamber.

In some embodiments, the system for perfusing media comprises a pumping system.

In some embodiments, the filled 3-dimensional structure is a cylindrical structure.

In another aspect of the present invention there is provided, a method of culturing cells comprising the steps of:
applying a cell support agent to a construct as described herein;
seeding cells on the construct;
subjecting the construct to conditions suitable to crosslink the cell support agent;
when the construct comprises channel forming members, removing the channel forming members;
manipulating the construct to form a filled 3-dimensional structure;
disposing the manipulated construct into a chamber of a system for perfusion bioreactor cell culture; and
perfusing culture media through the system and maintaining the manipulated construct under conditions suitable for culturing the cells and culturing the cells thereon or therein.

In some embodiments, the system for perfusion bioreactor comprises a chamber having an internal space comprising an internal width and wherein the construct has a width substantially the same as the internal width of the chamber.

In some embodiments, the method further comprises after step f):
removing the construct from the chamber and applying a cell support agent-specific enzyme to the construct for releasing the cells from the construct; and
recovering the cells.

In some embodiments, the recovered cells are formed into a comestible product.

In some embodiments, the cell support agent comprises fibrin. In some embodiments, the cell support agent-specific enzyme comprises nattokinase. In some embodiments, the cell support agent-specific enzyme comprises food grade nattokinase.

In some embodiments, the 3-dimensional structure is a cylindrical structure.

In some embodiments, culturing the cells comprises cell proliferation and/or cell differentiation, optionally wherein perfusing culture media comprises perfusing a first culture media for cell proliferation and/or perfusing a second culture media for cell differentiation. In some embodiments, culturing the cells comprises cell proliferation. In some embodiments, culturing the cells comprises cell differentiation. In some embodiments, perfusing culture media comprises perfusing a first culture media for cell proliferation and/or perfusing a second culture media for cell differentiation. In some embodiments, perfusing culture media comprises perfusing a first culture media for cell proliferation. In some embodiments, perfusing culture media comprises perfusing a second culture media for cell differentiation.

In some embodiments, the construct an edible construct and the construct is removed from the chamber and the construct and the cells cultured thereon are formed into a comestible product.

In some embodiments, the comestible product is a meat analogue.

In some embodiments, manipulating the construct comprises rolling the construct. For example, rolling the construct into a filled cylinder.

In some embodiments, the construct has open channels and wherein the cells are seeded within the plurality of channels.

In some embodiments, the construct comprises a porous flexible polymer and has closed channels and the cells are seeded within the pores of the porous polymer.

In some embodiments, the cells comprise muscle cells.

Also provided in another aspect is a comestible product obtainable by a method of culturing an edible scaffold as described herein. For example, a comestible product comprising an edible scaffold as described herein and cells cultured thereon.

### BRIEF DESCRIPTION OF FIGURES

FIG. 1 shows a schematic of the process of fabricating improved CASP by using laser etching and sheet binding; a) etching grooves on a flexible polymeric sheet. These grooves can optionally extend through the thickness of the sheet (i.e. forming slits). If the grooves are not cut through, the etched flexible polymeric sheet can be used as a scaffold. b) If the grooves do not extend through the flexible polymeric sheet, the etched flexible polymeric sheet is bound on a second sheet of flexible polymer (B-2). Cutting the ends of these grooves provides open channels (B-3).

FIG. 2 shows an overview of cell culturing using a construct as described herein; As shown the cells and the cell support agent (e.g. fibrin) are added to the scaffold. 2. the fibrin and cells penetrate into pores of construct. After the cell support agent has crosslinked the channel forming members (e.g. wires) are removed. As the hydrogel is crosslinked the cell seeding media and the cells do not enter into the channels. 3. The seeded construct (now with open ended closed channels) is rolled into a full cylinder. 4. the rolled construct is transferred into a cylindrical chamber of a perfusion bioreactor. 5. The perfusion of cell culture media through the construct maintains the cells and leads to cell expansion thus increasing the number of cells. The increase in cell numbers can be observed by comparing staining microscopy images of 1 and 6.

FIG. 3 illustrates physical/geometrical/morphological properties of a PCL scaffold. a) shows a light microscopy image of the rolled scaffold. Open channels can be seen. b) shows a light microscopy image of the porous PCL sheet. Intact channels can be seen under the porous layer. Diameter of the channels and their intervals can be seen and measured. c) shows an SEM image showing the open ends of the channels and hence their desired capability of allowing perfusion (porogens were not used in these sheets for better demonstration of the channels). d) shows intact channels in a non-porous sheet. e) shows a longitudinal cross-section of the channels showing several holes which support media diffusion. The major parts are channel walls with very small pores which is favourable for protecting cells from the sheer stress. f) shows a larger magnification of the channel wall to show different-sized microvoids and morphology of the walls. g) shows an SEM image of the glass-side surface of the scaffold showing pores and their interconnections. Since SEM images don't show transparency, the channels beneath the porous surface are hardly visible. They are shown by rectangles. The bold arrows point to deep holes (darker than usual pores, which shows there is a channel beneath the surface). Lighter arrows show the interconnection between the channels and the bulk of the porous scaffold and demonstrate the fluid flow. h) shows morphology and porosity of the air-side surface of the scaffold;

FIG. 4 illustrates a comparison between seeding efficiencies. a) shows live-dead staining of cells seeded on flatsheet (1 cm*1 cm). b) shows live-dead staining of cells seeded on long (10 cm×1 cm) scaffold (stained after rolling and unrolling) as can be seen, the rolling process does not impact the seeding and viability of the cells. c, d) shows a schematic comparison of cell distribution between static cell seeding (d) and the proposed rolling sheet method (c); the static cell seeding cannot provide uniform spatial cell distribution, while the sheet rolling offers a homogenous distribution.

FIG. 5 illustrates proliferation of C2C12s on flat-sheet PCL scaffolds, initially seeded with 50,000 cells/cm², cultured for 5 days. a) shows the cells successfully seeded on the scaffold initially after the seeding process (500 μm scale bar). b to f) show cells on scaffolds respectively from day 1 to day 5 of proliferation (live cells are stained green while dead cells can be detected in red; 500 scale bars). g) shows number of cells vs. days of proliferation. Cells were counted by using staining and imaging and also using a cell-counter as methods. This graph shows number of the cells counted for samples seeded on a 1 cm×1 cm scaffold with initial seeding density of 50,000 cells/cm² (n=2, N=3). This graph shows the cell proliferation and helps detecting the exponential growth phase period. The orange line shows that proliferation doesn't follow the exponential trend after day 4. As a result, the exponential growth phase was detected from day 0 to day 4. h) shows a logarithmic graph that calculates the doubling time in the exponential growth phase (R² of the trendline is 0.996 and 0.7604 is the slope of the trendline);

FIG. 6 illustrates proliferation trends of rolled PCL scaffolds seeded with 50,000 cells/cm². a) shows the cells successfully seeded on the scaffold after the scaffold being rolled and un-rolled once (750 μm scale bar). b to f) show cells on scaffolds respectively from day 1 to day 5 of proliferation (live cells are stained green while dead cells can be detected in red; 750 scale bars). g) shows number of cells vs. days of proliferation. Cells were counted by using staining and imaging and also using a cell-counter as methods. This graph shows mean number of the cells counted on 1 cm×1 cm samples from different places of a rolled scaffold cultured in PFBs (n=1, N=2). h) Shows a logarithmic graph that calculates the doubling time in the exponential growth phase. Here the exponential growth phase were detected to be from day 1 to day 5 (As the trendline containing day zero could not fit the exponential trend very well; i.e. R² of 0.96. Here the R² of the trendline is 0.985 and 0.815 is the slope of the trendline);

FIG. 7 illustrates static and dynamic cell culture in PFBs. a) shows an array of twelve staining images showing cell proliferation and viability in static cell culture (40 mm high, 8 mm in diameter scaffold, seeded initially by 50,000 cells/cm²). Necrotic middle and outlet parts are seen by prevalent numbers of red (dead) cells. On the other hand, b) shows live/dead staining of the scaffold with the exact same properties inside a PFB (dynamic culture) and demonstrates a uniform cell growth and viability. Scale bars are 500 μm for all images;

FIG. 8 illustrates qualitative demonstration of scalability of the perfusion bioreactors (PFBs). a) shows rolled scaffolds with 3 different diameters (8, 13 and 23 mm from left to right) each cultured for 3 days inside a PFB. This provides a proof of concept to show the scalability of the culturing using the scaffolds. b) depicts the sampling strategy to obtain samples from inner layers, middle layers and outer layers of each scaffold to do the comparison. c to e) are respectively samples from inner, middle and outer layers of the biggest scaffold (scale bars are 300 μm). f to h) are respectively samples from inner, middle and outer layers of the medium size scaffold (scale bars are 300 μm); and i to k) show respectively samples from inner, middle and outer layers of the small scaffold (scale bars are 300 μm). All the scales are able to maintain cell growth in all layers. L to n) show the bioreactors used to culture these scale spectrum of scaffolds;

FIG. 9 illustrates a comparison on oxygen consumption trends between two different scales of PFBs with the similar perfusion length and flowrate per single channel (but different diameters). a) shows the dissolved oxygen in inlet (●) and outlet (▴) of reactors with diameter of 8 mm and height of 10 mm. b) shows similarly dissolved oxygen data for a reactor with the same height but scaled diameter of 13 mm. Flowrate per single channel was 0.5 μL min⁻¹ for both scales to make comparison possible; the oxygen use was found to be similar as it was expected.

FIG. 10 illustrates differentiation in PFB after 7 days. a) shows FDA staining of samples taken from reactors (5 mm height and 8 mm diameter which were gone through 5 days of proliferation (initial cell seeding of 50,000 cells/cm²) followed by 7 days of differentiation. some levels of alignment along the channels can be seen. b) showing myoblasts fusion in PFBs after 3 days of proliferation (not gone through differentiation yet). c) depicts some mature myotubes in samples differentiated as in (a). Finally, d) demonstrates cell alignment along the perfusion channels (a higher focus of a);

FIG. 11 shows laser etched polypropylene/polystyrene (pp/ps) non-woven sheets. Laser etching up to 150 μm accuracy was possible in a rapid scalable manner;

FIGS. 12 (a and b) shows stained cells on an etched polymeric construct. FDA stain the cytoplasm of the C2C12 cells in green and shows the parallel structure of the differentiated cells. Striation of differentiated myotubes on etched pp/ps sheets (4 days of proliferation followed by 6 days of culture in differentiation media) was observed. Growing C2C12s on channel widths between 150 to 400 μm leads improved differentiation:

FIG. 13 shows laser etched bean curd sheet (BCS). Modification of the CNC laser (frequency, strength of the laser beam and micromotor speed) was necessary for etching the BCS and not burning it.

FIG. 14 shows a cleanly etched sheet of bean curd performed rapidly and in a scalable way by laser.

FIG. 14 shows day 3 of proliferation on BCS+ fibrin/BCS without hydrogel, BCS with fibrin after seeding, and BCS with gelatin. a) cells growing inside a fibrin layer mounted on laser etched BCS. The grooves are clear and qualitatively and in comparison to the other conditions the best proliferation conditions are seen. b) shows a very limited number of cells attached to the surface of BCS without any hydrogel present (plain BCS). C) shows cells growing inside fibrin mounted on a non-etched BCS. The growth of cells was less than a); also it is noticeable that the cells are not as organised as case a). d) shows cells can attach to BCS modified by gelatin (attachment to a less extent compared to fibrin cases).

FIG. 15 shows laser etched BCS after modification by a firming agent and sterilization by 70% ethanol aqueous solution and 10 days in 37° C. culture media. This shows that long contact to aqueous media does not impact the integrity of the construct;

FIG. 16 shows the profile of an etched BCS sheet after firming, sterilization and long contact with liquid culture media. The width of the grooves are within 200 μm desired range;

FIG. 17 shows the crossectional profile of an etched BCS sheet showing that the depth of the grooves are around 140 μm;

FIGS. 18 (a and b) shows SEM images of the thickness cross-section of BCS (a with higher magnification). The slight porosity of these digestible polymers and their thickness (120-140 μm) was measured;

FIG. 19 (a to c) shows SEM images of the BCS surface after firming and sterilisation. It shows the very small pore scale of the BCS (around ˜1 μm);

FIG. 20 shows a schematic of a 200 μm groove on a BCS (seeded by 5000 cells/cm²). Filled squares represent cells (C). Their number increases day by day (proliferation), until the almost cover the whole channel by day 5. Differentiation after 6 days turns the individual myotubes into 3 thick continues myofibers (the sizes of myofibers are also in line with experimental data (40-60 μm width);

FIG. 21 shows Hoechst and FDA staining of the cells showing their qualitative expansion through proliferation (a: day 2, b: day 3, c: day 4). It is noticeable that cell numbers have increased day by day;

FIG. 22 shows a section of a channel (i.e. a groove) on a BCS scaffold after 4 days of proliferation and 6 days of differentiation. Similar to FIG. 21 (after differentiation), an array of parallel myotubes is detected (3-4 parallel myotubes);

FIG. 23 shows a macroscopic version of a BCS construct (cells differentiated on BCS). The texture and the colour of the product resembles meat;

FIG. 24 shows an example of a construct having a BCS backing layer and moulded formations defining a plurality of channels;

FIG. 25(a) shows a photograph of a coated BCS construct;

FIG. 25(b) shows a confirmed healthy culture on the BCS construct after 4 days;

FIG. 26 shows a force-displacement graph for a nonwoven polypropylene (Novatexx 2471) construct;

FIG. 27 shows a force-displacement graph for a non-porous PCL construct (PCL (8% (w/V) in acetone; no porogens; casted sheet);

FIG. 28 shows a force-displacement graph for porous sheets of PCL construct;

FIG. 29 shows a force-displacement graph for dry BCS; and

FIG. 30 shows a force-displacement graph for wet BCS.

### DETAILED DESCRIPTION

The present invention relates to constructs for cell culture. The constructs include at least one flexible polymer sheet.

The flexible polymer sheet includes a polymer material. Polymers are a series of monomer groups linked together. Polymers that may be used to form the flexible polymer sheet may be any polymer suitable for culturing and/or maintenance of cells. Suitable polymers include biodegradable polymer. The polymer may be a biocompatible polymer. Biodegradable polymers are any polymers that may be broken down by biological systems, such as polymers that can be broken down into harmless products by the action of living organisms. Biocompatible polymers are, along with any metabolites or degradation products thereof, generally nontoxic to cells or to a recipient (such as a human or animal), and do not cause any significant adverse effects to cells or a recipient, at concentrations resulting from the degradation of the polymers. Generally speaking, biocompatible polymers are polymers that do not elicit result in negative effects on cell health or in a recipient. As one use for the constructs described herein is the production of comestible products, biocompatible and/or biodegradable polymers may be advantageous if the scaffold or part thereof is consumed (for example ingested) by a person.

Biodegradables polymers include linear aliphatic polyesters such as polylactic acid, polyglycolic acid, polycaprolactone, polyhydroxybutyrate, polyhydroxyvalerate and their copolymers within the aliphatic polyester family such as poly(lactic-co-glycolic acid) and poly(glycolic acid-co-caprolactone); copolymers of linear aliphatic polyesters and other polymers such as poly(glycolic acid-co-trimethylene carbonate) copolymers, poly(lactic acid-co-lysine) copolymers, tyrosine-based polyarylates or polyiminocarbonates or polycarbonates, poly(lactide-urethane) and poly(ester-amide) polymers; polyanhydrides such as poly(sebacic anhydride); polyorthoesters such as 3,9-diethyidiene-2,4,8,10-tetraoxaspiro-5,5-undecane based polymers; poly(ester-ether) such as poly-p-dioxanone; polyamides, poly(amide-enamines) and poly(amido amine) dendrimers; and phosphorus-based polymers such as polyphosphazene and poly[bis(carboxy-lactophenoxy) phosphazene.

The polymer may be an aliphatic polyester such as polycaprolactone, polyesteramides, modified polyethylene terephthalate, polylactic acid (PLA) and its copolymers, terpolymers based on polylactic acid, polyglycolic acid, polyalkylene carbonates (such as polyethylene carbonate), polyhydroxyalkanoates (PHA), poly-3-hydroxybutyrate (PHB), poly-3-hydroxyvalerate (PHV), poly-3-hydroxybutyrate-co-4-hydroybutyrate, poly-3-hydroxybutyrate-co-3-hydroxyvalerate copolymers (PHBV), poly-3-hydroxybutyrate-co-3-hydroxyhexanoate, poly-3-hydroxybutyrate-co-3-hydroxyoctanoate, poly-3-hydroxybutyrate-co-3-hydroxydecanoate, poly-3-hydroxybutyrate-co-3-hydroxyoctadecanoate, and succinate-based aliphatic polymers (e.g., polybutylene succinate, polybutylene succinate adipate, polyethylene succinate, etc.); aromatic polyesters and modified aromatic polyesters; and aliphatic-aromatic copolyesters.

The polymer may be polycaprolactone (PCL). Polycaprolactone (PCL) is one of the earliest, commercially available, synthetic polymers characterized by a large set of biodegradation and mechanical properties that can be finely controlled by regulating the local environmental (i.e., microorganisms, enzymes, hydrolysis). PCL has relatively fast resorbability and long-term degradation in the presence of water (up to 3 to 4 years. In comparison with other aliphatic polyesters, the rheological and viscoelastic properties render PCL easy to manufacture and manipulate into a wide range of three-dimensional platforms (e.g. porous scaffolds). The durability and the long time-span of PCL biodegradability (up to 2 years) may allow for reuse of a construct. PCL is also comparably cheap in relation to other polymers used in tissue engineering.

The polymers may be edible polymers. Edible polymers refers to any polymer that is acceptable for use in an edible product.

Examples of edible polymers include polyvinyl alcohol, carboxyvinyl polymer, hydroxypropylmethylcellulose, hydroxyethylcellulose, methylcellulose, ethylcellulose, low-substituted hydroxypropylcellulose, crystalline cellulose, carboxymethylcellulose sodium, a synthetic polymer compound such as carboxymethylcellulose calcium, carboxymethylcellulose and carboxymethylstarch sodium, sodium alginate, dextran, casein, pullulan, pectin, guar gum, xanthan gum, tragacanth gum, acacia gum, zein, gelatin, chitin and chitosan, silk, fibrin and polymer compounds obtained from natural products such as starch or soybean.

The use of an edible polymer may provide a construct and product including a construct which is edible. For example, a cell culture grown on the construct may provide for an edible product that does not require removal of the construct before consumption.

The edible polymer may be bean curd sheet (BCS). BCS may also be known as tofu skin, bean curd sheet, soybean curd or bean curd robes. The BCS may be initially in dried form. If in dried form the BCS is rehydrated before use. BCS may be obtained by methods such as industrial curding. Curding is physio-chemical process which comprises protein molecules flocculation due to change in pH and temperature. Milk and soymilk are two of the most well-known products used traditionally for curding. Curding milk will produce cheese; while tofu is obtained by soymilk curding. BCS is a pressed tofu in the form a sheet. Curding can be stimulated by adding enzymes, acids or various salts to the initial liquid. Heating to below boiling point (i.e. simmering) without any mixing or adding additional agents will naturally lead to a layer of curd on top of soymilk. This layer is mechanically separated from the liquid and dried on a flat surface to obtain BCS.

BSC is animal-free, cheap and abundant. Being expensive and hence not scalable are usually drawbacks of many researched tissue culture scaffolds. BCS obtained from soybeans is cheap and as another advantage it is made at industrial scales by known methods. BCS is also elastic and machinable. Machinability may be advantageous in industrial processes using the BCS while elasticity may provide an opportunity for mechanical stimulation (stretching) of the construct. Mechanical stimulation may help provide more efficient methods for differentiating and maturating cells such as muscle cells cultured on a construct including BCS. It has also been found that BCS can be etched (for example by a laser) without the release of inhibitory by-products that may impair or prevent cell culture on the BCS.

In some embodiments, the edible polymer may be a textured vegetable protein sheet, textured vegetable protein particles, or textured vegetable protein chunks. In some embodiments, the edible polymer may be tofu (soft/firm), for example frozen tofu. The tofu may be moulded. In some examples, the edible polymer may be a solidified rehydrated soy protein isolate.

In some embodiments, the edible polymer may be blended or coated with a cell adherent material, for example collagen, gelatine, or fibrin.

The flexible polymer sheet may be a digestible polymer. “Digestible” refers to a material that, when eaten by a subject can be broken down into compounds that can be absorbed and used as nutrients or eliminated by the subject's body. Digestible polymers include BCS, polylactic acids (PLAs), synthetic polyamides, polycarbonates, polyisocyanurates (PIRs), polyurethanes, polyethers, proteins, polysaccharides (such as starches), polylactones, polylactams or glycols.

The flexible polymer sheet may be non-porous. That is to say that the flexible polymer sheet may not have any pores or voids in the substrate of the flexible polymer sheet. Non-porous may refer to a flexible polymer sheet that has a low number of pores. Non-porous may also refer to a flexible polymer sheet that includes pores, however the pores are not permeable to fluids and/or cells under normal conditions for uses as described herein. For example, a non-porous flexible polymer sheet may have pores with an average pore diameter ranging between 0 to 49 μm. For example, a non-porous flexible polymer sheet may have pores with an average pore diameter ranging between 0 to 20 μm. For example, a non-porous flexible polymer sheet may have pores with an average pore diameter ranging between 0 to 10 μm. For example, a non-porous flexible polymer sheet may have a maximum average pore diameter of 20 μm. For example, a non-porous flexible polymer sheet may have a maximum average pore diameter of 10 μm. For example, a non-porous flexible polymer sheet may have a maximum average pore diameter of 49 μm. For example, at most 49 μm, at most 48 μm, at most 47 μm, at most 46 μm, at most 45 μm, at most 44 μm, at most 43 μm, at most 42 μm, at most 41 μm, at most 40 μm, at most 39 μm, at most 38 μm, at most 37 μm, at most 36 μm, at most 35 μm, at most 34 μm, at most 33 μm, at most 32 μm, at most 31 μm, at most 30 μm, at most 29 μm, at most 28 μm, at most 27 μm, at most 26 μm, at most 25 μm, at most 24 μm, at most 23 μm, at most 22 μm, at most 21 μm, at most 20 μm, at most 19 μm, at most 18 μm, at most 17 μm, at most 16 μm, at most 15 μm, at most 14 μm, at most 13, μm at most 12 μm, at most 11 μm, at most 10 μm, at most 9 μm, at most 8 μm, at most 7 μm, at most 6 μm, at most 5 μm, at most 4 μm, at most 3 μm, at most 2 μm, at most 1 μm, or at most 0 μm. An acceptable pore size for a non-porous sheet may be determined by the size of the cells to be cultured thereon. For example, a non-porous polymer sheet may have pores smaller than the size of the cells to be cultured therefore preventing cells penetrating into the bulk of the porous polymer sheet.

The flexible polymer sheet may include a porous polymer. The flexible polymer may be a flexible porous polymer sheet. A porous polymer is used to refer to a structural matrix, which includes a solid region and an open porous region comprising spaces or discontinuities between adjacent areas of the solid region.

Porous polymer sheets may be solid or semi-solid substrates having openings or apertures (pores) which allow cells to partially or completely infiltrate the scaffold. Porous scaffolds may also allow the growth of cells on the surface of the scaffold. Porous polymer sheets may allow for the transport of nutrients, gases and other molecules such as nutrients into and out of the scaffold and ergo through and to the cells cultured on or in the sheet and therefore the construct. Thus the porous polymer sheets described herein act as a 3-dimensional matrix that allow for the culture and maintenance of cells in a 3-dimensional architecture. Examples of porous polymer sheets include a fibrous scaffold with openings or apertures between the fibres of the substrate, a mesh with openings or apertures between the network of structural components that make up the mesh, or a solid substrate with pores throughout the substrate, such as a sponge or foam.

The pores of the porous polymer sheets may have an average pore diameter suitable for allowing infiltration and support cells within the polymer sheet. The pores may have an average diameter between 100 μm to 600 μm. For example, the porous scaffold may have pores with an average pore diameter of 105 μm, 110 μm, 115 μm, 120 μm, 125 μm, 130 μm, 135 μm, 140 μm, 145 μm, 150 μm, 155 μm, 160 μm, 165 μm, 170 μm, 175 μm, 180 μm, 185 μm, 190 μm, 195 μm, 200 μm, 205 μm, 210 μm, 215 μm, 220 μm, 225 μm, 230 μm, 235 μm, 240 μm, 245 μm, 250 μm, 255 μm, 260 μm, 265 μm, 270 μm, 275 μm, 280 μm, 285 μm, 290 μm, 295 μm, 300 μm, 305 μm, 310 μm, 315 μm, 320 μm, 325 μm, 330 μm, 335 μm, 340 μm, 345 μm, 350 μm, 355 μm, 360 μm, 365 μm, 370 μm, 375 μm, 380 μm, 385 μm, 390 μm, 395 μm, 400 μm, 405 μm, 410 μm, 415 μm, 420 μm, 425 μm, 430 μm, 435 μm, 440 μm, 445 μm, 450 μm, 455 μm, 460 μm, 465 μm, 470 μm, 475 μm, 480 μm, 485 μm, 490 μm, 495 μm, 500 μm, 505 μm, 510 μm, 515 μm, 520 μm, 525 μm, 530 μm, 535 μm, 540 μm, 545 μm, 550 μm, 555 μm, 560 μm, 565 μm, 570 μm, 575 μm, 580 μm, 585 μm, 590 μm, 595 μm or 600 μm.

Average pore size may be determined using optical methods such as scanning electron microscopy (SEM), atomic force microscopy (AFM), computed tomography methods and/or transmission electron microscopy (TEM). Other methods that may be used include X-ray refraction methods, imbibition methods, mercury injection methods, and gas expansion methods.

The average pore size and porosity (or density of pores) may affect the penetration of cells into the scaffold and define the spatial distribution of cells within the 3D matrix of the scaffold. In addition, average pore size and porosity may affect the flow resistance, the transportation of nutrients, and/or the excretion of waste products from cells cultured thereon and/or therein.

The flexible porous polymer sheet may have open cell pores. The pores may be connected to each other thus providing a porous matrix with a 3D network of interconnected pores. The flexible porous polymer sheet may have a high surface area provided by the 3-dimensional porous structure.

A sheet refers to a thin continuous piece of material having a high length to thickness ratio and a high width to thickness ratio. As such, the flexible polymer sheet may be considered to be “2-dimensional” given its low thickness. A flexible polymer sheet may be manipulated, such as folded or rolled to form a filled solid shape. Such as, folded to form a cuboid structure, or rolled to form a solid (filled) cylinder. Seeding cells on to a 2D porous scaffold (or flexible polymer sheet) may be more controllable than seeding onto a 3-dimensional (i.e. a filled solid shape having a lower length to thickness ratio and a lower width to thickness ratio than a sheet) porous scaffold. In addition, the use of a flexible polymer sheet may help to circumvent problems of seeding a larger 3D flexible polymer. For example, the use of a sheet that is manipulated to provide a solid 3D structure in use may allow for enhanced rapidness and scalability when producing the constructs described herein. In addition the use of a sheet that is manipulated to provide a solid 3D structure in use may help improve efficiency, uniformity and/or speed of cell seeding.

The dimensions of the construct may be selected depending on the final desired product and/or in respect of the bioreactor system the construct may be used with. The length and width of the construct may be selected in order to provide specific dimensions when the construct is manipulated to a 3-D filled shape. For example, the final dimensions of the 3-D structure made from a polymeric sheet having a thickness of 0.5 may be calculated using the formula:

$$L = 2 π [ 1.25 ( D - 2 ) + 0.5 ( \frac{( D - 2 ) ( D - 3 )}{2} ) ]$$

Where D is the cylindrical diameter and L is the required length to provide a cylinder having a diameter equal to D.

For example if a cylinder with the diameter of D is required, then a rectangular stripe of the polymeric sheet having a thickness of 0.5 mm with length of L would be required. The height of the cylinder is equal to the width of the rectangular strip. For example, if a cylinder with diameter of 13 mm and height of 40 mm, a rectangular flat scaffold of 259 mm long and 40 mm wide would be required (to be rolled). If the polymeric sheet is thinner, a longer L would be required (L′). In those cases the length of the polymeric sheet can be calculated by the formula:

$$L ’ = L \times ( 0.5 / ∅ )$$

where ø is the thickness of the sheet.

When the construct is manipulated, for example rolled, the 3-D shape produced may have a cross sectional length of at least 6 mm and a width equal to the width of the construct prior to being manipulated.

A 3D-filled shape may also formed by folding the polymeric sheet. In such a case the width of the polymeric sheet is equal to the width of the manipulated construct. The final thickness of the manipulated construct will be determined by the thickness of the sheet and the number of folds. The polymeric sheets may also be stacked on top of each other in order to form a 3D-filled shape.

The construct includes channels extending lengthwise across a surface or within the flexible polymer sheet. The channels are separate and distinct to any pores of the flexible polymer sheet (for example when a porous polymer is used). The term channel refers to a passage or duct through which a liquid or gas can flow through. The channels extend lengthwise across or within the flexible polymer sheet. That is to say that the channels form a defined void in the flexible polymer sheet extending from one side of the flexible polymer sheet to another side of the flexible polymer sheet in a direction across the flexible polymer sheet. That is to say the channels extend in a direction of the greatest length of the flexible polymer sheet and not through the thickness of the flexible polymer sheet. In some examples as described below, the channels may be closed channels and therefore the channels may extend within the sheet. The channels may extend from one surface of the flexible polymer sheet to an opposing surface of the flexible polymer sheet. For example, the channels may extend between one thin end surface of the flexible polymer sheet to the opposing thin end surface, either within the flexible polymer sheet or across at least one surface of the flexible polymer sheet. As such, the channels may each provide an opening in one surface of the flexible polymer sheet membrane that extends through the body of the flexible polymer sheet to an opposing surface of the flexible polymer sheet. Thus, providing a flexible polymer sheet with a number of apertures that extend across at least one surface of the flexible polymer sheet or within the flexible polymer sheet.

The channels may be aligned with each other. For example, each channel may be aligned with each other channel. The channels may be parallelly aligned. Such as, all channels extending in the same direction through the porous scaffold membrane. The channels may not be aligned. The channels may only be substantially aligned. For example, substantially parallelly aligned. Aligned and/or substantially aligned channels may allow for consistent flow of liquids (such as cell culture media) through all the channels. This may allow for all cells to receive a constant amount of culture media when cultured on the construct. This may allow for consistent growth and properties of cells grown on a construct.

The channels may be of any shape. For example, the channels may have a circular cross-section (i.e. are cylindrical), semi-circular cross-section, triangular cross-section (i.e. a pyramidal), cuboid cross-section (e.g. square or rectangular cross-section), trapezoidal or any polygonal cross-section. The channels may each have the same shape or each channel may have a different shape.

The channels may be open or closed channels. Open channels refers to a channel that is not enclosed, for example in the case of a square shaped channel the channel only has three surfaces or for example an open channel may have a “U” or “V” shape. In one example, when the flexible polymer sheet is non-porous or as pores having an average pore diameter between 0 to 49 μm the channels may be open channels.

Open channels may have an average width along the length of the channel from 20 μm to 700 μm. The open channels may have an average channel width from 50 μm to 500 μm. The open channels may have an average channel width from 100 μm to 500 μm. The open channels may have an average channel width from 150 μm to 300 μm. The open channels may have an average channel width from 190 μm to 210 μm. In one example the open channels may have an average width of 200 μm. For example, an average width of 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm, 195 μm, 200 μm, 205 μm, 210 μm, 215 μm, 220 μm, 225 μm, 230 μm, 235 μm, 240 μm, 245 μm, 250 μm, 255 μm, 260 μm, 265 μm, 270 μm, 275 μm, 280 μm, 285 μm, 290 μm, 295 μm, 300 μm, 305 μm, 310 μm, 315 μm, 320 μm, 325 μm, 330 μm, 335 μm, 340 μm, 345 μm, 350 μm, 355 μm, 360 μm, 365 μm, 370 μm, 375 μm, 380 μm, 385 μm, 390 μm, 395 μm, 400 μm, 405 μm, 410 μm, 415 μm, 420 μm, 425 μm, 430 μm, 435 μm, 440 μm, 445 μm, 450 μm, 455 μm, 460 μm, 465 μm, 470 μm, 475 μm, 480 μm, 485 μm, 490 μm, 495 μm, 500 μm, 505 μm, 510 μm, 515 μm, 520 μm, 525 μm, 530 μm, 535 μm, 540 μm, 545 μm, 550 μm, 555 μm, 560 μm, 565 μm, 570 μm, 575 μm, 580 μm, 585 μm, 590 μm, 595 μm, 600 μm, 605 μm, 610 μm, 615 μm, 620 μm, 625 μm, 630 μm, 635 μm, 640 μm, 645 μm, 650 μm, 655 μm, 660 μm, 665 μm, 670 μm, 675 μm, 680 μm, 685 μm, 690 μm, 695 μm, or 700 μm. In the case of a semi-circular shaped channel, the width may be a diameter. In some cases, the width is the width measured at the widest point of the open channel.

Open channels may have an average depth from 50 to 500 μm. Open channels may have an average depth from 100 to 500 μm. For example and average depth of 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm, 140 μm, 145 μm, 150 μm, 155 μm, 160 μm, 165 μm, 170 μm, 175 μm, 180 μm, 185 μm, 190 μm, 195 μm, 200 μm, 205 μm, 210 μm, 215 μm, 220 μm, 225 μm, 230 μm, 235 μm, 240 μm, 245 μm, 250 μm, 255 μm, 260 μm, 265 μm, 270 μm, 275 μm, 280 μm, 285 μm, 290 μm, 295 μm, 300 μm, 305 μm, 310 μm, 315 μm, 320 μm, 325 μm, 330 μm, 335 μm, 340 μm, 345 μm, 350 μm, 355 μm, 360 μm, 365 μm, 370 μm, 375 μm, 380 μm, 385 μm, 390 μm, 395 μm, 400 μm, 405 μm, 410 μm, 415 μm, 420 μm, 425 μm, 430 μm, 435 μm, 440 μm, 445 μm, 450 μm, 455 μm, 460 μm, 465 μm, 470 μm, 475 μm, 480 μm, 485 μm, 490 μm, 495 μm or 500 μm.

The open channels may have cells seeded into the channels. For examples, when in use cells may be disposed on at least a lower surface (or bottom) of an open channel. The cells may then be cultured within the open channel. The width of the channel may be dependent on the size of the cells being cultured. The open channels may be considered as suitable for hosting cells therein. In use, the open channels also act as a conduit for the transport of culture media to cells within the channels. Thus, the open channels are for hosting cells and transporting culture medium to the cells. Cells may be cultured, for example expanded, proliferated and/or differentiated within the channels when the construct is in use.

The open channels may be formed by etching a surface at least one flexible polymer sheet. The channels may be etched as grooves onto a surface of the flexible polymer sheet.

In other examples, slits may be etched though the surface of the flexible polymer sheet. That is to say that the surface is etched with slits or lengthwise openings that extended through the thickness of the flexible polymer sheet. In such examples, the channels are formed by creating a laminate structure by binding a first flexible polymer sheet including the slits to a second polymer sheet. The second polymer sheet therefore provides the lower surface of the channel while the upright side walls of the open channels are provided by the thickness of the of the first flexible polymer sheet.

The second flexible polymer sheet may be a flexible polymer as described herein. The second flexible polymer sheet may include the same flexible polymer as the first flexible polymer sheet or may include a different flexible polymer.

The first and second flexible polymer sheets may be bound or adhered using any suitable adhesive. In some examples, the first and second flexible polymer sheets may be adhered by a flexible polymer as described herein.

When the flexible polymer sheet comprises a non-porous polymer and/or open channels as described herein, the flexible polymer sheet may have an average thickness between 30 μm to 1000 μm. In the case of multiple flexible polymer sheets, such as a laminate structure as described, the construct may have a thickness between 30 μm to 1000 μm. For example, the at least one flexible polymer sheet may have an average thickness of 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, 70 μm, 71 μm, 72 μm, 73 μm, 74 μm, 75 μm, 76 μm, 77 μm, 78 μm, 79 μm, 80 μm, 81 μm, 82 μm, 83 μm, 84 μm, 85 μm, 86 μm, 87 μm, 88 μm, 89 μm, 90 μm, 91 μm, 92 μm, 93 μm, 94 μm, 95 μm, 96 μm, 97 μm, 98 μm, 99 μm, 100 μm, 105 μm, 110 μm, 115 μm, 120 μm, 125 μm, 130 μm, 135 μm, 140 μm, 145 μm, 150 μm, 155 μm, 160 μm, 165 μm, 170 μm, 175 μm, 180 μm, 185 μm, 190 μm, 195 μm, 200 μm, 205 μm, 210 μm, 215 μm, 220 μm, 225 μm, 230 μm, 235 μm, 240 μm, 245 μm, 250 μm, 255 μm, 260 μm, 265 μm, 270 μm, 275 μm, 280 μm, 285 μm, 290 μm, 295 μm, 300 μm, 305 μm, 310 μm, 315 μm, 320 μm, 325 μm, 330 μm, 335 μm, 340 μm, 345 μm, 350 μm, 355 μm, 360 μm, 365 μm, 370 μm, 375 μm, 380 μm, 385 μm, 390 μm, 395 μm, 400 μm, 405 μm, 410 μm, 415 μm, 420 μm, 425 μm, 430 μm, 435 μm, 440 μm, 445 μm, 450 μm, 455 μm, 460 μm, 465 μm, 470 μm, 475 μm, 480 μm, 485 μm, 490 μm, 495 μm, 500 μm, 505 μm, 510 μm, 515 μm, 520 μm, 525 μm, 530 μm, 535 μm, 540 μm, 545 μm, 550 μm, 555 μm, 560 μm, 565 μm, 570 μm, 575 μm, 580 μm, 585 μm, 590 μm, 595 μm, 600 μm, 605 μm, 610 μm, 615 μm, 620 μm, 625 μm, 630 μm, 635 μm, 640 μm, 645 μm, 650 μm, 655 μm, 660 μm, 665 μm, 670 μm, 675 μm, 680 μm, 685 μm, 690 μm, 695 μm, 700 μm, 705 μm, 710 μm, 715 μm, 720 μm, 725 μm, 730 μm, 735 μm, 740 μm, 745 μm, 750 μm, 755 μm, 760 μm, 765 μm, 770 μm, 775 μm, 780 μm, 785 μm, 790 μm, 795 μm, 800 μm, 805 μm, 810 μm, 815 μm, 820 μm, 825 μm, 830 μm, 835 μm, 840 μm, 845 μm, 850 μm, 855 μm, 860 μm, 865 μm, 870 μm, 875 μm, 880 μm, 885 μm, 890 μm, 895 μm, 900 μm, 905 μm, 910 μm, 915 μm, 920 μm, 925 μm, 930 μm, 935 μm, 940 μm, 945 μm, 950 μm, 955 μm, 960 μm, 965 μm, 970 μm, 975 μm, 980 μm, 985 μm, 990 μm, 995 μm, 1000 μm or any value falling between the average thicknesses listed.

When the flexible polymer sheet comprises a porous polymer as described herein (i.e. is a porous flexible polymer sheet) the channels may be closed channels. A closed channel refers to a channel, which is completely closed apart from an inlet and/or an outlet, i.e. it is tubular, that is, the channel is limited everywhere by walls perpendicular to its main flow direction. The closed channels may have an average width between 5 μm to 1000 μm. The closed channels may have an average width between 50 μm to 1000 μm. The closed channels may have an average width between 70 μm to 700 μm. For example, the closed channels may have an average width of 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm, 140 μm, 145 μm, 150 μm, 155 μm, 160 μm, 165 μm, 170 μm, 175 μm, 180 μm, 185 μm, 190 μm, 195 μm, 200 μm, 205 μm, 210 μm, 215 μm, 220 μm, 225 μm, 230 μm, 235 μm, 240 μm, 245 μm, 250 μm, 255 μm, 260 μm, 265 μm, 270 μm, 275 μm, 280 μm, 285 μm, 290 μm, 295 μm, 300 μm, 305 μm, 310 μm, 315 μm, 320 μm, 325 μm, 330 μm, 335 μm, 340 μm, 345 μm, 350 μm, 355 μm, 360 μm, 365 μm, 370 μm, 375 μm, 380 μm, 385 μm, 390 μm, 395 μm, 400 μm, 405 μm, 410 μm, 415 μm, 420 μm, 425 μm, 430 μm, 435 μm, 440 μm, 445 μm, 450 μm, 455 μm, 460 μm, 465 μm, 470 μm, 475 μm, 480 μm, 485 μm, 490 μm, 495 μm, 500 μm, 505 μm, 510 μm, 515 μm, 520 μm, 525 μm, 530 μm, 535 μm, 540 μm, 545 μm, 550 μm, 555 μm, 560 μm, 565 μm, 570 μm, 575 μm, 580 μm, 585 μm, 590 μm, 595 μm, 600 μm, 605 μm, 610 μm, 615 μm, 620 μm, 625 μm, 630 μm, 635 μm, 640 μm, 645 μm, 650 μm, 655 μm, 660 μm, 665 μm, 670 μm, 675 μm, 680 μm, 685 μm, 690 μm, 695 μm, 700 μm, 705 μm, 710 μm, 715 μm, 720 μm, 725 μm, 730 μm, 735 μm, 740 μm, 745 μm, 750 μm, 755 μm, 760 μm, 765 μm, 770 μm, 775 μm, 780 μm, 785 μm, 790 μm, 795 μm, 800 μm, 805 μm, 810 μm, 815 μm, 820 μm, 825 μm, 830 μm, 835 μm, 840 μm, 845 μm, 850 μm, 855 μm, 860 μm, 865 μm, 870 μm, 875 μm, 880 μm, 885 μm, 890 μm, 895 μm, 900 μm, 905 μm, 910 μm, 915 μm, 920 μm, 925 μm, 930 μm, 935 μm, 940 μm, 945 μm, 950 μm, 955 μm, 960 μm, 965 μm, 970 μm, 975 μm, 980 μm, 985 μm, 990 μm, 995 μm, 1000 μm or any value falling between the average widths listed. As the closed channels may be tubular the width may be an average diameter. The closed channels have walls made from the flexible porous polymer sheet and as such may be considered to be porous channels. The pores of the closed channels are the same as the pores of the flexible porous polymer sheet as described herein.

A construct including a flexible porous polymer sheet and closed channels may have an average thickness of between 100 μm to 1000 μm. For example, the average thickness may be 100 μm, 105 μm, 110 μm, 115 μm, 120 μm, 125 μm, 130 μm, 135 μm, 140 μm, 145 μm, 150 μm, 155 μm, 160 μm, 165 μm, 170 μm, 175 μm, 180 μm, 185 μm, 190 μm, 195 μm, 200 μm, 205 μm, 210 μm, 215 μm, 220 μm, 225 μm, 230 μm, 235 μm, 240 μm, 245 μm, 250 μm, 255 μm, 260 μm, 265 μm, 270 μm, 275 μm, 280 μm, 285 μm, 290 μm, 295 μm, 300 μm, 305 μm, 310 μm, 315 μm, 320 μm, 325 μm, 330 μm, 335 μm, 340 μm, 345 μm, 350 μm, 355 μm, 360 μm, 365 μm, 370 μm, 375 μm, 380 μm, 385 μm, 390 μm, 395 μm, 400 μm, 405 μm, 410 μm, 415 μm, 420 μm, 425 μm, 430 μm, 435 μm, 440 μm, 445 μm, 450 μm, 455 μm, 460 μm, 465 μm, 470 μm, 475 μm, 480 μm, 485 μm, 490 μm, 495 μm, 500 μm, 505 μm, 510 μm, 515 μm, 520 μm, 525 μm, 530 μm, 535 μm, 540 μm, 545 μm, 550 μm, 555 μm, 560 μm, 565 μm, 570 μm, 575 μm, 580 μm, 585 μm, 590 μm, 595 μm, 600 μm, 605 μm, 610 μm, 615 μm, 620 μm, 625 μm, 630 μm, 635 μm, 640 μm, 645 μm, 650 μm, 655 μm, 660 μm, 665 μm, 670 μm, 675 μm, 680 μm, 685 μm, 690 μm, 695 μm, 700 μm, 705 μm, 710 μm, 715 μm, 720 μm, 725 μm, 730 μm, 735 μm, 740 μm, 745 μm, 750 μm, 755 μm, 760 μm, 765 μm, 770 μm, 775 μm, 780 μm, 785 μm, 790 μm, 795 μm, 800 μm, 805 μm, 810 μm, 815 μm, 820 μm, 825 μm, 830 μm, 835 μm, 840 μm, 845 μm, 850 μm, 855 μm, 860 μm, 865 μm, 870 μm, 875 μm, 880 μm, 885 μm, 890 μm, 895 μm, 900 μm, 905 μm, 910 μm, 915 μm, 920 μm, 925 μm, 930 μm, 935 μm, 940 μm, 945 μm, 950 μm, 955 μm, 960 μm, 965 μm, 970 μm, 975 μm, 980 μm, 985 μm, 990 μm, 995 μm, 1000 μm or any value falling between the average thicknesses listed.

The closed channels may be hollow channels. That is the closed channels have no material within the closed channels when not in use. The closed channels may be provided with a channel forming member therein. For example, the closed channels may include a wire, rod or other suitable channel forming member therein.

When a porous polymer as described herein is used to form the flexible polymer sheet, the pores of the porous polymer may host and/or maintain cells therein. As such, cells may be cultured within the pores of the porous polymer. The channels act as conduits to transport cell culture medium to the cells within the pores as well as transporting waste products from the cells located within the pores of the flexible porous polymer sheet.

The open or closed channels may be spaced apart by any suitable distance. For example, each channel may be spaced from the next adjacent channel by an average distance between 100 μm to 1500 μm. For example the channels may be spaced by an average distance of 100 μm, 105 μm, 110 μm, 115 μm, 120 μm, 125 μm, 130 μm, 135 μm, 140 μm, 145 μm, 150 μm, 155 μm, 160 μm, 165 μm, 170 μm, 175 μm, 180 μm, 185 μm, 190 μm, 195 μm, 200 μm, 205 μm, 210 μm, 215 μm, 220 μm, 225 μm, 230 μm, 235 μm, 240 μm, 245 μm, 250 μm, 255 μm, 260 μm, 265 μm, 270 μm, 275 μm, 280 μm, 285 μm, 290 μm, 295 μm, 300 μm, 305 μm, 310 μm, 315 μm, 320 μm, 325 μm, 330 μm, 335 μm, 340 μm, 345 μm, 350 μm, 355 μm, 360 μm, 365 μm, 370 μm, 375 μm, 380 μm, 385 μm, 390 μm, 395 μm, 400 μm, 405 μm, 410 μm, 415 μm, 420 μm, 425 μm, 430 μm, 435 μm, 440 μm, 445 μm, 450 μm, 455 μm, 460 μm, 465 μm, 470 μm, 475 μm, 480 μm, 485 μm, 490 μm, 495 μm, 500 μm, 505 μm, 510 μm, 515 μm, 520 μm, 525 μm, 530 μm, 535 μm, 540 μm, 545 μm, 550 μm, 555 μm, 560 μm, 565 μm, 570 μm, 575 μm, 580 μm, 585 μm, 590 μm, 595 μm, 600 μm, 605 μm, 610 μm, 615 μm, 620 μm, 625 μm, 630 μm, 635 μm, 640 μm, 645 μm, 650 μm, 655 μm, 660 μm, 665 μm, 670 μm, 675 μm, 680 μm, 685 μm, 690 μm, 695 μm, 700 μm, 705 μm, 710 μm, 715 μm, 720 μm, 725 μm, 730 μm, 735 μm, 740 μm, 745 μm, 750 μm, 755 μm, 760 μm, 765 μm, 770 μm, 775 μm, 780 μm, 785 μm, 790 μm, 795 μm, 800 μm, 805 μm, 810 μm, 815 μm, 820 μm, 825 μm, 830 μm, 835 μm, 840 μm, 845 μm, 850 μm, 855 μm, 860 μm, 865 μm, 870 μm, 875 μm, 880 μm, 885 μm, 890 μm, 895 μm, 900 μm, 905 μm, 910 μm, 915 μm, 920 μm, 925 μm, 930 μm, 935 μm, 940 μm, 945 μm, 950 μm, 955 μm, 960 μm, 965 μm, 970 μm, 975 μm, 980 μm, 985 μm, 990 μm, 995 μm, 1000 μm, 1005 μm, 1010 μm, 1015 μm, 1020 μm, 1025 μm, 1030 μm, 1035 μm, 1040 μm, 1045 μm, 1050 μm, 1055 μm, 1060 μm, 1065 μm, 1070 μm, 1075 μm, 1080 μm, 1085 μm, 1090 μm, 1095 μm, 1100 μm, 1105 μm, 1110 μm, 1115 μm, 1120 μm, 1125 μm, 1130 μm, 1135 μm, 1140 μm, 1145 μm, 1150 μm, 1155 μm, 1160 μm, 1165 μm, 1170 μm, 1175 μm, 1180 μm, 1185 μm, 1190 μm, 1195 μm, 1200 μm, 1205 μm, 1210 μm, 1215 μm, 1220 μm, 1225 μm, 1230 μm, 1235 μm, 1240 μm, 1245 μm, 1250 μm, 1255 μm, 1260 μm, 1265 μm, 1270 μm, 1275 μm, 1280 μm, 1285 μm, 1290 μm, 1295 μm, 1300 μm, 1305 μm, 1310 μm, 1315 μm, 1320 μm, 1325 μm, 1330 μm, 1335 μm, 1340 μm, 1345 μm, 1350 μm, 1355 μm, 1360 μm, 1365 μm, 1370 μm, 1375 μm, 1380 μm, 1385 μm, 1390 μm, 1395 μm, 1400 μm, 1405 μm, 1410 μm, 1415 μm, 1420 μm, 1425 μm, 1430 μm, 1435 μm, 1440 μm, 1445 μm, 1450 μm, 1455 μm, 1460 μm, 1465 μm, 1470 μm, 1475 μm, 1480 μm, 1485 μm, 1490 μm, 1495 μm, 1500 μm or any value falling between the average distances listed.

As the channels may be aligned, the average distance between the channels may be maintained for the entire length of the channels. That is to say that the average distance between adjacent channels is the same and does not alter along the entire length of each channel. The channels may each have a uniform cross-section. For example, all the channels have the same shape and size and along the length of the channel and each channel is uniform in respect of each other channel.

The number of channels is relative to the length of the polymeric sheet. The number of channels may be provided as the number of channels per length unit of the flexible polymer sheet. For example a flexible porous polymer sheet may have at least 0.4 channels per mm. For example non-porous flexible polymer sheet may have at least 0.7 channels per mm The number of channels may be selected based on the dimensions of the channels and the spacing between the channels. One consideration for selecting the number of channels is the oxygen transfer threshold or oxygen transfer rate. The oxygen transfer rate refers to the amount of oxygen that passes through a substance or in this case a scaffold over a period of time. If the flexible polymer sheet (whether porous or non-porous) has a relatively low oxygen transfer rate then the number of channels may be increased proportionally to the oxygen transfer rate. For example if x=the oxygen transfer threshold (in μm) then the spacing between each channel, for example between two adjacent channels, may be at most 2× (μm). This may provide a construct that allows for transfer of oxygen that is suitable for maintaining cells. If the channel width is y, then the number of channels (N) per mm may be calculated by the equation:

$$N = \frac{1000}{2 x + y}$$

Providing uniform channels (e.g. all channels having the same shape, spacing distance and/or size that are substantially aligned) may allow for enhanced rapidness and scalability when producing the constructs described herein. In addition uniform channels may help improve efficiency, uniformity and/or speed of cell seeding. Uniform channels may also help to stimulate cell differentiation. Cells that enter a differentiation phase may lead to higher levels of protein being produced leading to a more nutritious comestible product being produced by the constructs.

Flexible polymer sheet refers to a sheet of polymer material or polymer substrate that can be deformed and manipulated, such as rolled and/or folded without the sheet being damaged, ruptured and/or broken. That is to say that a flexible polymer sheet can be rolled and unrolled without the sheet tearing, splitting or rupturing. As the channels are formed using a flexible polymer sheet, when the construct is manipulated the channels are maintained. That is to say that the channels are not crushed or closed due to manipulation. Manipulation may lead to a change in the shape or size of the channels but does not alter the utility of the channels for hosting, culturing and/or maintaining cells and/or transporting cell culture medium to the cells.

The flexibility of construct allows for the construct to be rolled into a solid 3D structure as described herein. Thus the use of a flexible polymer sheet allows for the construct to be seeded in 2D and is then manipulated to provide a 3D cell culture, thus negating the complexity and any disadvantages associated with seeding 3D structures.

The flexibility of materials such as the flexible polymer sheets described herein may be determined by the elastic or Young's modulus of the material. A number of methods are known for measuring flexibility of materials and determining the Young's modulus. Elastic modulus can be calculated by dividing the stress by the strain and it is a property that is dependent on the type of material and not on the size and shape. Elastic or Young's modulus by force displacement measurement methods. Young's modulus measures the resistance of a material to elastic (recoverable) deformation under load. A stiff material has a high Young's modulus and changes its shape only slightly under elastic loads (e.g. diamond). A flexible material has a low Young's modulus and changes its shape considerably (e.g. rubbers). Generally, flexible materials have a Young's modulus of less than about 1 GPa (or 1000 MPa). As such, flexible polymers of the invention may have a Youngs modulus of less than 1000 MPa. In some examples, the flexible polymers of the invention may have a Young's modulus of less than 800 MPa. In some examples, the flexible polymers of the invention may have a Young's modulus of less than 700 MPa. In some examples, the flexible polymers of the invention may have a Young's modulus of less than 600 MPa. In some examples, the flexible polymers of the invention may have a Young's modulus of less than 500 MPa. In some examples, the flexible polymers of the invention may have a Young's modulus of less than 200 MPa. In some examples, the flexible polymers of the invention may have a Young's modulus of less than 100 MPa.

Flexible refers to a material that is capable of undergoing strain, such as bending or stretching (such as when folded or rolled), without adverse impact of physical characteristics, such as irreversible break-down associated with material fracture, for example. “Stretchable” is used in a similar manner to refer to reversible strain without material fracture.

In some examples, the flexible polymer is a non-porous flexible polymer as described herein and may have a Young's modulus of less than 500 MPa. For example, less than 500, 400, 300, 200, 100, 50, 40, 30, or 20 MPa. For example, non-porous flexible polymers may have a Young's modulus of from 500 MPa to 100 MPa. For example, from about 250 MPa to 300 MPa. For example, about 280 MPa.

In some examples, the flexible polymer is a porous flexible polymer as described herein and may have a Young's modulus of less than 150 MPa. For example, less than 150, 140, 130, 120, 110, 100, 90, 80, 70, 60, 50, 40, 30, 20 or 10 MPa. For example, porous flexible polymers may have a Young's modulus of from 150 MPa to 50 MPa. For example, about 100 to 120 MPa. For example, about 110 MPa.

In some examples, the flexible polymer is dry BCS and has a Young's modulus of less than 800 MPa. For example, less than 800, 700, 600, 500, 400, 300, 200, 100, 50, 40, 30, or 20 MPa. For example, the flexible polymer may have a Young's modulus of from 500 MPa to 800 MPa. For example, a Young's modulus of from about 600 MPa to about 800 MPa. For example, the BCS may have a Young's modulus from 700 to 760 MPa.

In some examples, the flexible polymer is wet BCS and has a Young's modulus of less than 100 MPa. For example, less than 100, 95, 90, 85, 80, 75, 70, 65, 60, 55, 50, 45, 40, 35, 30, 25, 20, 15, 10 or 5 MPa. For example, the flexible polymer may have a Young's modulus of from 10 MPa to 50 MPa. For example, a Young's modulus of from about 20 MPa to about 45 MPa. For example, from about 25 MPa to about 40 MPa. For example, the BCS may have a Young's modulus of about 38 MPa.

The flexible polymers of the invention may be considered at least partially elastic or elastically deformable. That is to say that after being deformed the flexible polymers may return to their original shape and/or size. Elastic deformation may be determined by measuring using similar methods as those for determining the Young's modulus. The elastic region or area of a material may be expressed as a percentage of total length of the material sample being tested.

For example, the flexible polymers of the invention may have an elastic area of more than 1% of the total length of the flexible polymer sheet. In some examples, the flexible polymers of the invention may have an elastic area from 1% of the total length of the flexible polymer sheet up to 99% of the total length of the flexible polymer sheet. For example, from 1% to about 20%. For example, from 1% to about 15%.

In some examples, the flexible polymer is a non-porous flexible polymer as described herein and may have an elastic area of between 1 and 10% of the total length of the flexible polymer sheet. For example, non-porous flexible polymers may have an elastic area of about 5% of the total length of the polymer sheet. For example about, 5% of the total length of the polymer sheet.

In some examples, the flexible polymer is a porous flexible polymer as described herein and may have an elastic area of between 1 and 3% of the total length of the flexible polymer sheet. For examples, from about 1.4% to about 2% of the total length of the polymer sheet.

In some examples, the flexible polymer is wet BCS and have an elastic area of between 1 and 15% of the total length of the flexible polymer sheet. For example, an elastic area of between 5 and 12% of the total length of the flexible polymer sheet. For example, an elastic area of between 7 and 10% of the total length of the flexible polymer sheet.

The constructs described herein may be made by made using different methods depending the porosity of the polymer used for the flexible polymer sheet.

When the flexible polymer sheet comprises a porous polymer as described herein the method includes dissolving a polymer in a solvent to form a mixture. The solvent may be any suitable solvent and may be selected deepening on the polymer being used. Examples of solvents include hydrocarbons, alcohols, ethers, amides and water. A combination of two or more such solvents in suitable proportions may also be used. For example, the solvent may include acetone, chloroform, dichloromethane or dimethyl formamide. The solvent may include acetone. The amount of polymer dissolved in solvent may be determined based on the polymer being used. In some examples the polymer may be dissolved in an amount of at least 1% W/V of polymer to solvent. For example, PCL may be dissolved at a concentration of 8.5% W/V in the solvent.

Once the polymer has been dissolved a porogen is added to the mixture containing the dissolved polymer. The porogen may be any suitable porogen such as slat particles or PEG. The porogen may have a diameter of at most 125 μm. Salt particles having such a diameter may be prepared by any known method, such as crushing salt particles using a pestle and mortar and then sieving the crushed salt particles through a mesh that excludes any particles greater than 125 μm. The salt particles may be any suitable salt particles such as sodium chloride.

The mixture is mixed until a homogenous mixture is achieved. The mixture may be heated to help improve mixing. For example to a temperature of around 45° C.

Once the porogen has been mixed into the mixture, the mixture is applied to a planar structure. A planar structure refers to any flat substrate for example the mixture is applied to a piece of glass. The planar structure includes a plurality of channel forming members. The channel forming members are configured so as to be suspended over at least one surface of the planar structure. By not contacting the surface of the planar structure applied mixture is able to encase the channel forming members, i.e. be located above, below and between each channel forming member. The channel forming members may be wires wrapped around the planar structure or rods placed suspended over at least one surface of the planar structure.

The channel forming members may have an average diameter ranging from 5 μm to 1000 μm. The channel forming members may have an average diameter ranging from 20 μm to 1000 μm. The channel forming members may have an average diameter ranging from 70 μm to 1000 μm. For example, the channel forming members may have an average diameter of 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm, 140 μm, 145 μm, 150 μm, 155 μm, 160 μm, 165 μm, 170 μm, 175 μm, 180 μm, 185 μm, 190 μm, 195 μm, 200 μm, 205 μm, 210 μm, 215 μm, 220 μm, 225 μm, 230 μm, 235 μm, 240 μm, 245 μm, 250 μm, 255 μm, 260 μm, 265 μm, 270 μm, 275 μm, 280 μm, 285 μm, 290 μm, 295 μm, 300 μm, 305 μm, 310 μm, 315 μm, 320 μm, 325 μm, 330 μm, 335 μm, 340 μm, 345 μm, 350 μm, 355 μm, 360 μm, 365 μm, 370 μm, 375 μm, 380 μm, 385 μm, 390 μm, 395 μm, 400 μm, 405 μm, 410 μm, 415 μm, 420 μm, 425 μm, 430 μm, 435 μm, 440 μm, 445 μm, 450 μm, 455 μm, 460 μm, 465 μm, 470 μm, 475 μm, 480 μm, 485 μm, 490 μm, 495 μm, 500 μm, 505 μm, 510 μm, 515 μm, 520 μm, 525 μm, 530 μm, 535 μm, 540 μm, 545 μm, 550 μm, 555 μm, 560 μm, 565 μm, 570 μm, 575 μm, 580 μm, 585 μm, 590 μm, 595 μm, 600 μm, 605 μm, 610 μm, 615 μm, 620 μm, 625 μm, 630 μm, 635 μm, 640 μm, 645 μm, 650 μm, 655 μm, 660 μm, 665 μm, 670 μm, 675 μm, 680 μm, 685 μm, 690 μm, 695 μm, 700 μm, 705 μm, 710 μm, 715 μm, 720 μm, 725 μm, 730 μm, 735 μm, 740 μm, 745 μm, 750 μm, 755 μm, 760 μm, 765 μm, 770 μm, 775 μm, 780 μm, 785 μm, 790 μm, 795 μm, 800 μm, 805 μm, 810 μm, 815 μm, 820 μm, 825 μm, 830 μm, 835 μm, 840 μm, 845 μm, 850 μm, 855 μm, 860 μm, 865 μm, 870 μm, 875 μm, 880 μm, 885 μm, 890 μm, 895 μm, 900 μm, 905 μm, 910 μm, 915 μm, 920 μm, 925 μm, 930 μm, 935 μm, 940 μm, 945 μm, 950 μm, 955 μm, 960 μm, 965 μm, 970 μm, 975 μm, 980 μm, 985 μm, 990 μm, 995 μm, or 1000 μm.

The mixture is disposed onto the planar structure so that the mixture encases or envelops the channel forming members. The planar structure with the applied mixture is then immersed in an anti-solvent solution. The anti-solvent may be selected depending on the solvent used. For example, for an acetone containing solvent the anti-solvent may be water. The planar structure with the mixture applied may be immersed in the anti-solvent for at least 1 hour. For example, at least 1 hour, 2 hours, 3 hours, 4 hours, 5 hours, 6, hours, 7 hours, 8 hours, 9 hours, 10 hours, 12 hours, 14 hours, 16 hours, 18 hours, 20 hours, 22 hours, 24 hours or more.

The anti-solvent extracts the solvent from the applied polymer and leaves a flexible polymer sheet. Simultaneously, the anti-solvent dissolves the porogen leading to formation of a flexible porous polymer sheet with the channel forming members enclosed therein.

The planar structure with the applied mixture is then removed from the anti-solvent and dried.

The flexible polymer sheet including the channel forming members is then removed from the planar structure with the channel forming members within the flexible polymer sheet.

The channel forming members may be left within the flexible polymer sheet until the construct is seeded with cells or removed from the flexible polymer sheet. When removed, the channel forming members leave closed channels extending through the length of the flexible polymer sheet with each channel having an aperture on opposing sides of the flexible polymer sheet, such that the channels each form an inlet aperture on one surface of the flexible polymer sheet and an outlet aperture on an opposing surface of the flexible polymer sheet. The porous nature of the flexible polymer sheet provides channels with walls that include the pores of the polymer. These pores allow fluids such as culture media to contact cells seeded within the pores of the scaffold and thus allows delivery of nutrients to the cells and transport of waste products from the cells.

For forming a construct comprising a non-porous flexible polymer sheet as described herein a flexible polymer sheet may be formed by any known method, such as industrial curding, electrospinning, moulding, sheet casting, or spincoating.

The non-porous flexible polymer sheet is then etched to provide a plurality of channels on at least one surface of the flexible polymer sheet. As described above, the channels may only extend through a portion of the thickness of the flexible polymer sheet. As such the channels may be considered grooves in at least one surface of the flexible polymer sheet. In some examples described below, the channels may be etched as slits that extend through the entirety of the thickness of the flexible polymer sheet.

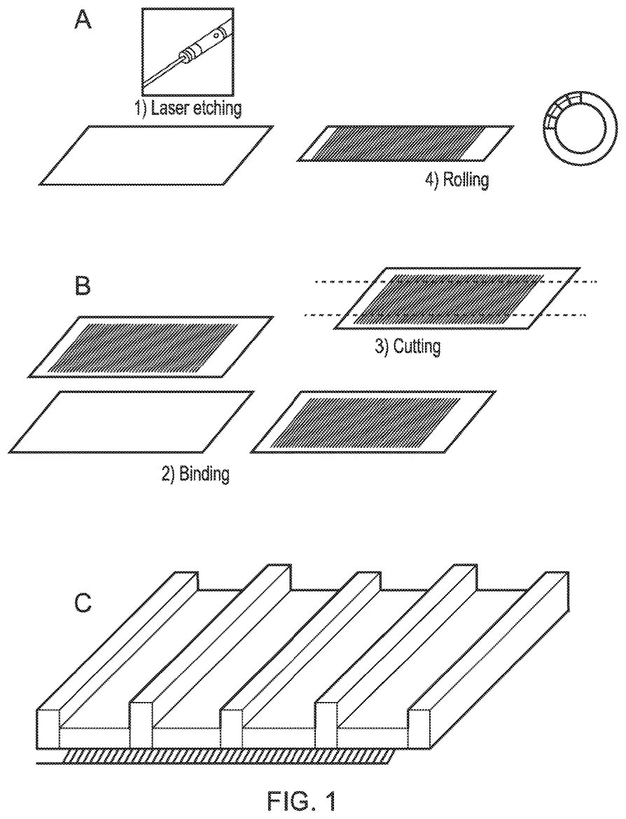

The channels (100) may extend from one surface (110) of the flexible polymer sheet to an opposing surface (120) of the flexible polymer sheet as shown in FIG. 1A. The channels (100) may be initially etched onto the at least one surface so that they do not extend to any edges of the sheet as shown in FIG. 1B. If the channels are not etched to opposing surfaces or edges of the flexible polymer sheet the flexible polymer sheet may be cut so that channels extend to opposing sides, surfaces or edges of the flexible polymer sheet as shown in FIG. 1C.

The channels may be etched using any suitable etching method such as dry etching methods such as laser etching, plasma etching, thermal etching or wet etching methods such as chemical etching methods. Dry etching methods, such as laser etching, may allow for better control of the dimensions (such as width and depth) of the channels. Etching methods may also allow for faster, more energy efficient and/or more scalable production of a construct as described herein.

When the channels are slits extending through the thickness of the flexible polymer sheet the etched flexible polymer sheet may be adhered to a second flexible polymer sheet not including any channels to form a flexible laminate structure. The second flexible polymer sheet therefore forms the base or bottom of the channels. As the channels only have three surfaces they are open channels.

The flexible polymer sheet or laminate structure may then be immersed in in a firming agent. This may help provide a construct that has reduced degradation when in use. For example, the flexible polymer sheet or laminate structure may be immersed in a food grade firming agent. Examples of food grade firming agents include calcium carbonate, calcium hydrogen sulphite, calcium citrates, calcium phosphates, calcium sulphate, calcium chloride, magnesium chloride, magnesium sulphate, calcium gluconate, or magnesium gluconate. In some examples, the firing agent is calcium chloride. The firming agent may be used at a concentration of at least 10 mM. For example, 10 mM, 20 mM, 30 mM, 40 mM, 50 mM, 60 mM, 70 mM, 80 mM, 90 mM, 100 mM or more. In some examples, the firming agent is at a concentration of 70 mM.

The flexible polymer structure or laminate structure may be immersed in the firming agent for at least 1 hour, 2 hours, 3 hours, 4 hours, 5 hours, 6, hours, 7 hours, 8 hours, 9 hours, 10 hours, 12 hours, 14 hours, 16 hours, 18 hours, 20 hours, 22 hours, 24 hours or more.

For example, if the flexible polymer sheet is formed from BCS the sheet may be immersed in a forming agent for 24 hours prior to be cut as described above.

The constructs described herein may be for use in culturing cells in a perfusion bioreactor. As such, a system for perfusion bioreactor culturing of cells including a construct as described herein is provided.

Perfusion culturing refers to a continuous culturing method in which cells are either retained in the bioreactor or fed back into it. The cell culture medium perfused through the bioreactor thus contains no cells. Perfusion based culturing methods may result in higher cell concentrations and product yields in the reactor while reducing the working volume for example in view of continuous stirred tank reactors methods and systems.

As used herein the term “perfusion bioreactor” refers to a cell culture system in which the cell culture medium (e.g., a first cell culture medium, a second cell culture medium, a culture medium, a cell proliferation cell culture medium, and/or a cell differentiation cell culture medium) is continuously replaced with fresh media. Perfusion bioreactor systems may include means (e.g., an outlet, inlet, pump, or other such device) for periodically or continuously withdrawing and adding substantially the same volume of replacement cell culture medium to the bioreactor. The addition of the replacement liquid culture can be performed substantially simultaneously with or immediately after removal of the initial cell culture medium from the bioreactor. The means for removing the liquid culture from the bioreactor and for adding the replacement liquid culture may be a single device or system. For example, the means for removing and replacing (i.e. perfusing cell culture media) may be a peristaltic pump system.

A “bioreactor” any device or system that maintains a biologically active environment for example, a chamber or vessel in which cells can be cultured. There are several different bioreactor types that differ in shape (e.g., cylindrical or otherwise), size (e.g., millilitres, litters to cubic meters), and materials (stainless steel, glass, plastic, etc.). Accordingly, the bioreactor is adapted to grow cells or tissue in cell culture. The bioreactor may be configured to receive a construct as described herein in a manipulated form. For example, the construct may be rolled to form a solid cylinder structure having cells within the channels or within the pores of a construct including a flexible porous polymer sheet.

In some examples, the construct has a solid cylindrical structure, and the bioreactor includes a cylindrical chamber for receiving the manipulated cylindrical structure therein. The chamber may have an internal width or diameter that is substantially the same as a width of the manipulated construct or vice versa (i.e. the manipulated construct has a width or diameter substantially equal to the internal width of the chamber). The volume of chamber may be substantially equal to the volume of a manipulated construct or vice versa (i.e. the volume of a manipulated construct may be substantially equal to the internal volume of the chamber). That is to say that the manipulated construct may substantially fill the chamber.

The constructs provided herein may be used for methods of culturing cells using a perfusion bioreactor system as described. Prior to using the constructs for methods of culturing cells may include sterilising the construct. For example, by applying a sterilization solution such as a solution comprising ethanol or acetone. Other suitable sterilization methods and solutions will be known. The construct may also be washed prior to use. For example, the constructs may be washed with a buffer, such as phosphate buffered saline. The construct may be washed or further washed with a culture medium as described herein not including cells.

The methods include applying a cell support agent to a construct. The cell support agent may provide an environment which allows cells to attach, replicate, differentiate and/or migrate. This cell support agent may also protect the cells from the sheer stresses of the perfusing flow when the perfusion bioreactor is in use. Suitable cell support agents may be agent that can provide a hydrogel that provides a suitable environment for cell maintenance.

The cell support agent may be fibrinogen. Fibrinogen includes natural fibrinogen, recombinant fibrinogen or a fibrinogen derivative that can be converted to fibrin using thrombin (for example, a natural or recombinant fibrin monomer or a fibrin monomer derivative). Fibrinogen can be obtained from any source and from any species such as bovine fibrin.

The cells may be seeded onto a construct having fibrinogen applied in a cell seeding solution including thrombin. The inclusion of the thrombin in the composition with the cells may lead to polymerization of fibrinogen to fibrin. Fibrin can be highly adhesive, have biomechanical stiffness, be biocompatible, and be degradable.

The cell support agent may be gelatine. The gelatine may be animal and/or plant gelatine such as bovine gelatine, fish gelatine, algae gelatine, and mixtures thereof. Gelatine may be a heterogeneous mixture of water-soluble proteins of high average molecular weight derived from the collagen-containing parts of animals, such as skin, bone and ossein by hydrolytic action, usually either acid hydrolysis or alkaline hydrolysis.

The cell support agent may be applied in any suitable manner, such as pipetting the cell support agent onto a construct. The cell support agent may be applied to the construct at any suitable concentration and/or amount. The concentration and/or amount of cell support agent may be selected based on the dimensions of the construct. The cell support agent may be applied at a concentration of at least 5 mg/ml. For example the cell support agent may be applied at a concentration of at least 5, 10, 15, 20, 25, 30, 35, 40, 45, 50, 55, 60, 70, 75 or 80 mg/ml. For example, fibrinogen may be applied to the construct at a concentration of 20 mg/ml.

Once the cell support agent has been applied to the construct, cells are seeded onto the construct. Cells to be seeded may be in a cell seeding solution. The cell seeding solution may be a cell culture medium as described herein. Prior to seeding the construct, the cells may be cultured in the cell seeding solution (i.e. pre-cultured). Cells may be pre-cultured to a desired to a desired density prior to seeding. For example, cell may be pre-cultured to a density from 3500 cells/cm² to 100,000 cells/cm². In some examples cells may be pre-cultured to a density greater than 100,000 cells/cm². For example, cell may be pre-cultured to a density of at least 3500 cells/cm². For example, cells may be pre-cultured to a density 3500 cells/cm², 4000 cells/cm², 4500 cells/cm², 5000 cells/cm², 5500 cells/cm², 6000 cells/cm², 6500 cells/cm², 7000 cells/cm², 7500 cells/cm², 8000 cells/cm², 8500 cells/cm², 9000 cells/cm². Cells may be pre-cultured to a density of 5000 cells/cm².

Cells may be pre-cultured to a suitable confluence. For example, cells may be pre-cultured to a confluence of at least 50%, 55%, 60%, 65%, 70%, 75%, 80%, 85%, 90%, or 95%. In some examples, cell may be pre-cultured to a confluence of 80%.

As mentioned above the cell seeding solution may include additional agents such as crosslinking agents (e.g. thrombin). For example, thrombin may be included at a concentration of at least 0.1 U/ml. For example, at least 0.1 U/ml, 0.2 U/ml, 0.3 U/ml, 0.4 U/ml, 0.5 U/ml, 0.6 U/ml, 0.7 U/ml, 0.8 U/ml, 0.9 U/ml, 1 U/ml, 2 U/ml, 3 U/ml, 4 U/ml, 5 U/ml, 6 U/ml, 7 U/ml, 8 U/ml, 9 U/ml, 10 U/ml, 20 U/ml, 30 U/ml, 40 U/ml, 50 U/ml, 60 U/ml, 70 U/ml, 80 U/ml, 90 U/ml, or 100 U/ml. In some examples, thrombin may be included in the cell seeding solution at a concentration of 4 U/ml.

In some examples, such as when the cell support agent is gelatin the cell seeding solution may include 90% V/V ethanol/water solution containing 25 mM EDC (N-ethyl-N′-(3-(dimethylamino) propyl) carbodiimide (EDC) and 10 mM N-hydroxysuccinimide (NHS).

Pre-cultured cells may then be applied to the construct having the cell support agent thereon. Pre-cultured cells may be applied by pipetting or any other suitable method. Pre-cultured cells may be added at any suitable concentration. For example, cells may be added to each square centimetre of the construct at a concentration of at least 3500 cells/cm². Cells may be added to each square centimetre of the construct at a concentration from 3500 cells/cm²up to 100,00 cells/cm² or more. For example, at least 3500 cells/cm², 4000 cells/cm², 4500 cells/cm², 5000 cells/cm², 5500 cells/cm², 6000 cells/cm², 6500 cells/cm², 7000 cells/cm², 7500 cells/cm², 8000 cells/cm², 8500 cells/cm², 9000 cells/cm², 9500 cells/cm², 10000 cells/cm², 10500 cells/cm², 11000 cells/cm², 11500 cells/cm², 12000 cells/cm², 12500 cells/cm², 13000 cells/cm², 13500 cells/cm², 14000 cells/cm², 14500 cells/cm², 15000 cells/cm², 15500 cells/cm², 16000 cells/cm², 16500 cells/cm², 17000 cells/cm², 17500 cells/cm², 18000 cells/cm², 18500 cells/cm², 19000 cells/cm², 19500 cells/cm², 20000 cells/cm², 20500 cells/cm², 21000 cells/cm², 21500 cells/cm², 22000 cells/cm², 22500 cells/cm², 23000 cells/cm², 23500 cells/cm², 24000 cells/cm², 24500 cells/cm², 25000 cells/cm², 25500 cells/cm², 26000 cells/cm², 26500 cells/cm², 27000 cells/cm², 27500 cells/cm², 28000 cells/cm², 28500 cells/cm², 29000 cells/cm², 29500 cells/cm², 30000 cells/cm², 30500 cells/cm², 31000 cells/cm², 31500 cells/cm², 32000 cells/cm², 32500 cells/cm², 33000 cells/cm2, 33500 cells/cm², 34000 cells/cm², 34500 cells/cm², 35000 cells/cm², 35500 cells/cm², 36000 cells/cm², 36500 cells/cm², 37000 cells/cm², 37500 cells/cm², 38000 cells/cm², 38500 cells/cm², 39000 cells/cm², 39500 cells/cm², 40000 cells/cm², 40500 cells/cm², 41000 cells/cm², 41500 cells/cm², 42000 cells/cm², 42500 cells/cm², 43000 cells/cm², 43500 cells/cm², 44000 cells/cm², 44500 cells/cm², 45000 cells/cm², 45500 cells/cm², 46000 cells/cm², 46500 cells/cm², 47000 cells/cm², 47500 cells/cm², 48000 cells/cm², 48500 cells/cm², 49000 cells/cm², 49500 cells/cm², 50000 cells/cm², 50500 cells/cm², 51000 cells/cm², 51500 cells/cm², 52000 cells/cm², 52500 cells/cm², 53000 cells/cm², 53500 cells/cm², 54000 cells/cm², 54500 cells/cm², 55000 cells/cm², 55500 cells/cm², 56000 cells/cm², 56500 cells/cm², 57000 cells/cm², 57500 cells/cm², 58000 cells/cm², 58500 cells/cm², 59000 cells/cm², 59500 cells/cm², 60000 cells/cm², 70000 cells/cm², 80000 cells/cm², 90000 cells/cm², or 100000 cells/cm² or more.

After seeding cells onto the construct the construct may be subjected to conditions to lead to crosslinking and/or polymerisation of the cell support agent. For example, conditions that lead to the cell support agent forming a hydrogel who act to support the cells. For example, the seeded construct may be incubated for at least 1 hour at a temperature of around 37° C. Incubating the construct with the seeded cells may also allow for the attachment cells to the construct and/or cell support agent. In the case a construct with open channels, the cells may attach and therefore be hosted and maintained in the channels. In the case of a construct having a porous flexible polymer sheet as described herein the cells may infiltrate the pores of the flexible polymer sheet and attach therein. Therefore, the cells are hosted and maintained within the pores of the flexible polymer sheet.

In the examples, where the construct includes channel forming members within the channels, these are removed. For example, the channel forming members may be pulled from the construct to leave tubular or closed channels. The channel forming members may be removed by any suitable method such as by pulling using tweezers.

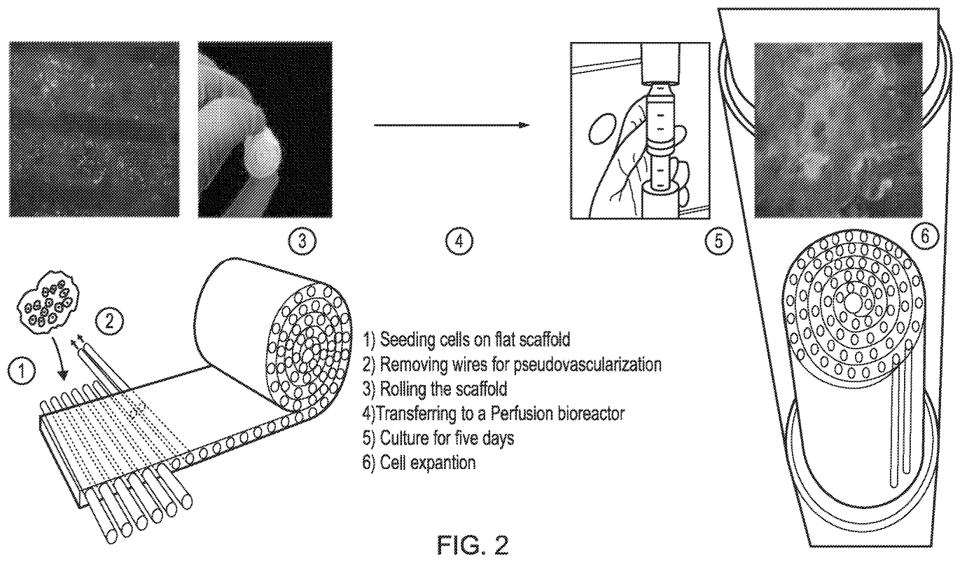

Once the seeded construct has been crosslinked, the construct is manipulated to form a 3D filled shape. For example, the construct may be rolled to form a filled cylinder as shown in FIG. 2. Manipulating the construct may be done by hand, for example using tweezers to hold and manipulate the construct. Other methods of rolling the construct will be known, such as using machines suitable for rolling textiles or paper. The shape of the manipulated construct may be selected based on the shape of the chamber of the bioreactor to be used. For example, for a cylindrical chamber the construct may be manipulated to be a filled cylinder. In the case of a cuboidal bioreactor chamber, the construct may be folded to be a solid cuboidal shape. That is to say the construct may be manipulated to be a shape that conforms with the shape of the internal volume of the chamber of the bioreactor to be used.

Once manipulated to the desired 3D structure the manipulated construct is disposed into the chamber of a bioreactor. For example, placed in the internal volume of the chamber. The construct may be sized so that the construct substantially fills the internal volume of the chamber.

Once the construct is disposed within the chamber the chamber may be sealed and cell culture media is perfused through the channels of the construct and through the perfusion bioreactor system (i.e. into and out of the chamber internal volume). The cell culture media may be perfused using any suitable system such as a pump system. For example, a peristaltic pump system.

In constructs having open channels and having a non-porous flexible polymer sheet the cell culture medium is perfused (i.e. flows) through the channels and is therefore in contact with the cells hosted within the channels. In the case of a construct having closed channels and a flexible porous polymer sheet, the cell culture medium perfuses (i.e. flows) through the channels and infiltrates into the pores of the porous flexible polymer sheet and thus into contact with the cells hosted in the pores of the porous flexible polymer sheet.

The perfusing media diffuses through the pores of channels to the body of the construct and provides cells constantly with oxygen and nutrients while simultaneously carrying away cellular metabolism waste. This may provide a replica of a capillary system in natural muscle which may enable cellular viability and proliferation at high densities.

The construct may be maintained in conditions suitable to allow cells to proliferate and/or differentiate. Such conditions may be selected based on the cell type being used. As an example, the construct may be maintained at a temperature of around 37° C.

Cell culture medium may be perfused at a rate of perfusion based on the number of channels in the construct. For example, at a flow rate of at least 0.1 μL/min per channel. For example as flow rate of at least 0.1 μL/min, 0.2 μL/min, 0.3 μL/min, 0.4 μL/min, 0.5 μL/min, 0.6 μL/min, 0.7 μL/min, 0.8 μL/min, 0.9 μL/min, 1 μL/min, 1.1 μL/min, 1.2 μL/min, 1.3 μL/min, 1.4 μL/min, 1.5 μL/min, 1.6 μL/min, 1.7 μL/min, 1.8 μL/min, 1.9 μL/min, 2 μL/min, 2.1 μL/min, 2.2 μL/min, 2.3 μL/min, 2.4 μL/min, 2.5 μL/min, 2.6 μL/min, 2.7 μL/min, 2.8 μL/min, 2.9 μL/min, 3 μL/min, 3.1 μL/min, 3.2 μL/min, 3.3 μL/min, 3.4 μL/min, 3.5 μL/min, 3.6 μL/min, 3.7 μL/min, 3.8 μL/min, 3.9 μL/min, 4 μL/min, 4.1 μL/min, 4.2 μL/min, 4.3 μL/min, 4.4 μL/min, 4.5 μL/min, 4.6 μL/min, 4.7 μL/min, 4.8 μL/min, 4.9 μL/min, or 5 μL/min.

The cells that may seeded onto the construct may be any cell type. The cells may be more than one cell type. The cells seeded onto the construct may be muscle cells or cells that can differentiate into muscles cells. Muscle cells refers to those cells making up contractile tissue of animals. Muscle cells are derived from the mesodermal layer of embryonic germ cells. Muscle cells contain contractile filaments that move past each other and change the size of the cell. They are classified as skeletal, cardiac, or smooth muscles. As used herein, the term “cells that can differentiate into muscle cells” refers to stem cells and muscle progenitor cells that can differentiate into muscle cells.

Muscle cells may include those cells normally found in muscle tissue, including smooth muscle cells, cardiac muscle cells, skeletal muscle cells (e.g., muscle fibres or myocytes, myoblasts, myotubes, etc.), and any combination thereof. Muscle cells may include myoblasts, myotubes, myofibrils, and/or satellite cells.

The cells may include adipose or fat cells. Adipose or fat cells include any cell or group of cells composed in a fat tissue, including for example, lipocytes, adipocytes, adipocyte precursors including pre-adipocytes and mesenchymal stem cells.

The cells may be derived from any source animal. As the constructs described herein may be for use in making comestible process the cells may not be derived from a human. In some examples, the cells may be derived from a bovine, ovine, equine, porcine, caprine, avian, fish, insect, crustaceans, cephalopod, mollusc and/or camelid animals. Preferably the cells may be derived from a bovine, porcine, avian and/or ovine animal. For example, the cells may be derived from a cow, pig, chicken, fish, squid, insect, oyster and/or sheep.

The cell culture medium that may be used in the methods described herein may be any suitable cell culture medium. The cell culture medium may be selected depending on the type of cell cells being cultured. Examples of culture medium that may be used include minimal essential medium (MEM, Sigma, St. Louis, Mo); Dulbecco's modified Eagle medium (DMEM, Sigma); Ham F10 medium (Sigma); Cell culture media (HyClone, Logan, Utah); RPMI-1640 culture media (Sigma); and chemical-defined (CD) culture media (which are formulated for individual cell types), such as CD-CHO culture media (Invitrogen, Carlsbad, Calif). The culture solution described above can be supplemented with auxiliary components or contents as needed. This includes any component of the appropriate concentration or amount required or desired.

The culture medium described above can be supplemented with auxiliary components or contents as needed. The culture medium may include one or more additives such as antibiotics, proteins, amino acids and/or sugars.

“Medium” and “cell culture medium” refer to a nutrient source used for growing or maintaining cells. As is understood by a person of skill in the art, the nutrient source may contain components required by the cell for growth and/or survival or may contain components that aid in cell growth and/or survival. Vitamins, essential or non-essential amino acids, trace elements, and surfactants (e.g., poloxamers) are examples of medium components. Any media provided herein may also be supplemented with any one or more of insulin, plant hydrolysates and animal hydrolysates.

“Culturing” a cell refers to contacting a cell with a cell culture medium under conditions suitable to the viability and/or growth and/or proliferation of the cell.

Perfusing the cell culture medium may include perfusing a first culture media and then subsequently perfusing on or more second cell culture medias. The first cell culture medium may be a cell culture medium that is for proliferating cells and may be referred to a proliferation medium.

Proliferation medium may be a medium comprising a source of nutrients, such as vitamins, minerals, carbon and energy sources, and other beneficial compounds that facilitate the biochemical and physiological processes occurring during expansion or proliferation of cells. The proliferation medium may comprise one or more carbon sources, vitamins, amino acids, and inorganic nutrients. Representative carbon sources include monosaccharides, disaccharides, and/or starches. For example, the proliferation medium may contain one or more carbohydrates such as sucrose, fructose, maltose, galactose, mannose, and lactose. The proliferation medium may also comprise amino acids. Suitable amino acids may include amino acids commonly found incorporated into proteins as well as amino acids not commonly found incorporated into proteins, such as argininosuccinate, citrulline, canavanine, ornithine, and D-stereoisomers. The proliferation medium may also comprise proteins such as foetal bovine serum albumin. The proliferation medium may also comprise antibiotics.

For example, the proliferation medium may be Dulbecco's Modified Eagle's Medium (DMEM) that may include 10% (V/V) filter sterilized foetal bovine serum and 1% (V/V) penicillin/streptomycin solution.

The cells may be maintained and cultured in proliferation medium for at least 10 hours. For example at least 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120 hours.

The cell culture media may then be changed to a second cell culture medium. The second cell culture medium may be a differentiation medium. Differentiation medium refers to a medium designed to support the differentiation of cells, that is, supporting the process of a cell changing from one cell type to another. The differentiation medium may include one or more amino acids, antibiotics, vitamins, salts, minerals, or lipids. The differentiation medium may include at least one carbon source such as a sugar. For example, glucose. The differentiation medium may include one or more proteins, amino acids or other additional acids. In some examples the differentiation medium may be high-glucose DMEM (97%) supplemented with 2% horse serum and 1% penicillin/streptomycin solution.

The cells may be maintained and cultured in differentiation medium for at least 10 hours. For example at least 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120 hours.

Cells cultured on the construct may be cultured to a concentration of at least 100,000 cells/cm² during the proliferation phase in the proliferation medium. For example, at least 100000 cells/cm², 150000 cells/cm², 200000 cells/cm², 250000 cells/cm², 300000 cells/cm², 350000 cells/cm², 400000 cells/cm², 450000 cells/cm², 500000 cells/cm², 550000 cells/cm², 600000 cells/cm², 650000 cells/cm², 700000 cells/cm², 750000 cells/cm², 800000 cells/cm², 850000 cells/cm².

After culturing in the differentiation medium cells may be striated along the channel. The cells may form myotubes. The cells may form straited myotubules along each channel. Without being bound by theory, mechanical force from sheer stresses caused by the fluid helps the striation of the cells.

The cultured construct removed from the chamber. If the construct is made using an edible polymer, the cells and construct may be formed into comestible product including the scaffold. For example, the cells and construct may be formed into a meat analogue.

A meat analogue (which may also be referred to an cultured, or in vitro meat) refers to a food product that is not produced by the slaughter of an animal, but has structure, texture, aesthetic qualities, and/or other properties comparable or similar to those of slaughtered animal meat, including livestock (e.g., beef, pork), game (e.g., venison), poultry (e.g., chicken, turkey, duck), and/or fish or seafood substitutes/analogues. The term refers to uncooked, cooking, and cooked meat-like food product.

The construct and cells thereon may be configured to mimic the taste, texture, size, shape, and/or topography of a traditional slaughtered meat. For example, multiple constructs including cells may be combined in order to form a structure similar to a cut of meat or a portion sized product. For example, bound together or compressed together. For example, constructs may be bound together by an edible adhesive such as transglutaminase. In some examples, a single construct, in a manipulated state, may be used to form a meat analogue.

The construct or multiple constructs and the cells cultured thereon may have further agents added in order to make the sensory properties, such as texture, taste, smell and visual properties, more similar to a meat. For example, one or more of fats, texturizers, bulking agents, thickeners, preservatives, flavour enhancers, antimicrobial agents, pH modulators, desiccants, vitamins, minerals, metals, slats, sweeteners, curing or pickling agents, colouring agents, or any combination thereof may be added to the constructs. Additional agents may be dispersed through a construct or multiple constructs via the channels formed therein.

As such, also provided herein is a meat analogue including an edible construct as described herein.

Any meat analogue produced may have the dimensions of a whole cut of meat. For example, a single construct may have at least one dimension (i.e. at least one of length, width or thickness) that is at least 10 cm. For example, a single construct in the shape of a cylinder may provide a meat analogue having a length of at least 10 cm. The thickness or diameter of such a construct may up to 50 cm. Such constructs would provide a meat analogue having dimensions similar to a loin cut of a cow.

When the construct is not edible, the cells may be removed from the construct. That is to say that the cells may be recovered from the construct. The cells may be recovered by applying a cell-support specific agent to the construct. A cell support specific support agent may be any agent that is capable of degrading the cell support agent. For example, the cell support specific agent is an enzyme. For example, trypsin or nattokinase. In specific examples the cell support specific agent is nattokinase. Nattokinase is a fibrin-specific enzyme derived from fermented soybeans. The nattokinase may be food grade nattokinase.

The cell support specific agent may be added to a construct that has been manipulated to be a flat sheet again, for example unrolled. The cell support specific agent may be applied at a concentration of at least 10 mg/ml. For example, the cell support specific agent may be applied at a concentration of at least 10, 20, 30, 40, 50, 60 or 70 mg/ml.

When the cell support specific agent has been applied, the cells are removed from the construct and suspended in a composition including the cell support specific agent. The recovered cells may then be separated from the composition for example by centrifugation or other known methods.

The recovered cells may then be used to produce a comestible product. The cells may be processed into a product such as a meat analogue. For example, the cells may be subjected to similar processes as used for producing products such as sausages or processed meats products, for example reconstituted meats such as baloney. For example, the cells may be emulsified, ground or minced and then formed into a product that resembles a cut of meat or a meat product. For example, processed cells may be moulded or shaped using any known methods. The cells may have agents added to help processing such as fats, binders or texturisers added. The cells may also have additional agents added in order to make the sensory properties, such as texture, taste, smell and visual properties, more similar to a meat. For example, one or more of fats, texturizers, bulking agents, thickeners, preservatives, flavour enhancers, antimicrobial agents, pH modulators, desiccants, vitamins, minerals, sweeteners, salts, metals, curing or pickling agents, colouring agents, or any combination thereof may be added to the cells.

### EXAMPLES

### Example 1

There is growing interest in cultured meat as a protein alternative [1], [2], [3]. Lab-scale production of cultured meat in its simplest form of muscle cells or co-cultures of muscle and fat cells has been achieved [1], however scaling-up the process to make a viable economic product is still challenging [2], [4]. Challenges to be overcome include ethical sourcing of raw materials, reducing cost of cell culture media, increasing protein yield for the muscle cell culture, and within the bioprocess itself improved energy efficiency and resource utilization and waste valorisation [5]. For increased process efficiencies and reduced environmental impact, bioreactors with higher cell densities allow a smaller culture volume [4] thus reducing space requirements, labour requirements to set up and harvest the cells, and the amount of raw materials to manufacture them. Operating costs will be also lower as smaller bioreactors requiring less power and utilities [6], [4]. Additionally, if such a bioreactor could be designed to produce tissue constructs that mimic muscle itself this would take the field a step closer to realizing full-cut cultured meat i.e. replicating the exact structure of skeletal muscle. Fortunately, mimicking the anatomy of skeletal muscle, in terms of blood supply and myofibre structure, in cultured meat production is inherently a way to address the challenge of having high cell densities in reactors; as skeletal muscles typically have cell densities of 10⁷ to 10⁹ cells/cm³ [7], [8], [9]; depending on a number of factors particularly age; then animal, and muscle type [9], [10]. This is considerably greater than the maximum cell confluency previously reported to be achieved in CSTR cultured meat reactors [11], [12].

Native skeletal muscle anatomy consists of several arrays of uniaxial, striated myofibres in conjunction with fat cells, fibroblasts, capillaries and veins [13]. The key point here is that a capillary is connected to most of the myofibres in a fascicle to provide blood perfusion [14], to provide the muscle cells with adequate oxygen and nutrients and also takes away cell metabolism waste [13], [15]. This structure is well-replicated in a channeled perfusion bioreactor (PFB) where the inlet media carries oxygen and nutrients, goes through the channels and nourishes the cells and the perfused flow carries out the wastes as in outlet. As a result, many researchers proposed this concept to mimic muscle tissue, and overcome the mass transfer barrier in any 3D tissue culture and achieve high cell densities [16], [17], [15]. Additionally, growing cells in 3D cultures, provides more in vivo-like environmental niches which may stimulate cells' proliferation and differentiation [18], [19]. Although the concept of a channeled PFB seems to satisfy the targeted milestones to have higher cell density and the structure of whole-cut meat, practical challenges are yet to be overcome; such as: making a large-scale homogenous porous scaffold [20]; seeding the cells uniformly on 3D scaffold [21]; and rapid and easy vascularization [22].

In this study, the aforementioned bottlenecks of the PFBs are addressed by introducing the CASP bioreactor. Moreover, this novel concept realizes the possibility of increasing the size of a 3D cultured tissue construct, thus taking a step towards producing a portioned-sized 3D piece of cultured meat. A fast, simple, controllable and scalable method to create uniform pseudo-vascularized spiral-wound scaffolds homogenously seeded with muscle cells (C2C12s) has been developed. Polycaprolactone (PCL) was used as a biocompatible and relatively cheap polymer for sheet casting [19, 23]. Although PCL is not edible and hence not the ultimate choice for a whole-cut cultured meat, its mechanical properties and ease of use makes it a practical choice. A uniaxial array of wires was used to channel a casted porous sheet of PCL and cells were encapsulated in fibrin inside the pores of this scaffold. Hence, by rolling the rectangular sheet into a full-bodied cylinder, a uniform scaffold with homogenous cell seeding and well-distributed channels is obtained. Increasing the length and the width of the PCL sheet leads to increase in diameter and height of the resulting cylinder respectively and is therefore highly scalable.

### 1. Material and Methods

All materials were purchased from Sigma Aldrich and used as supplied, unless otherwise stated.

### 1.1. Scaffold Fabrication

Polycaprolactone (PCL, Sigma-Aldrich 440744, UK, Mₙ 80,000, pellet size ˜3 mm) was dissolved in acetone (Sigma-Aldrich 179124, Germany, ACS reagent≥99.5%) by 8.5% W/V. NaCl (Sigma-Aldrich S7653, USA) particles were grounded by a pestle and mortar, sieved to less than 125 μm and added (as 500% w/w of solid PCL) to the PCL-acetone solution. This solution was then heated to 45° C. and agitated properly until a uniform solution was obtained.

A long thin wire (tinned copper wire, SWG¹ 30, 0.315 mm diameter, Scientific Wire®, TC0315-500, UK) was wrapped around a flat glass sheet (to make a flat parallel array). Two laser etched PTFE supports glued to the edges of the glass sheet helped to keep a constant 1 mm interval between the wires; and also 0.5 mm space between the top of the wires and the surface of the glass sheet. ¹Standard Wire gauge

The polymeric solution was poured on the glass sheet. A glass rod wrapped by 1 mm thick wires (on each end) was used to cast a uniform 1 mm thick polymeric sheet on the glass surface. The polymeric solution completely enveloped the wires. After making the flat sheet using the wire rod, 10 see was given to the film to stiffen a little bit and then it was put inside a water bath (room temperature, 20° C.). The water in the water bath was changed every 12 hours (h) for one day and then the solid scaffold was left dry at room temperature. The wires were not removed.

### 1.2. C2C12 Maintenance Culture

The immortalized mouse myoblast cell line C2C12 (ECACC 91031101) were cultured in 75 mL t-flasks. The initial seeding density was 5,000 cells/cm² and the media was high glucose Dulbecco's Modified Eagle's Medium (DMEM; Sigma-Aldrich D5796) plus 10% (V/V) filter sterilized fetal bovine serum (FBS; Gibco™, Thermo Fisher Scientific 10270106) and 1% (V/V) penicillin/streptomycin solution (P/S; Sigma-Aldrich P4333). This media is referred to as “proliferation media” through the rest of these examples. Cells were incubated at 37° C. and in 5% CO₂ for 2-3 days until they reached 80% confluency [19].

### 1.3. Cell Seeding on PCL Scaffolds

PCL scaffolds (with the wires in situ) were cut into desirable dimensions. Prior to cell seeding, scaffolds were sterilized with 70% EtOH for 1 h, washed twice with PBS and DMEM, and kept in DMEM until use.

A 20 mg/mL solution of fibrinogen (Fibrinogen from bovine plasma, F8630, Sigma-Aldrich) was prepared in high glucose DMEM and filter sterilized (0.2 μm). This solution was pipetted on the scaffolds (half of the volume of the scaffold with any desirable size). Required amounts (based on the desirable seeding density) of pre-cultured cells (refer to section 2.2) were suspended in a specified volume of proliferation media (half of the scaffold's volume), and thrombin (Thrombin from bovine plasma, T7326, Sigma-Aldrich) was added to it in a manner that the solution had 4 U/mL of thrombin. This solution was pipetted on the scaffolds immediately after fibrinogen. The scaffolds were then incubated for 3 h to let fibrinogen clot. The wires were then removed from the scaffolds leaving hollow channels inside them. This seeding protocol was used preceding all cultures on scaffolds. It is notable that by using the protocol above, the fibrinogen and thrombin concentrations in the final fibrin hydrogel are 10 mg/mL and 2 U/mL respectively.

ChemoMetec NC-200 cell counter was used for counting the number of the cells before seeding scaffolds.

### 1.4. Investigating Seeding Efficiency on Unrolled Scaffolds and Spiral-Wound Scaffolds

To investigate any possible adverse effect of mechanical rolling of scaffolds on the seeded cells, two groups of samples were chosen to compare the seeding efficiency between flat (unrolled) scaffolds and scaffolds after being rolled. As the first group, square sheets of PCL (1 cm×1 cm) were cut and seeded with 50,000 cells encapsulated in fibrin (n=3, N=3). They were stained with fluorescein diacetate/propidium iodide and imaged using an Evos M5000 microscope. As the second group, rectangular straps of PCL scaffolds were cut (1 cm×10 cm), sterilized and seeded with cells encapsulated in fibrin similar to group one, with initial cell density of 50,000 cells/cm² of the scaffold. These scaffolds were then rolled and unrolled once (using hands and tweezers), cut to flat 1 cm×1 cm samples by a sharp blade and gone through staining and image processing similar to the first group.

### 1.5. Static Cell Culture

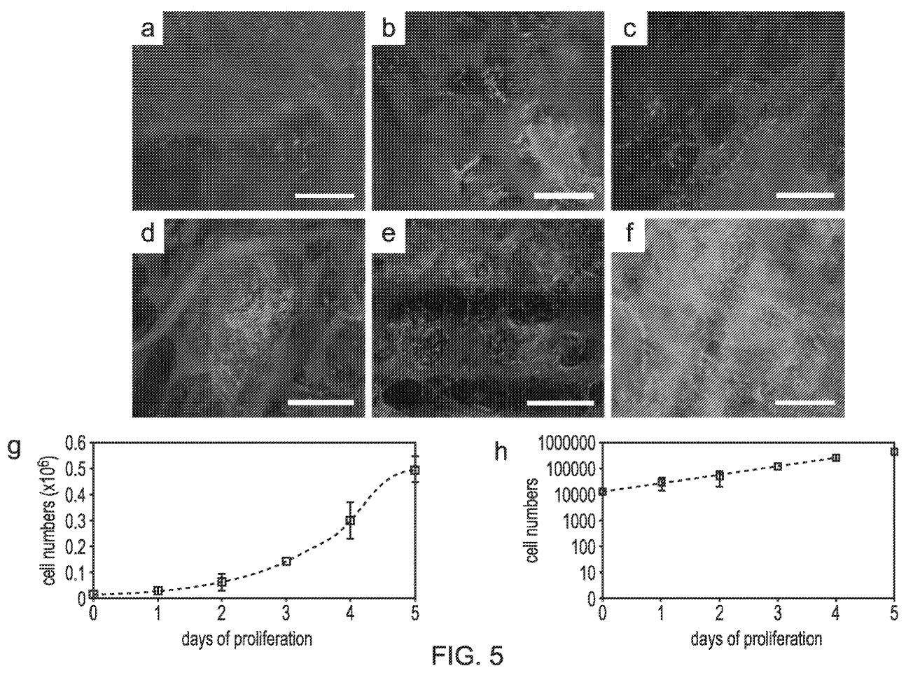

Rectangular straps of PCL scaffold were cut (4 cm×10 cm), sterilized and seeded with cells encapsulated in fibrin as described in section 2.3, with initial cell density of 50,000 cells/cm² of the scaffold. The scaffolds were then rolled to full-bodied cylinders with 8 mm diameter and 4 cm height. They were put inside a 2 ml sterile syringe cylinder (spec) with inner diameter of ˜8 mm (see FIG. 5) and cultured inside a Petri dish containing 30 mL proliferation media (3 days). After 72 h in culture, the scaffold was subjected to sampling, staining and imaging protocols. (n=3, N=2)

### 1.6. Dynamic Cell Culture

Seeded and rolled scaffolds were prepared as described in section 2.5. A 2 mL syringe cylinder (BD Plastipak) connected with a P3D-6 inlet part was used as a perfusion chamber for the scaffold (image in supplementary data). Complete media (30 mL) was perfused through channels of the scaffold with 1 μL/min per channel as the volumetric flowrate. Scaffolds were taken out of the reactor on day 3 and were subjected to sampling, staining and imaging protocols. (n=2, N=2)

### 1.7. Scale-Up of the CASP Construct and Bioreactor

Rectangular straps of PLC scaffold with different sizes i.e. 0.5 cm×10 cm, 1 cm×10 cm, 1 cm×30 cm, and 2 cm×90 cm (the latter being made from 7 straps of 2 cm×13 cm dimension) were prepared as described in section 2.3. They were seeded with 50,000 cell/cm² and rolled to the spiral-wound structure with 0.5 cm height and 8 mm diameter, 1 cm height and 8 mm diameter, 1 cm height and 13 mm diameter, and 2 cm height and 24 mm diameter respectively. The first two cylindrical scaffolds were put inside EBERs P3D-6 perfusion chamber while the medium-size scaffold was mounted in EBERs P3D-10 perfusion chamber. Omnifit EZ chromatography column (006EZ-25-15-AA) was used as the perfusion chamber for the biggest scaffold. The fluid flow rate was 0.05 cm³min⁻¹ in two first reactors (100 channels), 0.1641 cm³min⁻¹ in the medium-sized reactor (300 channels) and 0.45 cm³min⁻¹ in the biggest reactor (900 channels) leading to equivalent flowrate per channel (0.55±0.05 μL/min) in all scaffolds.

Complete media (30 mL) was perfused throw channels of the scaffolds with different volumetric rates as described (90 mL of media for the biggest scaffold). Flow-through oxygen sensors (PreSens, FTC-SU-PSt3-S) were used to measure oxygen concentration in inlet and outlet. Scaffolds were taken out of the reactor on day 3 and were subjected to sampling, staining and imaging protocols (section 2.13).

### 2.8. Analyzing Cell Viability

### 2.8.1. Simultaneous Cell Counting and Viability Assessment of Re-Suspended Cells

A 50 mg/mL enzymatic solution of nattokinase was made by mixing the powder content of Nattokinase capsules (Best Naturals, 2000FU, 100 mg active ingredient per capsule) in PBS and then filter sterilising it through a 0.2 μm syringe filter (the solution was used within 3 hrs). For cell counting, scaffolds were cut to 1 cmx 1 cm squares and put inside a well-plate. 1 mL of the enzymatic solution (at 37° C.) was added to each sample. Samples were incubated at 37° C. for 15 min and mixed gently by pipetting every 5 min. Nattokinase lyses fibrin and resuspends the cells in a solution. A portion of this cell solution was examined by ChemoMetec NC-200 cell counter to assess cell numbers and viability. ChemoMetec cassettes use acridine orange and DAPI to stain total/dead cells respectively, the cells are then automatically imaged and the image is processed to provide the cell counts. As the last step, after treatment, scaffolds were stained and imaged to investigate the efficiency of the enzyme treatment (by assessing the percentage of un-detached cells).

### 2.8.2. Simultaneous Cell Counting and Viability Assessment by Live/Dead Staining the Scaffolds

After sampling (or after culture for readily cut to 1 cmx 1 cm scaffolds), Hoechst 33342 (ThermoFisher Scientific, H21492) [1:2000 dilution of Hoechst stock solution (10 mg/mL in deionized water) in PBS] was used for nuclei staining proceeding by fluorescein diacetate (FDA; Acros Organics 191660050) and propidium iodide (PI; Fisher, 11425392) protocols for live/dead staining.

Samples were washed with PBS and incubated in Hoechst for 10 minutes at room temperature. They were washed with DMEM afterwards and went through live/dead staining.

An aliquot of 8 μL of FDA stock solution (5 mg/mL FDA in acetone) was added to each 5 mL of DMEM (without FBS) and PI (2 mg/mL PI stock solution in PBS) was added at 1:100 dilution ratio. Cells were incubated in the formerly described FDA/PI staining solution for 5 minutes at room temperature, washed twice with PBS and imaged inside a 24-well plate while all liquid media was aspirated.

Fluorescent microscopy was performed by an Evos M5000 microscope. Cell counting and image processing was performed using ImageJ software. These results were cross-checked with the results of the previous method, for more certainty. Doubling time calculations were applied as explained by Hanga [11].

### 2.9. Proliferation and Viability on Flat Sheets

Square sheets of PCL (1 cm×1 cm) were cut and seeded with 50,000 cells. They were put inside low-attachment 24-well plates, supplemented with 0.5 mL of proliferation media and incubated (37° C., 5% CO2) for 5 days. Three samples were stained and imaged immediately after seeding (day 0 of the results). Simultaneous cell counting and viability assessment was done using two different methods explained in previous section. Samples were in triplicate for each assessment and tests were repeated 3 times.

### 2.10 Proliferation and Viability in Spiral-Wound Constructs (Dynamic Culture)

Rectangular straps of PCL scaffolds were cut (1 cm×10 cm), sterilized and seeded with cells encapsulated in fibrin as described in section 2.3, with initial cell density of 50,000 cells/cm² of the scaffold. The scaffolds were then rolled to full-bodied cylinders with 8 mm diameter and 1 cm height. Ebers-P3D6 chambers were used as PBRs. Two reactors were set-up for each day of proliferation (n=1, N=2). They were opened on each day of proliferation, sampled, stained and imaged for cell counting (and viability).

### 2.11. Differentiation on Unrolled Scaffolds

Cells were seeded on scaffolds exactly as described before and proliferated for 3 days (as at this time point confluency have reached a point when signs of myoblast fusion were detected). After 72 h of proliferation, the proliferation media was changed to differentiation media; i.e. high-glucose DMEM (97%) supplemented with 2% horse serum (HS; Sigma-Aldrich, H1270) and 1% P/S. Cells were incubated under the same condition for 7 days and 14 days, with differentiation media being renewed every 72 h. Scaffolds were subjected to staining and imaging afterwards as previously described.

### 2.12. Differentiation in Spiral-Wound Constructs Inside the CASP Bioreactor

An 8-mm-diameter and 5-mm-high cylindrical scaffold was prepared and cultured in proliferation media as described previously. After 120 h of proliferation the media bottle was changed into differentiation media (30 mL). PBR was run by differentiation media for 7 days (12 days in total considering the proliferation time) with media changing every 72 h. Per-channel volumetric flowrate was 0.5 μL/min on proliferation phase and 1 μL/min on differentiation phase. The scaffold was taken out of the reactor on day 7 of differentiation and were subjected to sampling, staining and imaging protocols as described previously.

### 2.13. Analytical Methods

### 2.13.1 SEM

Scanning Electron Microscopy (SEM) was used to investigate general morphology and characteristics of the porous PCL sheet. After complete drying by air at room temperature, samples were cut by scissors and wires were removed using a pair of tweezers. Some samples were snap-frozen in liquid nitrogen and cut by a sharp blade to provide a cross-section of channels' walls. Samples were coated with 50 nm of gold in a Sputter Coater (Edwards, S150B) for 2-3 min, and imaged by the SEM (JEOL, JSM-6480LV).

### 2.1.3.2 Porosity

Porosity of the scaffold was assessed by the equation 1 [24]. Briefly, 1 cm×1 cm squares of scaffold (with wires removed) were weighed and their volume was calculated (the empty volume of the channel spaces was subtracted). Multiplying the calculated volume to the density of PCL, gives the mass of a non-porous scaffold, while the experimental mass (W) has considered the empty spaces of the pores. Proportion of these two masses helps to find the porosity as in equation 1.

$$ε = 1 - \frac{W}{ρ \times V} \qquad ( Eq . l )$$

where ε (%) is porosity, W (gr) is the weight of the scaffold, V is the total volume of the rectangular scaffold (cm³) and ρ (gr·cm⁻³) is the density of PCL.

2.13.3 Sampling from Reactors

Reactors were opened for sampling, the scaffold was unrolled and it was cut in a way to acquire samples from different layers of the roll (inner parts, middle and outer layers). Also, if the scaffold was longer than 1 cm in perfusion length, different samples from inlet, outlet and middle parts were taken. Details of this are explained in supplementary data.

### 2.13.4 Detecting Dissolved Oxygen Levels

Dissolved oxygen levels from inlet and outlet of the reactor was read using flow-through sensors (PreSens, FTC-SU-PSt3-S) connected to an optic oxygen meter (Fibox4, PreSens) roughly every 12 h.

### 2.13.5. Data Analysis and Statistics

Number of the experimental repeats is stated by (n) while (N) represents number of the replicates. Error calculations are based on mean standard deviation (SD) unless otherwise stated. Statistical method for significances were assessed by one way-ANOVA. Differences were considered to be significant when p<0.05.

### 2. Results and Discussion

### 2.1. CASP Scaffold Fabrication

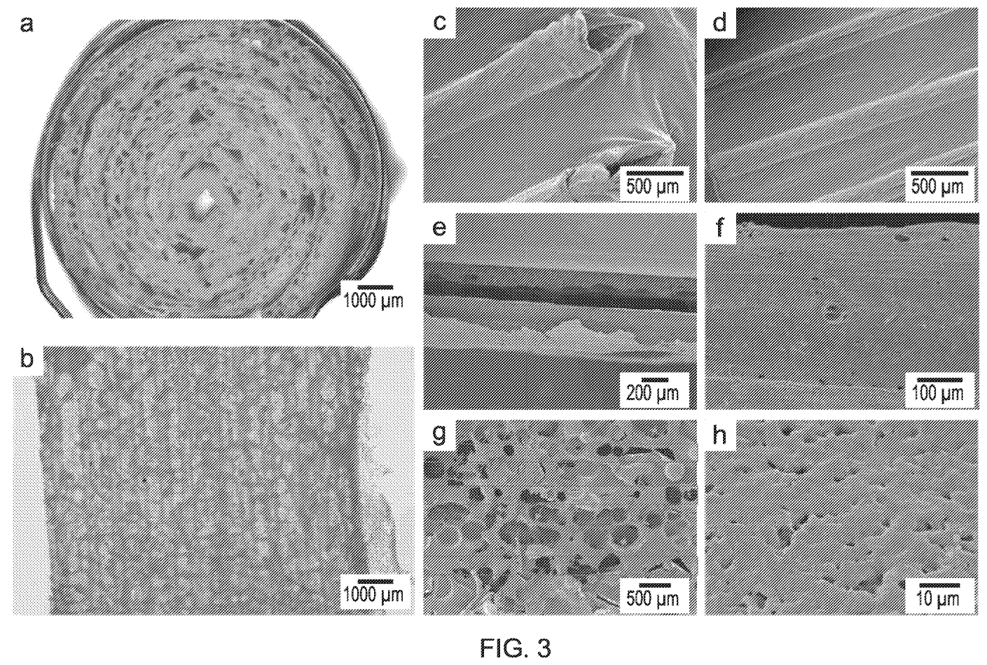

A white (semi-transparent) porous sheet of polycaprolactone was formed after solvent leaching from polymeric solution (FIG. 3a). The channels remained intact after removing the wires (FIGS. 3a and 3c), with an average channel interval of 956±190 μm and an average channel diameter of 306±33 μm. The sheets had an overall porosity of 85% with an average pore size of 375±150 μm. The average thickness of the sheet was 504±73 μm. Multiple macrovoids were found on channel walls as shown in FIG. 3 e, f and g. The diameter of these holes were different form tens of micrometers to fractions of micrometer. Interconnection between pores in the bulk of the scaffold, and also pores and channels were observed. Lastly, two different surface structures were observed in the scaffold. As shown in FIG. 3 g and h, the PCL film had two different surface structured related to the fabrication method; one was exposed to air and less porous (FIG. 3h) and the other one was in touch with glass and more porous (FIGS. 3a and 3g). The sheet had mechanical integrity such that it could be rolled and unrolled without cracking or loosing physical integrity (FIG. 3b).

PCL was chosen as the polymeric supporting scaffold in this study because of its several desirable characteristics. First and foremost because it is very flexible [25] and hence suited for rolling into the desired spiral-wound structure [26], [27]. It is also cheap and FDA approved [25], meaning that while it is used here as model for edible construct, the PCL construct could be used in regenerative medicine applications as well. Finally, PCL is recommended by other researchers for musculoskeletal tissue engineering [6], [28]. Although there are several methods for casting porous PCL sheets, solvent casting accompanied by particle leaching was chosen over the others because of its ease of use, rapidness and also ability to control pore structures [28], [20]. Guarino 2007 [20] states there is a barrier of 3 mm as thickness of the phase-inversion-made scaffolds. Acetone and water were used as benign and comparatively cheap solvent and anti-solvent respectively. A shrinkage in casted sheet thickness was observed (from 1 mm to ˜0.5 mm) which is a common phenomenon in solvent extraction process [25]. This shrinkage had no adverse effects on applicability of the scaffold as can be seen in the following sections, and moreover a thinner sheet is more favorable for mass transfer phenomena. The resulting diameter of the channels (306±33 μm) and also the space between them (956±190 μm) followed the design as desired (315 μm and 1000 μm, respectively). Accordingly, the sheet casting process was easy, rapid, highly controllable and highly scalable in 2D which means high control and scalability in the resulting rolled 3D CASP scaffolds.

The sheet showed favorable elasticity and could be rolled to a full cylindrical spiral-wound scaffold as desired. Iwasaki (2008) previously applied rolling a porous sheet of PCL [23], and Frost et al. (2018) [26] have used a rolled, channeled PCL sheet. Frost et al. used a layer-by-layer electrospinning approach to fabricate the scaffold. Electrospinning gives very small pores which are smaller than the actual size of the cells and as a result the bulk of the scaffold cannot be used for culture [26], [27]. Accordingly, in the study by Frost et al [26], the channels were used to grow the cells, and the porous electrospun bulk of the scaffold was used for media perfusion [26]. As a result, a very small fraction of the scaffold volume (just-7%) could be used for cell culture. In this study the scaffold design allows culture throughout the scaffold volume. Jeffries and Wang (2013) [27] by employing a method similar to Frost made a channeled scaffold with 50% culturable volumes, while in this study 75% of the volume is available for cell culture. The current study is the first to develop such a scalable vascularisation method to be used in a PFB.

The pores present in the channel walls (shown in FIG. 3 c and d) guarantee the media transfer from the channels to the porous scaffold. The wall also protects the cells from high sheer stresses of the fluid flow [29].

A previous study by (Tang 2004) [30] demonstrated the presence of two different surfaces, one with coarse and another with fine pores, in a cast sheet. In this study, although the pores in the less porous surface are considerably smaller than the pores in the bulk of the scaffold (1-10 μm compared to ˜500 μm pores), they are still bigger than 0.2 μm which is the pore size of sterilization filters used for the culture media and ensure passing of the larger components of media, i.e. the proteins. It is thought that the less porous surface increased the mechanical resistance, contributing to the high flexibility and stability of the cast sheet.

A number of researchers have carried out tissue culture in PBFs utilizing a channeled hydrogel scaffold [31], [32], [33], [34]. While these examples demonstrate feasibility, there is a lack of scalability (as noted by the authors themselves [33]) as hydrogels are not rigid enough to maintain form at larger scales of more than several mm, [35], [22]. Furthermore, hydrogels usually shrink and change form [32], [36]. This leads to deformed channels and scaffold geometry in the mentioned studies, while in this work the solid PCL scaffold (similar to a backbone) reinforces and supports the hydrogel.

As another advantage, while in most bioprinted scaffolds vasculature lumen occupies a big percentage of the scaffold volume e.g. 80% of the scaffold (PBF chamber) in Pourchet et al 2019 [32]; in this current study, channels occupied only 14% of the reactor chamber resulting in a more available space for cells to expand.

### 2.2. Cell Seeding

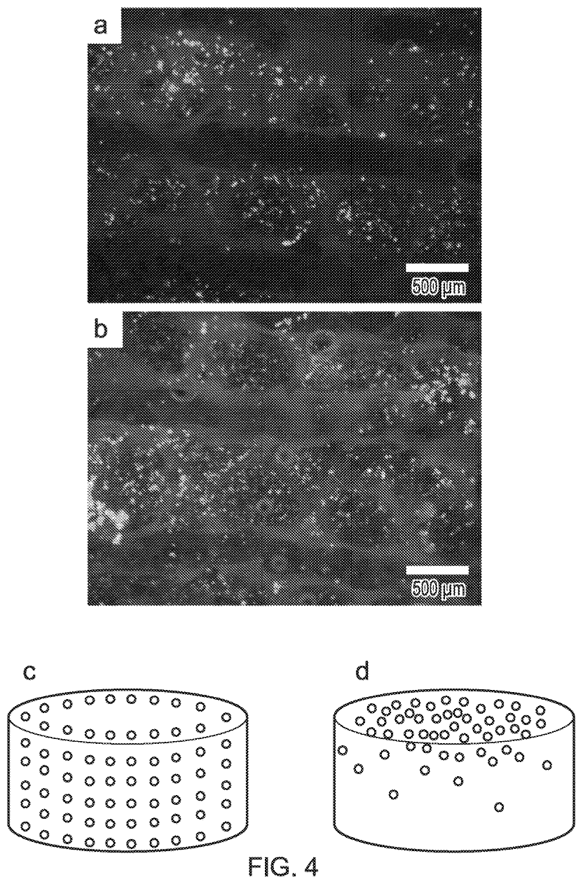

A comparison between cell seeding on rolled spiral-wound scaffolds and the unrolled sheets was performed. Accordingly, 16,355±3,225 cells of the initial 50,000 cells were attached on each cm² of unrolled 1 cm×1 cm sheets with viability of 88.35±5.1% leading to an average of 14,447 viable cells/cm² (equivalent to 28.89% seeding efficiency). On the other hand, 31,833±10,351 cells of the initial 50,000 were successfully seeded on the long strip scaffold, counted after being rolled and un-rolled, meaning that the chance of cells leaking out of scaffold is noticeably less when the ratio of surface to periphery is bigger in a scaffold. Viability of the cells was assessed to be 75.53±17.63% after rolling and unrolling which is a little less than the viability of unrolled scaffolds. However, in total, a higher number of viable cells are seeded successfully on the rolled scaffold i.e. 24,043 viable cells/cm² (equivalent to 48.09% seeding efficiency). It was seen that the seeding efficiency was improved at the larger scale. This is because cells are added to scaffolds in liquid format and it takes time for fibrin to cross-link. In the meanwhile, the liquid can escape (leak) from scaffolds periphery. This chance is less in scaled scaffolds as the ratio of periphery to surface is decreased. FIG. 4 compares these seedings on 1 cm×1 cm unrolled scaffolds with seeding on 1 cm×10 cm rolled scaffolds.

Although previous studies[37], show a good efficiency in cell seeding in PFBs (non-channelled, 75 to 94%), Meidhof (2010) [38] and Marin (2017) [21] proved that the aforementioned efficiency is not valid in perfusion cell seeding of channelled scaffolds; Marin (2017) states that perfusion cell seeding in channelled scaffolds lead to an efficiency next to zero (because the cells tend to just pass through the channels and not diffuse in the bulk of the scaffold) and recommends static cell seeding or rotational seeding for these scaffolds. While only 10-25% of efficiency in static cell seeding is reported [37], reaching a uniform distribution of cells is also a challenge in this method. Rotational seeding, as another mentioned method, can be used to seed channelled scaffolds for better efficiencies and distributions; however, the seeding efficiency is often not very higher than static seeding and the duration may be up to 24 hrs [37]. Furthermore, the process and the required equipment would be much more complex and energy-consumptive than the method in the present study. Our novel seeding method has doubled efficiency compared to static cell seeding with the additional advantage of uniform cell distribution (FIG. 4 c). As another advantage, seeding in the current study is a 3 hr process, while static or perfusion cell seeding is reported to last up to two days to reach the optimum capacity [38].

### 2.3. Cell Viability and Doubling Time on Flat Sheet Scaffolds.

Briefly, small squares of flat scaffolds were grown in proliferation media for 5 days. Each day, 3 samples were taken and gone through staining and imaging processes. To cross-check the validity of cell counting, in parallel, a set of scaffolds underwent the cell suspension process using nattokinase enzyme and counted by a cell counter. As a result, both assays showed the same trend, and the cell numbers counted by both methods were similar. Cytocompatibility of the scaffold is also proved this way. Using the combination of fibrin and PCL has been reported cytocompatible by Pankajakshan (2008) [39] and Kang (2011) [40] previously. FIG. 5 shows qualitatively how cells grow through a 5 day proliferation period and the graph quantifies it. As can be seen, the cells enter the exponential growth phase from day 1 onwards. Furthermore, the majority of the cells are live cells (green) from day 3 onwards (97% viability). Doubling time was calculated for proliferation on the flat sheets and the resulting doubling time (21.88 h) falls between the range reported by other publications. According to many reports the doubling time for C2C12s is around 20 h in well plates [41], [42].

### 2.4. Cell Viability and Doubling Time in the CASP Bioreactor

Two reactors (N=2) were used in parallel for this experiment. The dimensions of the cylindrical spiral-wound scaffolds were 10 mm in height and 8 mm in diameter. The seeding density was the same as the experiment for unrolled sheets (see section 3.3) to provide the possibility of comparison. On each day, the reactors were opened and sampled from different layers (refer to the supplementary data). Since the scaffolds couldn't be reused after sampling, 10 reactors were used in total to open a pair on each day. For each time point, one of the opened reactors was analyzed with the cell in situ by staining and image processing and the other analysed by removing the cells using nattokinase enzyme followed by cell counting, and the data were then compared.

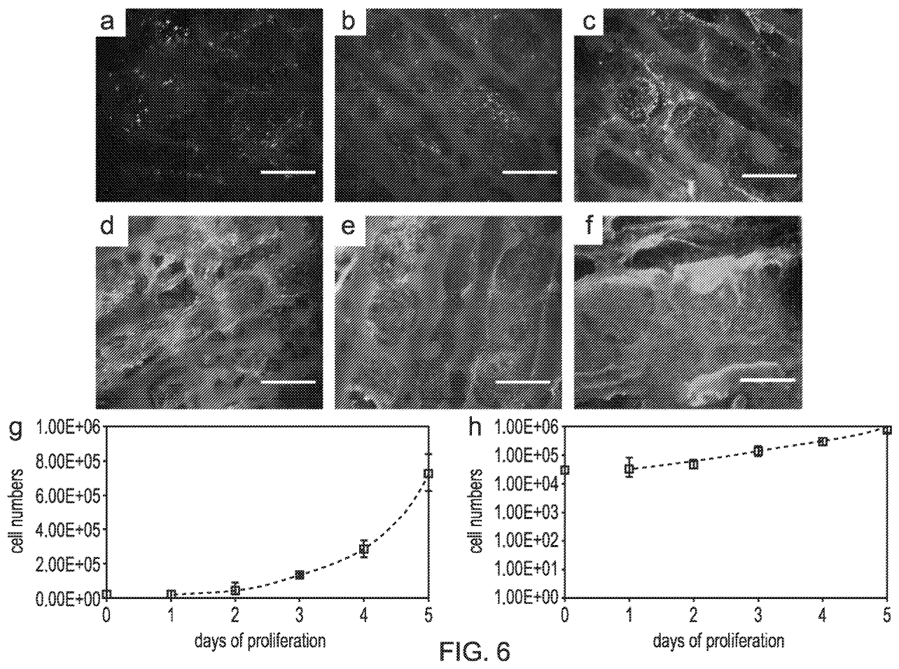

FIG. 6 shows qualitatively and quantitatively how cells grew over the 5-day proliferation period. Similar to the flat sheets, the majority of the cells were live cells (green) from day 3 on (98% viability). Doubling time was calculated and found to be 20.4±0.63 hours. Viability of the cells and the growth rate were comparable to unrolled scaffolds. The growth rate was 0.0023 hr⁻¹ higher for the CASP scaffold (equal to 1.46 hr less doubling time), supporting the idea that the structure here more closely replicated the in-vivo niche compared to the unrolled sheet. The porous PCL channel walls and the hydrogel (fibrin) in this design acted as protective barrier to direct shear while allowing adequate media supply, similar to the concept of a hollow fiber bioreactor [29]. There was no meaningful difference in cell numbers at the inlet and outlet of these reactors. The number of cells in some samples were up to 10% higher in inlet while in the other samples it was vice-versa (up to 10% higher in outlet). These differences were not out of the variance range of the samples within one reactor. However, there was a higher cell growth on the inner layers of the scaffold witnessed in some tests. In one of the reactors, after five days of proliferation, cell numbers were 940,000±85,000 cells/cm² in inner layers of scaffold; while this was 719,000±50,000 in middle and outer layers. These irregularities were not often observed and could be related to errors of manual cell seeding. The small error in number of seeded cells can become a big figure after several days of exponential cell expansion.

The main benefit of culture in CASP bioreactors compared to unrolled scaffolds, 850,000±150,000 cells per square cm of scaffold could be seen on day 5 of proliferation; while on flat sheets (unrolled scaffolds) under static conditions a maximum cell numbers of 400,000±100,000 was observed. This was likely due to the diffusion-only transport of oxygen and nutrients under stationary conditions that limited the growth of cells. On the contrary, in the CASP bioreactor, there is both axial convection of the media through the channels and the radial diffusion through the growing layers of cells.

In comparison to previous studies, with similar seeding densities of 10⁶ cell/cm³, less than 2.0×10⁶ myoblasts per mL were reported by Verbruggen et al (2018) [12] after 5 days of culture in CSTR; while 1.4 to 2×10⁷ were obtained in our study, i.e. an order of magnitude higher. As another comparison, after 5 days of culture, a 33-fold expansion was obtained in current study while Hanga et al [11] report a 28-fold increase after 9 days of culture in a CSTR for bovine myoblasts and not C2C12s.

### 2.5. Comparison Between Static and Dynamic Cell Culture in Spiral-Wound Constructs

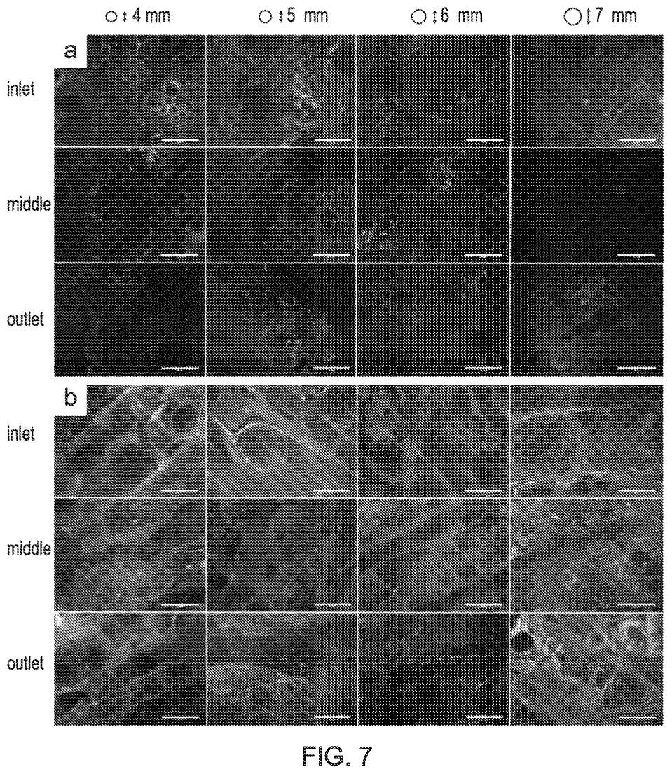

A comparison between static and dynamic culture of a scaffold with 40 mm perfusion length and diameter of 8 mm was carried out. A necrotic core in the static culture was observed (FIG. 7. a), while the dynamic culture (FIG. 7. b) showed high viability throughout the construct. This shows the necessity of perfusion flow in long scaffolds. Pseudovascularisation made it possible to have perfusion lengths of cm size which was hard to achieve in similar studies [43], [16], [31], [17]. Among these similar studies, those who didn't applied channeling in PFBs could not go beyond perfusion lengths of several millimeters [43], [38]. Among those who used channeled scaffolds, Radisic et al [16] reported a channeled scaffold with perfusion lengths of 2 mm at maximum. Aspect ratio of laser channeling, limitations of static cell seeding and quick oxygen drops caused by high (10⁸ cells/mL) cell densities were within the factors of limitation for the perfusion length in the mentioned study. Rnjak-Kovacina et al [31] report a 15 mm perfusion length in their study. We hypothesize that inadequate mechanical properties of the hydrogel scaffold would be a barrier to increase its height and perfusion length. Pourchet [32] et al by using the same hydrogel (fibrin) report a perfusion length of 20 mm. They also validate our aforementioned hypothesis that cell-enveloping hydrogels are not rigid enough to maintain high perfusion lengths. Hence, to the best of our knowledge, our study has improved the possible reported perfusion lengths in a tissue engineering PFB by 100%.

Rnjak-Kovacina et al [31] also compare cell growth in channeled and unchanneled scaffolds under static culture and observe no difference between these two under static culture (necrosis could be seen equally in both). This backs our observation of necrotic core in channeled scaffolds under static culture, meaning that perfusion of the media is also a key factor for cells to grow equally in all parts of the scaffold and stop necrosis.

Although the perfusing fluid is exerting sheer stresses on cells adjacent to the channel walls, no masses of dead cells were observed along these channels (unless in some defected areas, FIG. 7. b). We hypothesize that the porous polymeric channel walls and the hydrogel are acting as a shield between the cells and direct sheer stresses from the perfusion flow. Although in some areas the channel walls are not present due to channels defects and fabrication imperfection. In these areas masses of dead (red) cells could be noticed (FIG. 7. b).

### 2.6. Scale-Up of the CASP Bioreactor

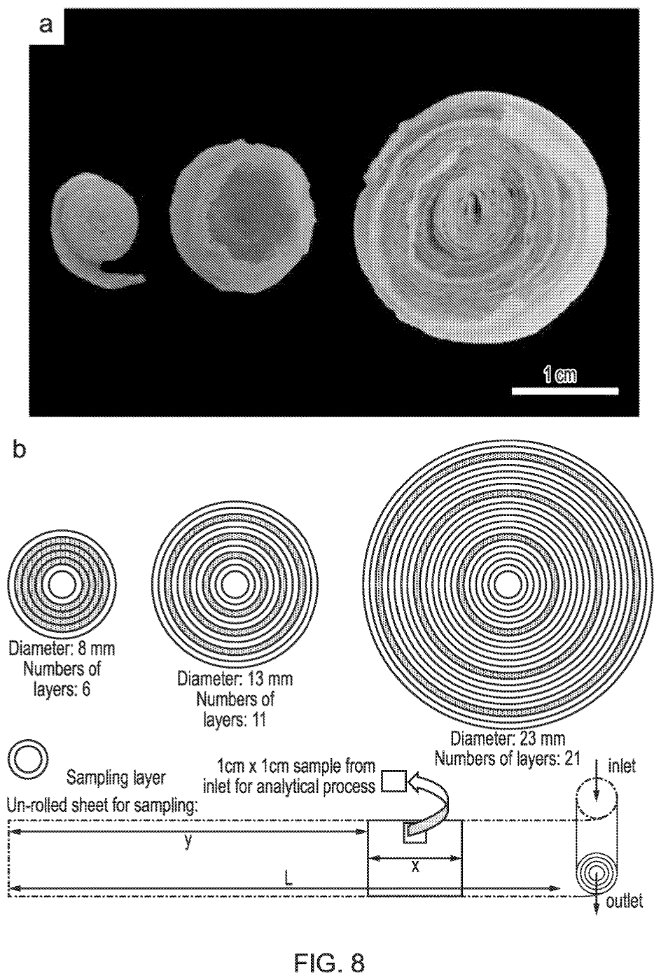

Scaling-up was performed stepwise, twice by increasing the diameter from 8 mm to 13 mm and then 23 mm while maintaining increasing the length from 5 mm to 10 and then 20 mm respectively. Accordingly, the volume of the scaffold was roughly 6-fold in each scale-up, leading to a 36-fold increase in size in total (FIG. 8 a, l, m and n). Additionally, scale up was performed with an 8 mm diameter and 40 mm long scaffold as a proof of further scalability in the perfusion length. At all stages of scale up, high viability was observed in all constructs (more than 97% viability in all samples FIG. 8. c to k), qualitatively demonstrating the proposed method was scalable without detriments to the cell viability. Similarly, increasing the perfusion length from 1 cm to 4 cm had no adverse effect on cell growth; 158,000±61,000 cells per cm² in 4-cm-long scaffolds compared to 143,000±47,000 cells per cm² in 1-cm-long scaffold on day 3 of the proliferation (p<0.05, one way ANOVA)—the slight difference could be the result of slightly different seeding efficiencies, sampling errors, cell counting errors, etc. which is comparable to the statistical error of the methods. Furthermore, no meaningful difference between the viability of cells or total cell numbers was found at the inlet and outlet of this system (p<0.05, one way ANOVA).

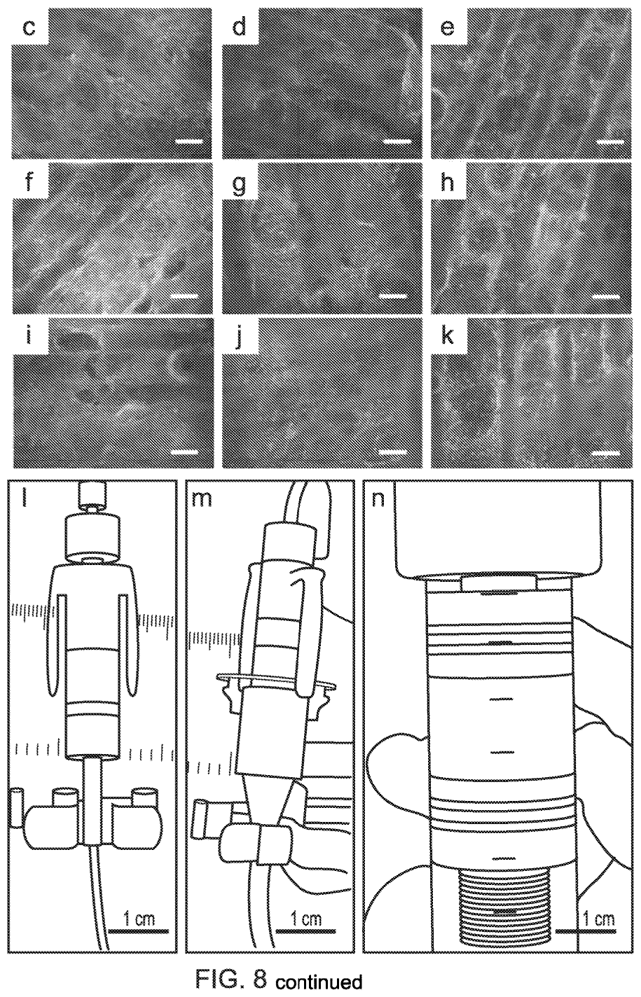

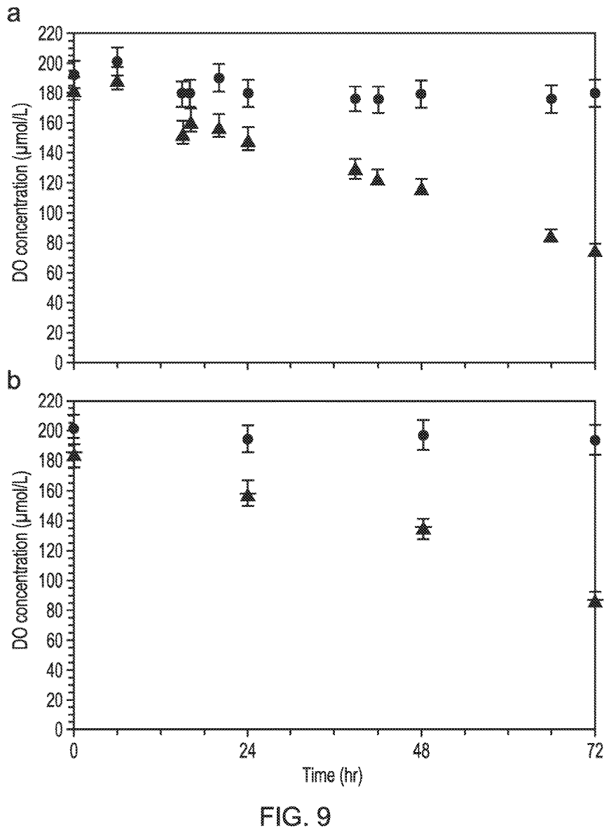

As a further proof of scalability, in a separate set of experiments, dissolved oxygen data from the inlet and outlet of two different scales (8 mm diameter by 10 mm height, and 13 mm diameter and 10 mm height) was collected and compared for a channel flowrate of 0.55±0.05 μL min⁻¹. Comparable trends in consumption of oxygen were observed (FIG. 9). The difference between the dissolved oxygen levels at the inlet and outlet of 8 mm diameter CASP bioreactor increased over the operating time of the reactors; from 34.11±2.63 μmol/L at 24 h, to 62.48±8.63 μmol/L at 48 h and 103.97±11.91 μmol/L at 72 h. For the 13 mm diameter PFB the oxygen drop increased from 36.5±3 μmol/L at 24 h to 61.5±7.63 μmol/L at 48 h and to 106±10.38 μmol/L at 72 h. The oxygen drops at each time point were similar and statistically comparable (p<0.05, one way ANOVA) demonstrating that, as expected, by maintaining the channel flowrate the CASP bioreactor was successfully scaled and the radial oxygen supply could be maintained.

Radisic (2008) [16] and Kaplan (2014) [31] have used the same concept of cylindrical PFBs to grow 3D tissues. Radisic used laser piercing for vascularization while Kaplan used a parallel array of wires in a cylindrical mold to induce parallel channels. Fabricating long and thin channels with lasers is not possible; as a result, the scaffold in Radisic[16] study has perfusion length of only 2 mm. As another disadvantage, since channeling process takes place before seeding, the scaffold would be a channeled scaffold which is impractical to seed in big scales [38], [21]. To solve the seeding problem and simultaneously increase the perfusion length Kaplan [31] used hydrogel cross-linking around an array of wires. Although this solved the two aforementioned bottlenecks of scale-up in PFBs, hydrogels alone may not be suitable for large scale operations for reasons such as; cells settling during the time it takes to cross-link in hydrogel solutions, meaning a uniform cell seeding is unlikely; and furthermore, hydrogels have low mechanical integrity so are at risk of compaction and therefore reduced efficiency of the channels [32], [44], [45]. Moreover, the whole scaffold may shrink up to 5-7 times which completely affect the fluid flow regimes and efficiency in PFBs [44]. The CASP bioreactor described in this paper overcomes these challenges of hydrogels with the polymeric backbone providing the mechanical structure. The hydrogel might still shrink; however this no longer affects the 3D topology of neither the channels, nor the whole scaffold, as they are made of a polymer. Thus, the polymeric backbone, provides a stability of the structure and also is the key factor in making rolling scaffolds possible.

### 2.7. Differentiation

Differentiation was performed on unrolled scaffolds for 7 days and 14 days (for images refer to the supplementary data). After 7 days some cell-confluent areas could be seen; however, the cells were neither fused nor aligned. Almost no myofibers could be witnessed. At 14 days of differentiation, fewer cells and fibrin were detected on the scaffold. It is assumed that fibrin contraction [36], [45] and also dissolution [46] inside the liquid media were the reasons why very few cells could be detected on scaffolds. To back this up, Rowe (2007) [44] states that fibrin shrinks to just one-tenth of its initial volume after 6 days (which is very close to the differentiation time span). While the compaction doesn't change the general shape of the scaffold unlike the similar reports; because, here the hydrogel is backed by the PCL support; yet, in micro-scale we still have it and it impacts the differentiation. Noori (2016) [35] states that cells grown in fibrin tend to take an orientation parallel to the fibril bundles. If fibrin compaction happens in any direction other than the myofibers' striation direction, the induced mechanical force in an undesirable factor that stops the myoblasts to fuse together and elongate. Microfibrils of fibrin tend to contract in random directions which is hypothesized to be an inhibitor of myoblasts' differentiation. Costantini [33] has also mentioned this drawback. As an additional feature, Bensaid (2003) [47] reports that within 15 days of in vitro culture hydrogels of 9 mgr/L fibrin and 50 U/mL thrombin completely dissolve. This could be the reason that no desirable differentiation was detected in current study after 14 days of culture. Many studies propose using of Aprotinin, aminocaproic acid or acetylsalcilic acid as fibrinolysis inhibitors to avoid this phenomena [45, 48]. Although fibrin didn't act as a proper hydrogel in this study, it has shown a great potential to be used for differentiation if it is mechanically stimulated and stretched in one axis. Heher et al [19] report applying uniaxial mechanical forces on a fibrin ring and hence obtain myoblasts fusion and striation. Accordingly, fibrin was still considered as a desirable hydrogel for the CASP system and further studies could apply mechanical stimulation to it so as to improve the differentiation.

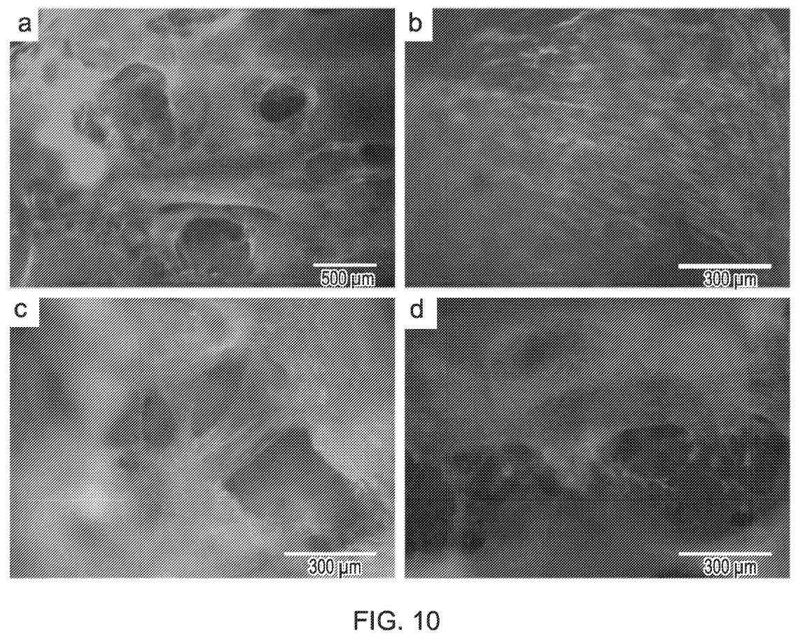

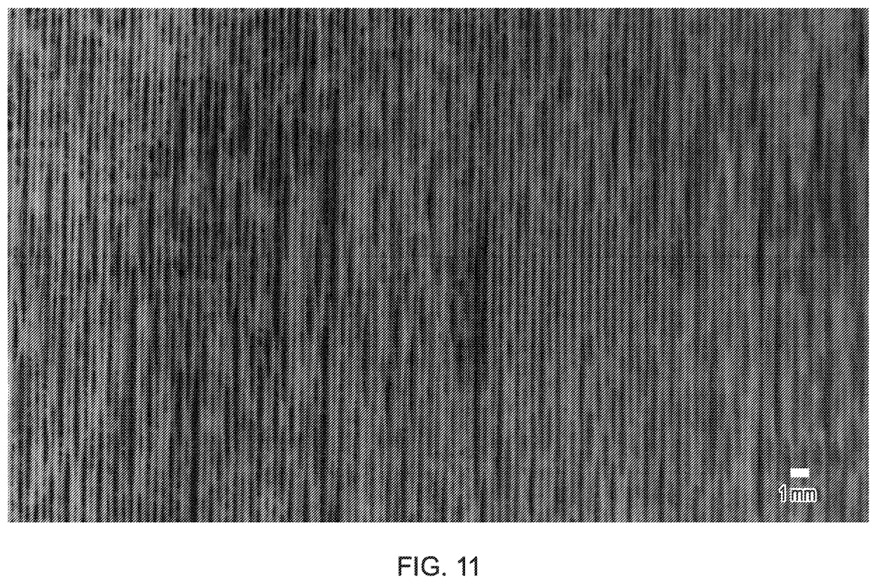

In comparison to differentiation on flat sheets, some striation and fusion of myoblasts was observed in CASP bioreactors (FIG. 10). Formation of some myotubes was also detected in CASP bioreactors after 7 days of differentiation. It can be seen, (FIG. 10. d) that myotubes have detectable striation along the channel. It can be hypothesized that the mechanical force from sheer stresses caused by the fluid helps the striation of the cells. Several researches have shown this positive influence of mechanical stimulations on differentiation [19], [49], [50], [51]. Hydrogels (fibrin and collagen) are prevalently used in the mentioned studies; however, they could curb the negative impacts of hydrogel compaction by hooking two sides of the hydrogel. As a result, microfibrils in parallel with the axis of the mechanical stimulation and thus would not disturb the alignment of cells but help it. Applying a similar methodology to the system described herein is hypothesized to have positive impacts on increasing the differentiation extent.

### 3. Conclusion

This example has outlined the fabrication method for a novel uniformly-pseudovascularized spiral-wound scaffold for cultured meat, CASP. The casting method is rapid and highly controllable, and is capable of producing portion-sized constructs thus achieving a scale that has been previously stated as a bottleneck to cultured meat production. It has been demonstrated that the fabrication method provides a uniform and efficient seeding of large 3D spiral-wound scaffolds which overcomes another previously stated challenge. As another advantage is that the CASP concept can support viable cells in up to 75% of its active volume while this efficiency was reported between 7 to 50% in previously reported channeled PFBs.

The CASP bioreactor system in this study was proven to be able to expand the number of the cells from the initial seeding density (50,000 cell/cm²) up to one million cells/cm²; and hence make the number of the cells 20 times in five days of proliferation. In addition, the removal of cells without reducing viability was demonstrated using animal-free, inexpensive, food grade nattokinase, meaning that the suspended cells (if not used in situ for differentiation and maturation of myotubes) can be used to seed a bioreactor that is 20 times larger by volume. PCL was used to develop the concept of CASP, and its low biodegradability (nearly 2 years) could allow it to be reused. Nonetheless, as initially-stated perspective for growing differentiated whole-cut meat, in the future it is envisage that the CASP construct can be made from a range of materials including edible materials, and will be able to combine cell types, enabling future scaffold design with the potential to fully differentiate the myoblast and incorporated infused vitamins, minerals and flavours into the (edible) scaffold itself.

### Example 2

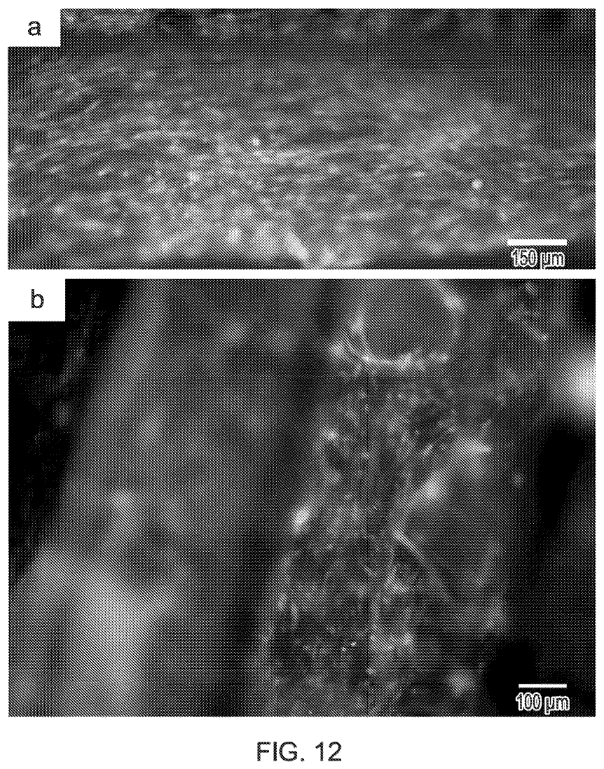

A readily-available non-woven sheet of pp/ps was etched with laser and laminated on another (non-etched) sheet using a layer of PCL. This construct was then sterilized by 70% ethanol aqueous solution and used for seeding cells. Cells were seeded on the mentioned construct supported by a layer of fibrin. Live/dead staining of the cells (after 4 days of proliferation and 6 days of differentiation) shows striation and differentiation (FIG. 12) of cells.

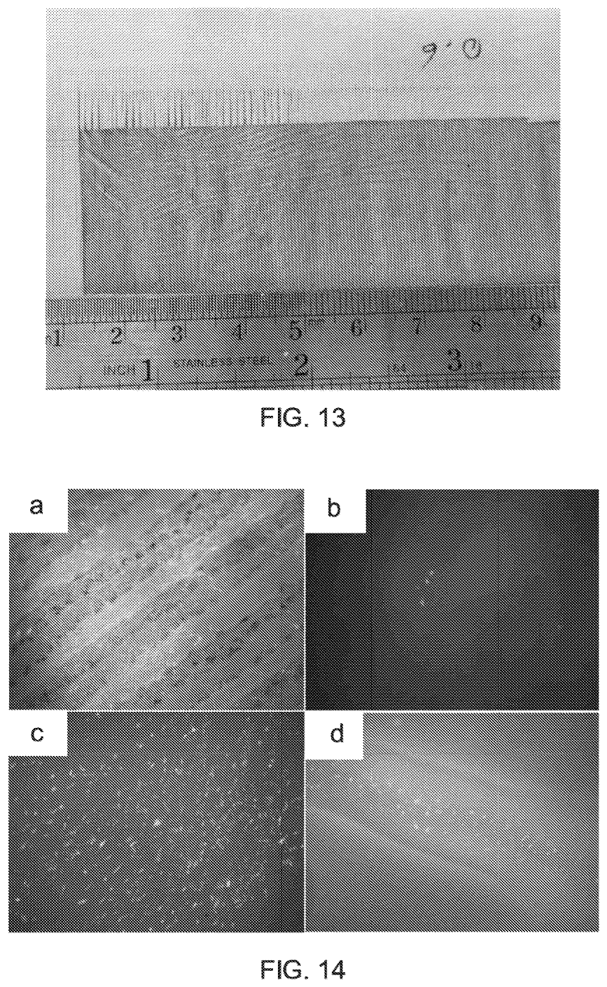

As differentiation on this construct was achieved, the pp/ps polymer was replaced with a digestible polymer. BCS was chosen as it has been shown to be laserable (FIG. 13) and cells could grow and proliferate on it while supported by fibrin or gelatin (FIG. 14 a and d respectively)

Laser-etched BCS Spiral-wound Pseudovascularised construct is a scaffold-bioreactor combinational design allowing rapid and scalable proliferation and differentiation of myoblasts (skeletal muscle cells); and, offering a final product of high protein content and meat-resembling texture. It is a giant step toward “economic cultured meat” as the scaffold is animal-free, edible, highly nutritious (40% protein), cheap and abundant. Furthermore, the scaffold can be made rapidly with highly-controllable standards at industrial scales. Lastly, the pseudovascularised spiral-wound geometry unleashes the opportunity of producing 3D portion-sized meat-alternatives which has been a reported bottleneck in many cultured meat related publications. The combination of all the aforementioned features has not been reported yet elsewhere.

The initial format of the scaffold is a 0.14-mm-thick, flat sheet of soybean curd fabricated by a method known as industrial curding.

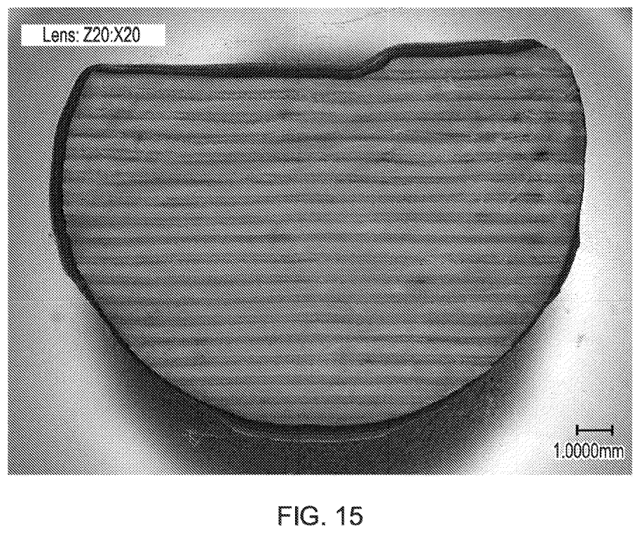

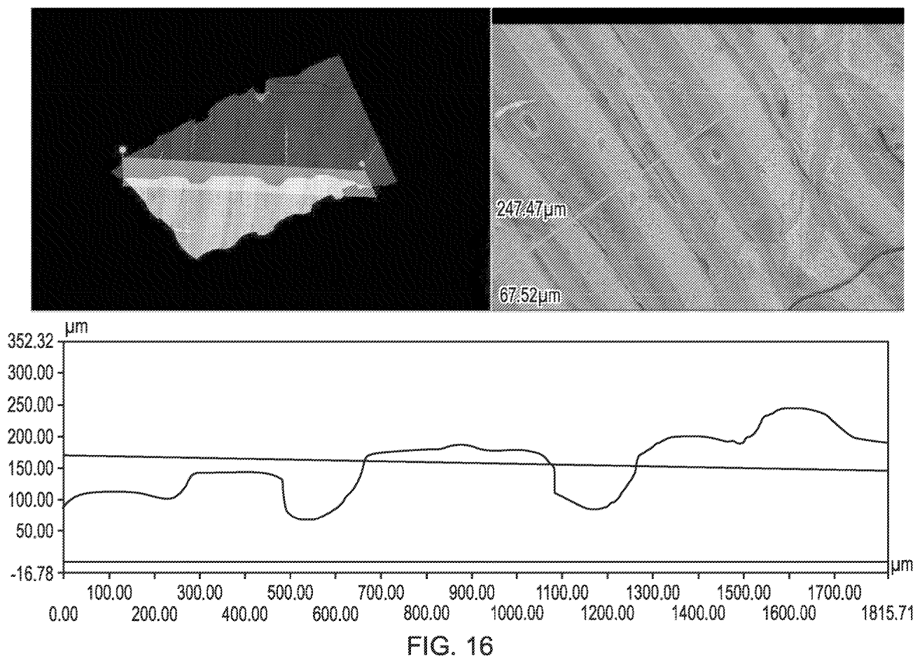

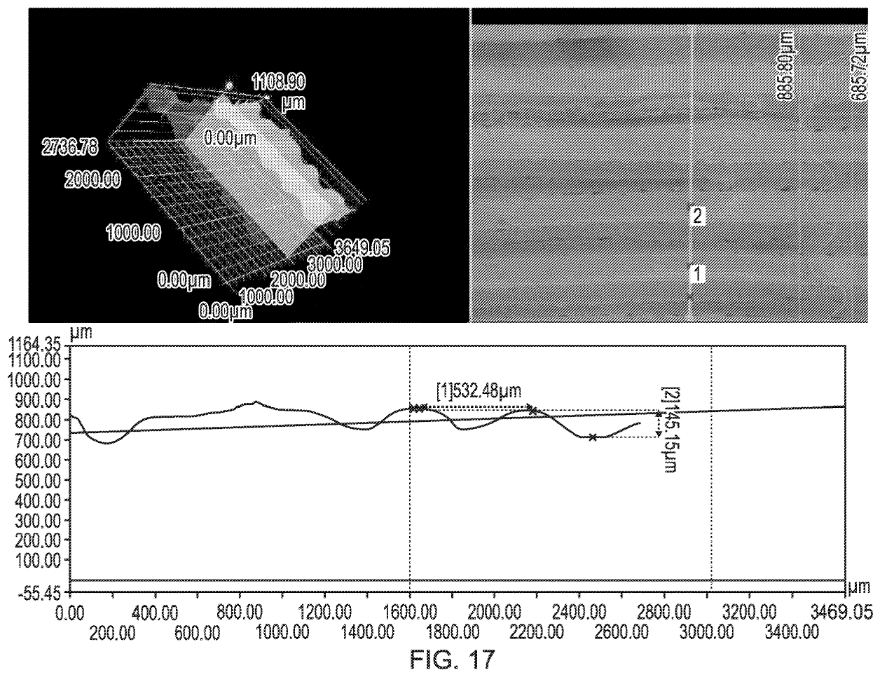

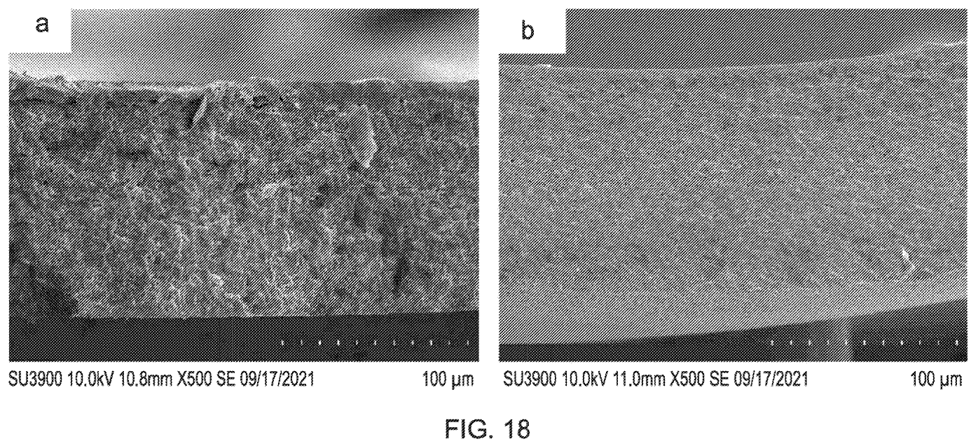

A CO₂ laser machine (Epioglaser®, Fusion M2, 60 W) was used to etch the BCS (0.14 mm thickness) in parallel grooves. OpenScad® coding and COREL design was used to fabricate grooves with 200 μm intervals and 200 μm width (FIG. 13). Etching depth was found to be 100 μm to 140 μm (complete cut trough) (FIGS. 17 and 18). Etched sheets were immersed in 70 mM calcium chloride aqueous solution which is a food-grade firming agent (for 24 h at room temperature) and then cut to thin open-ended stripes. Afterwards, the scaffolds were sterilized in 70% ethanol in water solution (for 24 h at room temperature), rinsed with sterile culture media several times and kept in fresh sterile culture media until seeding. 10 days of exposure to culture media didn't dissolve or disintegrate the scaffold (FIG. 15).

Muscle cells were seeded on the surface of the construct using fibrin hydrogel. C2C12 cell-line was used as a model for proliferation and differentiation of skeletal muscle cells (i.e. the main component of meat). Proliferative C2C12s were suspended in growth media while thrombin enzyme was added to this media. Fibrinogen protein was dissolved in another batch of media (without sera and cells) and was pipetted on top of the scaffold (in sheet format). The mixture of cells and thrombin was added to the scaffolds shortly after fibrin. Aliquots of cells are added to each square centimetre of the scaffold (20,000 cell cm⁻²). The mixture of cells, fibrinogen and thrombin cover the surface of the flat scaffold. After 3 hr, thrombin clots the fibrinogen protein into fibrin (aka. fibrin crosslinking). Fibrin shows appropriate hydrogel properties and hence provides an appropriate environment for cells to grow.

Power and frequency of the laser beam and the speed of the laser head micromotor were figured out in a trial and error. These settings may be different based on the equipment being used (laser machine), BCS thickness and curding process.

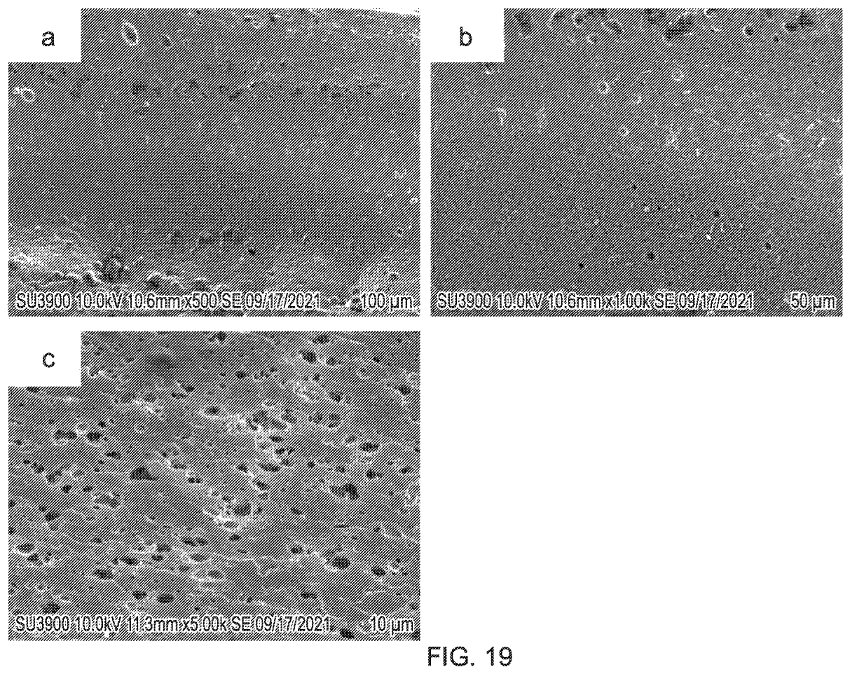

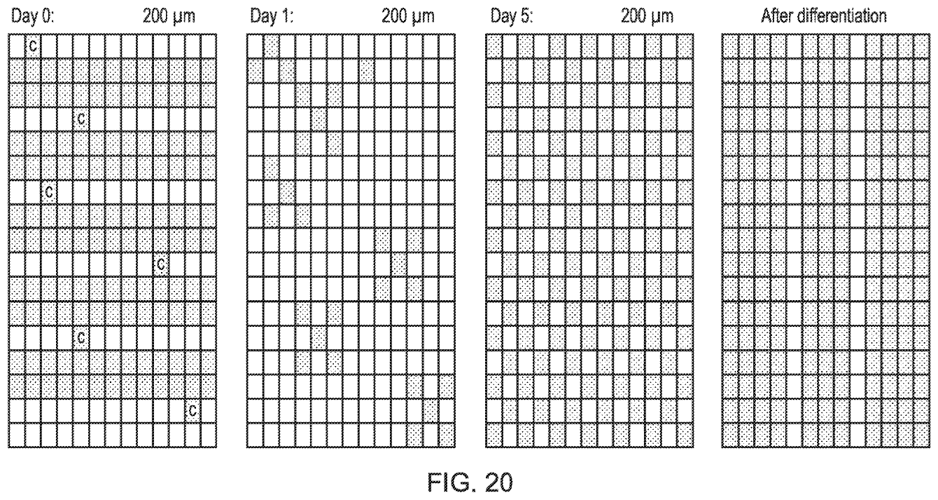

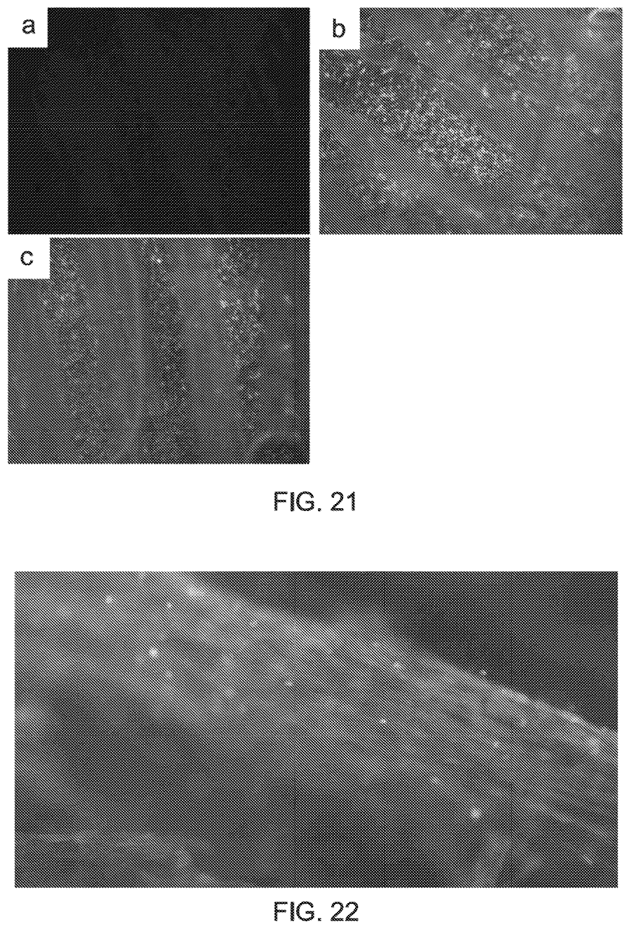

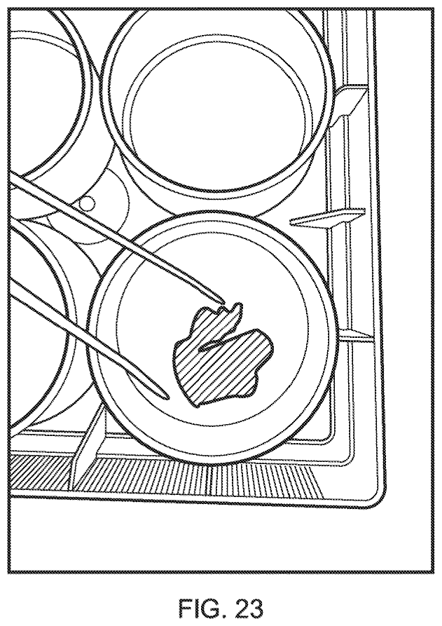

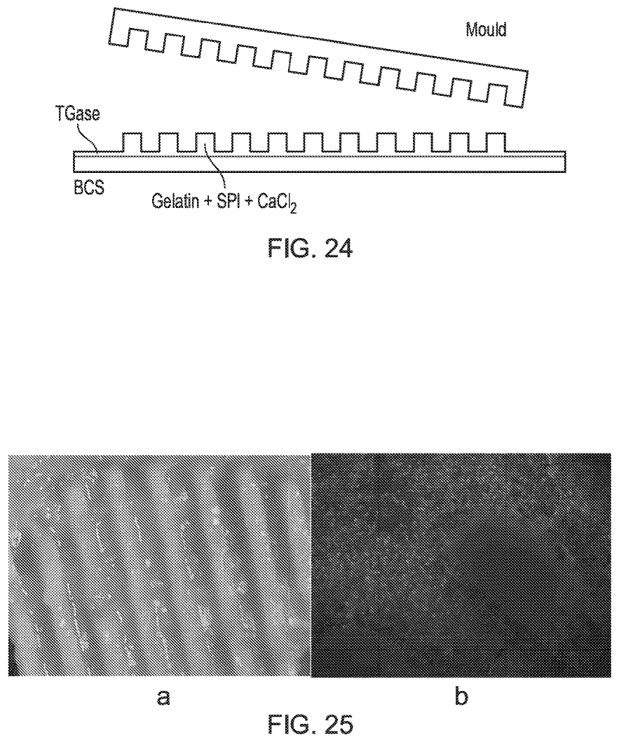

Cells were cultured in proliferation media for 4 days and switched to differentiation media for 6 days afterwards. Staining with Hoechst, propidium iodide (PI) and Fluorescein Diacetate (FDA) and fluorescence microscopy were used to show cells' proliferation (FIG. 21) and differentiation. Fusion of the myoblasts together and their parallel alignments are demonstrative of differentiation (FIG. 22).

Additional to the first generation of CASP, here, culturing in differentiation media led to proper aligned striated and fused format. The parallel shape of the grooves was hypothesized to be the differentiation stimulator. Protein assays performed in, showed a significant increase in cell's protein content (at least 2.33 fold) after differentiation (compared to their proliferative state) leading to a higher ratio of animal to plant protein in the final product. Animal-source proteins have a positive impact on consumers' skeletal muscle mass due to their more favorable effect on protein synthesis, attributed partly to their higher digestibility and content of leucine, lysine, methionine as compared to plant proteins. Also, vitamin B12 and heme iron are only provided in animal meats and not the plant alternatives.

### Example 3—Coated BCS Construct

A digestible polymer sheet, in this example BCS, was coated with transglutaminase (TGase). The TGase was applied dry. A hot solution of gelatin, soy protein isolate and calcium chloride was poured on top and a mould was pressed onto the hot solution to mould it into formations defining channels between adjacent formations.

After the mould was pressed onto the hot solution the assembly was gelled for 1 hour at 4 degrees Celsius. After gelling the mould was removed and the cooled gelled solution maintained its form. The construct was then frozen at −20 degrees Celsius and then freeze dried.

A cross-linking solution of 1-ethyl-3-(3-dimethyl aminopropyl) carbodiimide (EDC) and N-hydroxy succinimide (NHS) was prepared by mixing 25 mM of EDC and 10 mM of NHS into 100 mL of a mixture of ethanol/distilled water (9:1 v/v). The cross-linking solution was applied to the construct to crosslink/couple/cure/harden amines to improve the structure and to increase the hardness of the surface. This also acts as a sterilization step. Alternatively, TGase could have been applied for the same reasons.

The construct was then rinsed with a mix of sterile culture media and phosphate-buffered saline (PBS) to remove soluble or loose particulates. The construct was also rinsed with ethanol. The resultant construct is shown in FIG. 25(a).

A concentrated cell pellet was spread onto the surface of the construct and left for 3 hours to allow for cell attachment. The construct was then slowly immersed in cell culture media and left for 4 days. FIG. 25(b) shows that cells attached to the construct in the channels and proliferated over the 4 days.

### Example 4—Mechanical Properties Testing

Young's Modulus (E) is a gauge for materials' stiffness, with above 1050 GPa for diamond as one of the hardest materials on earth to 10 MPa for rubber (very elastic and soft). Lower Young's modulus represents softer materials which can be bent relatively easier than hard materials such as steel (E=200 GPa). Young's modulus is different from “hardness” (relative resistance of the material's surface to penetration by a harder body) and “toughness” (amount of energy that a material can absorb before fracture); and a better representative of elasticity. The strain-stress curve drawn to calculate E can also be used to find the reversible elasticity domain of a material. Reversibility of elastic deformation means that the material returns to its original shape after the load is removed (i.e. is a rollable and/or foldable material). It is shown that materials suitable for the scaffolds having relatively low young's modulus are bendable and rollable without any fracture or tears.

### Methods

An Instron 3369 (USA) machine was used as an instrument for this purpose. It was equipped with BlueHill universal software. For the sake of small, subtle samples, small (1 cm wide) 50 N pneumatic grips were used to hold the samples from two ends (and stretch them). The stretching rate was set on 0.5 mm per min, while a 50 N load cell was used to measure the applied force every tenth of a second.

The samples were in sheet format and were cut into rectangles with width of 0.6 to 1 cm and length of 1 to 3 cm. The width and length of each and every sample was measured separately before the experiment (using calipers with accuracy of 0.01 mm). The thickness of samples was also measured by using the same calipers and also image processing of their SEM characterization. Although the length and width of each sample (of the same material) were different due to manual cutting; the thickness was constant within the samples of a same material.

As the software was giving a force vs displacement (rather than the more conventional graph of strain-stress which can give the Young's modulus directly), the E was calculated as demonstrated below:

$$E = \frac{σ ( ε )}{ε} = \frac{F / A}{Δ L / {L}_{0}} \to F = \frac{E A Δ L}{{L}_{0}} \to slope of the line is \frac{E A}{{L}_{0}}$$

Slope of the line in the given graphs was timed by L₀ and divided by the surface of sample (A=width*thickness) to obtain E. The width, length and thickness of each sample were measured as stated above.

### Results

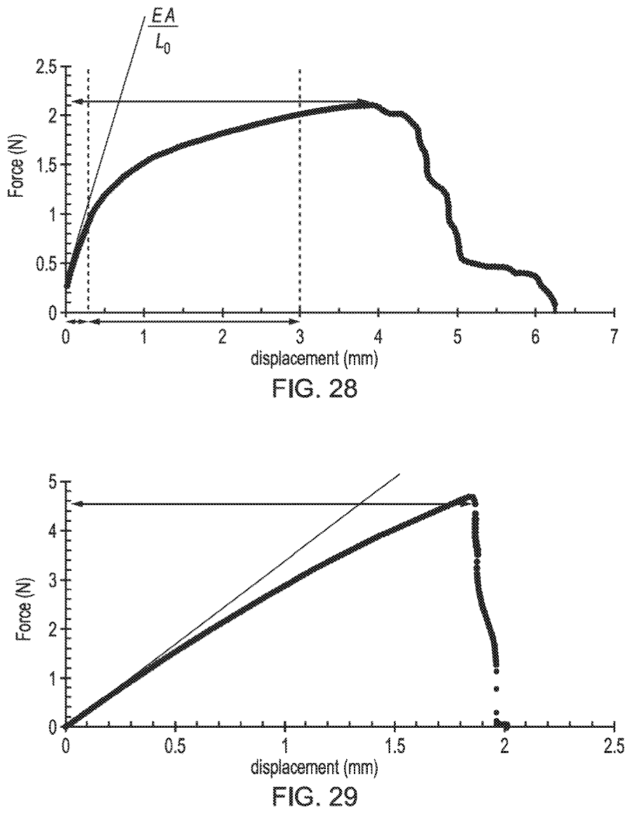

With reference to the force-displacement graph for porous sheets of PCL shown in FIG. 28. The elastic domain (A), plastic range (B), and how to obtain E from this graph are demonstrated. It also presents elongation at break; C (which should be divided by the original length and timed by 100 to give the percentage).

The thicknesses of the samples were assessed as follows; 130±10 μm, 140±10 μm, 90±10 μm and 500±30 μm for Novatexx 2471 nonwoven polypropylene backing layer, BCS (wet or dry), non-porous 8% PCL sheets and Porous PCL sheets (as described in Examples 1 and 2) respectively. The other dimensions are demonstrated in Table 1:

TABLE 1
Dimensions of samples for mechanical testing

| Sample | Width (mm) | Length (mm) (L₀) |
|---|---|---|
| Novatexx (1) | 6.10 | 28 |
| Novatexx (1) | 8.30 | 20 |
| PCL 8% non-porous (1) | 8.00 | 20 |
| PCL 8% non-porous (2) | 8.40 | 18 |
| Porous PCL (1) | 8.20 | 12 |
| Porous PCL (1) | 9.10 | 14 |
| BCS (dry-1) | 9.20 | 20 |
| BCS (dry-2) | 10.50 | 28 |
| BCS (wet-1) | 10.00 | 30 |
| BCS (wet-2) | 10.00 | 25 |

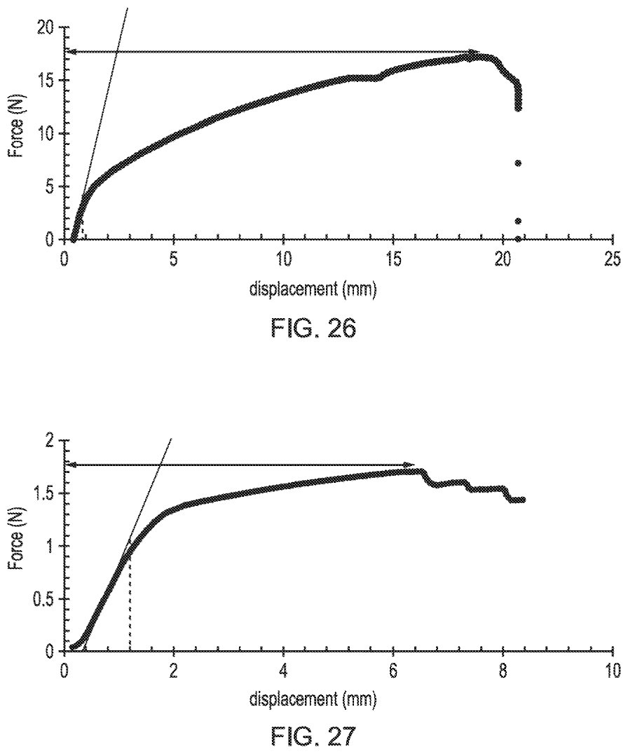

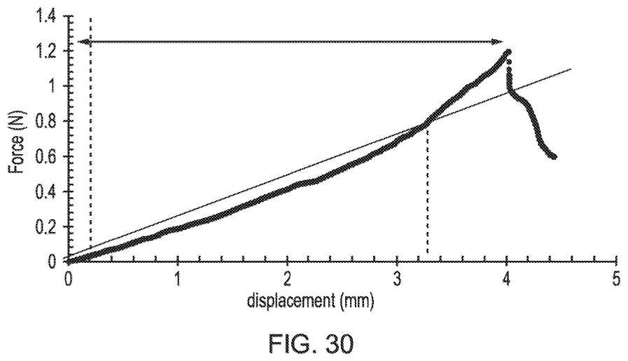

The mechanical characterization for each material are shown in Table 2 (calculated from the force-displacement graphs shown in FIGS. 26 to 30):

TABLE 2
mechanical properties of materials

| | E (MPa) (Young's | Elongation | Elastic area |
|---|---|---|---|
| Sample | modulus) | at Break | (region) |
| Novatexx (1) | 340-358 | 64-71% | 1.25% |
| PCL 8% non- | 280 | 44% | 5% |
| porous (2) | | | |
| Porous PCL (1) | 106-117 | 15-25% (batch to | 1.4-2% |
| | | batch variation) | |
| BCS (dry-1) | 718-753 | 6% | N/A |
| BCS (wet-1) | 38.5 | 10-16% to 25% | 7-10% |
| | | (batch to batch | |
| | | variation) | |

### REFERENCES

1. Datar, I. and M. Betti, Possibilities for an in vitro meat production system. Innovative Food Science & Emerging Technologies, 2010. 11(1): p. 13-22.
2. Post, M. J. and J. F. Hocquette, Chapter 16 —New Sources of Animal Proteins: Cultured Meat, in New Aspects of Meat Quality, P. P. Purslow, Editor. 2017, Woodhead Publishing. p. 425-441.
3. Stephens, N. and M. Ellis, Cellular agriculture in the UK: a review [version 1; peer review: 2 approved, 2 approved with reservations]. Wellcome Open Research, 2020. 5(12).
4. Allan, S. J., P. A. De Bank, and M. J. Ellis, Bioprocess Design Considerations for Cultured Meat Production With a Focus on the Expansion Bioreactor. Frontiers in Sustainable Food Systems, 2019. 3(44).
5. Humbird, D., Scale-up Economics for Cultured Meat: Techno-economic Analysis and Due Diligence. engrXiv, Web., 29 Dec. 2020.
6. Post, M. J., et al., Scientific, sustainability and regulatory challenges of cultured meat. Nature Food, 2020. 1(7): p. 403-415.
7. Smith, J. H., Relation of Body Size to Muscle Cell Size and Number in the Chicken1,2. Poultry Science, 1963. 42(2): p. 283-290.
8. Koumans, J. T. M., et al., Numbers of muscle nuclei and myosatellite cell nuclei in red and white axial muscle during growth of the carp (Cyprinus carpio). Journal of Fish Biology, 1994. 44(3): p. 391-408.
9. Bruusgaard, J. C., et al., Number and spatial distribution of nuclei in the muscle fibres of normal mice studied in vivo. The Journal of physiology, 2003. 551(Pt 2): p. 467-478.
10. Agarwal, M., et al., Myosin heavy chain-embryonic regulates skeletal muscle differentiation during mammalian development. Development, 2020. 147(7).
11. Hanga, M. P., et al., Bioprocess development for scalable production of cultivated meat. Biotechnology and Bioengineering, 2020. 117(10): p. 3029-3039.
12. Verbruggen, S., et al., Bovine myoblast cell production in a microcarriers-based system. Cytotechnology, 2018. 70(2): p. 503-512.
13. Wassenaar, J. W., G. R. Boss, and K. L. Christman, Decellularized skeletal muscle as an in vitro model for studying drug-extracellular matrix interactions. Biomaterials, 2015. 64: p. 108-14.
14. Al-Shammari, A. A., E. A. Gaffney, and S. Egginton, Re-evaluating the use of Voronoi Tessellations in the assessment of oxygen supply from capillaries in muscle. Bull Math Biol, 2012. 74(9): p. 2204-31.
15. Kang, Y. and J. Chang, Channels in a porous scaffold: a new player for vascularization. Regen Med, 2018. 13(6): p. 705-715.
16. Radisic, M., et al., Cardiac tissue engineering using perfusion bioreactor systems. Nat Protoc, 2008. 3(4): p. 719-38.
17. Mofrad, A. Z., S. Mashayekhan, and D. Bastani, Simulation of the effects of oxygen carriers and scaffold geometry on oxygen distribution and cell growth in a channeled scaffold for engineering myocardium. Math Biosci, 2017. 294: p. 160-171.
18. Allenby, M. C., et al., Dynamic human erythropoiesis in a three-dimensional perfusion bone marrow biomimicry. Biomaterials, 2019. 188: p. 24-37.
19. Heher, P., et al., A novel bioreactor for the generation of highly aligned 3D skeletal muscle-like constructs through orientation of fibrin via application of static strain. Acta Biomater, 2015. 24: p. 251-65.
20. Guarino, V., F. Causa, and L. Ambrosio, Porosity and mechanical properties relationship in PCL porous scaffolds. J Appl Biomater Biomech, 2007. 5(3): p. 149-57.
21. Marin, A. C., et al., micro-Particle tracking velocimetry and computational fluid dynamics study of cell seeding within a 3D porous scaffold. J Mech Behav Biomed Mater, 2017. 75: p. 463-469.
22. Tocchio, A., et al., Versatile fabrication of vascularizable scaffolds for large tissue engineering in bioreactor. Biomaterials, 2015. 45: p. 124-31.
23. Iwasaki, K., et al., Bioengineered three-layered robust and elastic artery using hemodynamically-equivalent pulsatile bioreactor. Circulation, 2008. 118(14 Suppl): p. S52-7.
24. Li, J.-F., et al., Effect of TiO2 nanoparticles on the surface morphology and performance of microporous PES membrane. Applied Surface Science, 2009. 255(9): p. 4725-4732.
25. Onder, O. C., E. Yilgor, and I. Yilgor, Preparation of monolithic polycaprolactone foams with controlled morphology. Polymer, 2018. 136: p. 166-178.
26. Frost, H. K., et al., Electrospun nerve guide conduits have the potential to bridge peripheral nerve injuries in vivo. Scientific Reports, 2018. 8(1): p. 16716.
27. Jeffries, E. M. and Y. Wang, Incorporation of parallel electrospun fibers for improved topographical guidance in 3D nerve guides. Biofabrication, 2013. 5(3): p. 035015.
28. Sasmazel, H. T., et al., Comparison of cellular proliferation on dense and porous PCL scaffolds. Biomed Mater Eng, 2008. 18(3): p. 119-28.
29. Meneghello, G., et al., Fabrication and characterization of poly(lactic-co-glycolic acid)/polyvinyl alcohol blended hollow fibre membranes for tissue engineering applications. Journal of Membrane Science, 2009. 344(1): p. 55-61.
30. Tang, Z. G., et al., Surface properties and biocompatibility of solvent-cast poly[ε-caprolactone] films. Biomaterials, 2004. 25(19): p. 4741-4748.
31. Rnjak-Kovacina, J., et al., Arrayed Hollow Channels in Silk-Based Scaffolds Provide Functional Outcomes for Engineering Critically Sized Tissue Constructs. Vol. 24. 2014.
32. Pourchet, L., et al., Large 3D bioprinted tissue: Heterogeneous perfusion and vascularization. Bioprinting, 2019. 13: p. e00039.
33. Costantini, M., et al., Microfluidic-enhanced 3D bioprinting of aligned myoblast-laden hydrogels leads to functionally organized myofibers in vitro and in vivo. Biomaterials, 2017. 131: p. 98-110.
34. Choi, Y.-J., et al., A 3D cell printed muscle construct with tissue-derived bioink for the treatment of volumetric muscle loss. Biomaterials, 2019. 206: p. 160-169.
35. Noori, A., et al., A review of fibrin and fibrin composites for bone tissue engineering. Int J Nanomedicine, 2017. 12: p. 4937-4961.
36. Cummings, C. L., et al., Properties of engineered vascular constructs made from collagen, fibrin, and collagen-fibrin mixtures. Biomaterials, 2004. 25(17): p. 3699-706.
37. Villalona, G. A., et al., Cell-seeding techniques in vascular tissue engineering. Tissue Eng Part B Rev, 2010. 16(3): p. 341-50.
38. Maidhof, R., et al., Perfusion seeding of channeled elastomeric scaffolds with myocytes and endothelial cells for cardiac tissue engineering. Biotechnol Prog, 2010. 26(2): p. 565-72.
39. Pankajakshan, D., et al., Development of a fibrin composite-coated poly(epsilon-caprolactone) scaffold for potential vascular tissue engineering applications. J Biomed Mater Res B Appl Biomater, 2008. 87(2): p. 570-9.
40. Kang, S. W., et al., Surface modification with fibrin/hyaluronic acid hydrogel on solid-freeform-based scaffolds followed by BMP-2 loading to enhance bone regeneration. Bone, 2011. 48(2): p. 298-306.
41. Carpenter, C., B. Rodriguez, and N. Cockett, Growth and differentiation of cultured satellite cells from callipyge and normal lambs. Canadian Journal of Animal Science, 2000. 80(2): p. 297-302.
42. Cheng, C. S., et al., Conditions that promote primary human skeletal myoblast culture and muscle differentiation in vitro. Am J Physiol Cell Physiol, 2014. 306(4): p. C385-95.
43. Beşkardş, I. G., et al., A systematic study for optimal cell seeding and culture conditions in a perfusion mode bone-tissue bioreactor. Biochemical Engineering Journal, 2018. 132: p. 100-111.
44. Rowe, S. L., S. Lee, and J. P. Stegemann, Influence of thrombin concentration on the mechanical and morphological properties of cell-seeded fibrin hydrogels. Acta Biomater, 2007. 3(1): p. 59-67.
45. Hinds, S., et al., The role of extracellular matrix composition in structure and function of bioengineered skeletal muscle. Biomaterials, 2011. 32(14): p. 3575-83.
46. Marcinczyk, M., et al., Laminin-111 enriched fibrin hydrogels for skeletal muscle regeneration. Biomaterials, 2017. 141: p. 233-242.
47. Bensaid, W., et al., A biodegradable fibrin scaffold for mesenchymal stem cell transplantation. Biomaterials, 2003. 24(14): p. 2497-2502.
48. Chiu, C. L., et al., Permeability of three-dimensional fibrin constructs corresponds to fibrinogen and thrombin concentrations. Biores Open Access, 2012. 1(1): p. 34-40.
49. Okano, T., et al., Tissue engineering of skeletal muscle. Highly dense, highly oriented hybrid muscular tissues biomimicking native tissues. Asaio j, 1997. 43(5): p. M749-53.
50. Powell, C. A., et al., Mechanical stimulation improves tissue-engineered human skeletal muscle. Am J Physiol Cell Physiol, 2002. 283(5): p. C1557-65.
51. Beldjilali-Labro, M., et al., Biomaterials in Tendon and Skeletal Muscle Tissue Engineering: Current Trends and Challenges. Materials (Basel), 2018. 11(7).

1. A construct for perfusion bioreactor cell culture comprising:
at least one flexible polymer sheet for rolling or folding in use;
a plurality of channels for transport of cell culture medium to cells and/or hosting cells in use.

2. The construct of claim 1, wherein the construct is suitable for cell proliferation, cell differentiation and/or harvesting of cells.

3. The construct of claim 1 or 2, wherein the at least one flexible polymer sheet is non-porous or comprises an average pore diameter from 0 to 49 μm and the channels are open channels,
optionally wherein each of the plurality of open channels comprise an average width from 20 μm to 1000 μm,
further optionally wherein each of the plurality of open channels comprise an average depth from 50 μm to 500 μm;
further optionally wherein the at least one flexible polymer sheet comprises an average thickness from 30 μm to 1000 μm.

4. The construct of claim 3, wherein the construct comprises a laminate structure, wherein the at least one flexible polymer sheet comprises a first flexible polymer sheet and a second flexible polymer sheet adhered to each other, and wherein the first or second flexible polymer sheet comprises the plurality of open channels.

5. The construct of claim 1 or 2, wherein the at least one flexible polymer sheet comprises a porous polymer comprising pores of an average diameter of 100 μm to 600 μm and wherein the channels are closed channels;
optionally wherein each of the plurality of closed channels have an average width from 5 μm to 1000 μm,
further optionally wherein the porous polymer has an average thickness from 100 μm to 1000 μm

6. The construct of claim 5, wherein:
a) each of the plurality of closed channels are hollow; or
b) each of the plurality of closed channels comprise a channel forming member disposed therein.

7. The construct of any preceding claim, wherein each of the plurality of channels are spaced apart by an average distance from 100 μm to 1500 μm;
optionally wherein the channels extend longwise from one surface of the flexible polymer sheet to an opposing surface of the flexible polymer sheet;
further optionally wherein the channels are substantially parallelly aligned, optionally wherein the each of the channels comprises a uniform cross-section.

8. The construct of any preceding claims, wherein the at least one flexible polymer sheet comprises a biodegradable polymer;
optionally wherein the at least one flexible polymer sheet comprises an edible polymer;
further optionally wherein the at least one flexible polymer sheet comprises a digestible polymer.

9. The construct of any one of claims 1 to 4 and 7 to 8, wherein the at least one flexible polymer sheet comprises bean curd sheet (BCS).

10. The construct of claim 9, wherein the plurality of channels are moulded on the BCS flexible polymer sheet.

11. The construct of any of claims 1 to 10, wherein the at least one flexible polymer sheet comprises a polymer selected from at least one of polycaprolactone, polypropylene and/or polystyrene.

12. A method of forming a construct for perfusion bioreactor cell culture, the method comprising the steps of:
a) dissolving a polymer in a solvent to form a mixture;
b) mixing a porogen having a diameter of at most 125 μm into the mixture;
c) applying the mixture onto a planar structure;
d) immersing the planar structure comprising the applied mixture in an anti-solvent; and drying the planar structure comprising the applied mixture to form a porous flexible polymer sheet for rolling or folding in use;
wherein the planar structure comprises a plurality of channel forming members and wherein the mixture is applied so that the mixture encases the channel forming members; and
e) removing the construct comprising the channel forming members therein from the planar structure.

13. The method of claim 12, wherein the channel forming members of the planar structure comprise a plurality of wires, optionally wherein the wires comprise an average diameter from 5 μm to 1000 μm, further optionally wherein the wires comprise an average diameter of between 270 μm to 340 μm.

14. The method of claim 12 or 13, further comprising a step of removing the channel forming members from the construct to provide closed channels.

15. The method of any one of claims 12 to 14, wherein each of the plurality of channel forming members are spaced apart by an average distance of between 100 μm and 1500 μm.

16. The method of any one of claims 12 to 15, wherein the solvent comprises acetone.

17. The method of any one of claims 12 to 16, wherein the porogen comprises salt particles, optionally the salt particles comprise sodium chloride (NaCl).

18. The method of any one of claims 12 to 17, wherein the porous flexible polymer sheet comprises pores of an average pore diameter of 100 μm to 600 μm;
optionally wherein the porous flexible polymer sheet has an average thickness of between 300 μm and 1000 μm.

19. A method of forming a construct for perfusion bioreactor cell culture, the method comprising the steps of:
a) providing at least one flexible polymer sheet for rolling or folding in use;
b) etching a plurality of channels onto at least one surface of the at least one flexible polymer sheet, wherein each channel extends from one surface of the at least one flexible polymer sheet to an opposing surface of the flexible polymer sheet and wherein each of the channels are open channels.

20. The method of claim 19, wherein the channels extend through the at least one surface of the at least one flexible polymer sheet and the method further comprises:
c) adhering the at least one flexible polymer sheet comprising the plurality of channels extending therethrough to a second flexible polymer sheet to form a laminate structure.

21. The method of claim 19 or 20, wherein the plurality of channels are etched onto the at least one surface and extend between two opposing surfaces of the at least one flexible polymer sheet and the method further comprises cutting the at least one flexible polymer sheet or the laminate structure of claim 20 so each channel extends from one surface of the at least one flexible polymer sheet to an opposing surface of the flexible polymer sheet;
optionally wherein the at least one flexible polymer sheet and second flexible polymer sheet are adhered by a flexible polymer.

22. The method of any one of claims 19 to 21, wherein each of the plurality of channels are spaced apart by an average distance of between 100 μm and 1500 μm.

23. The method of any one of claims 19 to 22, wherein the at least one flexible polymer sheet and/or the second flexible polymer sheet comprises an average thickness from 30 μm to 1000 μm.

24. The method of any one of claims 19 to 23, wherein the at least one flexible polymer sheet comprises an average pore diameter from 0 to 49 μm;
optionally wherein each of the plurality of channels comprise an average width from 20 μm to 1000 μm;
further optionally wherein each of the plurality channels comprise an average depth from 50 μm to 500 μm;
further optionally wherein the channel forming members or channels are substantially parallelly aligned.

25. The method of any one of claims 12 to 24, wherein the porous flexible polymer sheet or the at least one flexible polymer sheet and/or second flexible polymer sheet comprise a biodegradable polymer;
optionally wherein the porous flexible polymer sheet or the at least one flexible polymer sheet and/or second flexible polymer sheet comprise an edible polymer;
further optionally wherein the porous flexible polymer sheet or the at least one flexible polymer sheet and/or second flexible polymer sheet comprise a digestible polymer.

26. The construct of any of claims 12 to 24 wherein the porous flexible polymer sheet or the at least one flexible polymer sheet and/or second flexible polymer sheet comprise a polymer selected from at least one of polycaprolactone, polypropylene and/or polystyrene.

27. The method of any one of claims 19 to 24, wherein the at least one flexible polymer sheet comprises bean curd sheet (BCS).

28. The method of claim 27, wherein the method further comprises:
immersing the at least one flexible polymer sheet or the laminate structure of claim 21, in a firming agent;
optionally wherein the firming agent comprises a food grade firming agent;
further optionally wherein the firming agent comprises calcium chloride aqueous solution.

29. A construct obtainable by the method according to any one of claims 12 to 28.

30. A system for perfusion bioreactor cell culture comprising:
a) a chamber having an internal space comprising an internal width;
b) a construct according to any one of claims 1 to 6A, 7 to 11 or obtainable by the method according to any one of claims 12 to 28, comprising cells seeded thereon,
wherein the construct is disposed within the internal space of the chamber, and wherein the construct is configured as a filled 3-dimensional structure; and
c) a system for perfusing culture media through the chamber and/or the construct.

31. The system of claim 30, wherein the construct has a width substantially the same as the internal width of the chamber.

32. The system of any one of claim 30 or 31, wherein the system for perfusing media comprises a pumping system.

33. A method of culturing cells comprising the steps of:
a) applying a cell support agent to a construct according to any one of claims 1 to 11 or claim 29;
b) seeding cells on the construct;
c) subjecting the construct to conditions suitable to crosslink the cell support agent;
i) when the construct comprises channel forming members, removing the channel forming members;
d) manipulating the construct to form a filled 3-dimensional structure;
e) disposing the manipulated construct into a chamber of a system for perfusion bioreactor cell culture; and
f) perfusing culture media through the system and maintaining the manipulated construct under conditions suitable for culturing the cells and culturing the cells thereon or therein.

34. The method of claim 33, wherein the system for perfusion bioreactor is a chamber having an internal space comprising an internal width and wherein the construct has a width substantially the same as the internal width of the chamber.

35. The method of claim 33 or 34, further comprising after step f):
g) removing the construct from the chamber and applying a cell support agent-specific enzyme to the construct for releasing the cells from the construct; and
h) recovering the cells.

36. The method of claim 35, wherein the recovered cells are formed into a comestible product;
optionally wherein the comestible product is a meat analogue.

37. The method of any one of claims 33 to 36, wherein the cell support agent is fibrin, and optionally wherein the cell support agent-specific enzyme comprises nattokinase.

38. The system of any one of claims 30 to 32 or the method of any one of claims 33 to 37, wherein the 3-dimensional structure is a cylindrical structure.

39. The method of any one of claims 33 to 38, wherein culturing the cells comprises cell proliferation and/or cell differentiation, optionally wherein perfusing culture media comprises perfusing a first culture media for cell proliferation and/or perfusing a second culture media for cell differentiation.

40. The method of any one of claims 33 to 34 or 37 to 39, wherein the construct comprises at least one flexible polymer sheet comprising an edible polymer and wherein the construct is removed from the chamber and the construct and the cells cultured thereon are formed into a comestible product.

41. The method of any one of claims 33 to 40, wherein manipulating the construct comprises rolling the construct.

42. The system of any one of claims 30 to 32 or the method according to any one of claims 33 to 41, wherein the construct is according to any one of claims 1 to 4 and 7 to 11 and wherein the cells are seeded within the plurality of channels; or
wherein the construct is according to any one of claims 5 to 11 and wherein the cells are seeded within the pores of the porous polymer.

43. The system of any one of claims 30 to 32 or the method according to any one of claims 33 to 42, wherein the cells comprise muscle cells.

44. A comestible product obtainable by the method according to claim 40 and any claims dependent thereon.
